(19) United States
(12) Patent Application Publication
(10) Pub. No.: US 2026/0275312 A1
(43) Pub. Date: Sep. 17, 2026

(54) CRISPR NUCLEASE POLYPEPTIDES AND GENE EDITING SYSTEMS COMPRISING SUCH

(71) Applicant: ARBOR BIOTECHNOLOGIES, INC., Cambridge, MA (US)

(72) Inventors: Lauren E. Alfonse, Cambridge, MA (US); Zachary J. Maben, Cambridge, MA (US); Chad D. Torgerson, Cambridge, MA (US); Kyle E. Watters, Cambridge, MA (US)

(21) Appl. No.: 19/469,508

(22) PCT Filed: Mar. 29, 2024
(86) PCT No.: PCT/US2024/022146

§ 371 Date: Sep. 26, 2025

Related U.S. Application Data
(60) Provisional application No. 63/493,454, filed on Mar. 31, 2023.
(60) Provisional application No. 63/494,682, filed on Apr. 6, 2023.
(60) Provisional application No. 63/494,679, filed on Apr. 6, 2023.
(60) Provisional application No. 63/581,351, filed on Sep. 8, 2023.
(60) Provisional application No. 63/562,135, filed on Mar. 6, 2024.

(51) Int. Cl. C12N 9/22; C12N 15/11; C12N 15/88

(52) U.S. Cl. CPC C12N 9/226; C12N 15/111; C12N 15/88; C07K 2319/09; C12N 2310/20

(57) ABSTRACT

CRISPR nuclease polypeptides derived from a reference CRISPR nuclease set forth as SEQ ID NO: 1, SEQ ID NO: 49, or SEQ ID NO: 64, which share an amino acid sequence at least 90% identical to the reference CRISPR nuclease, and optionally comprises one or more mutations relative to the reference CRISPR nuclease. Also provided herein are gene editing systems comprising such CRISPR nuclease polypeptides and gene editing methods using such.

### CROSS REFERENCE TO RELATED APPLICATIONS

This application claims the benefit of the filing dates of U.S. Provisional Application No. 63/494,682, filed Apr. 6, 2023, U.S. Provisional Application No. 63/581,351, filed Sep. 8, 2023, U.S. Provisional Application No. 63/493,454, filed Mar. 31, 2023, U.S. Provisional Application No. 63/562,135, filed Mar. 6, 2024, and U.S. Provisional Application No. 63/494,679, filed Apr. 6, 2023. Each of the priority applications is incorporated by reference herein in their entities.

### SEQUENCE LISTING

The instant application contains a Sequence Listing which has been filed electronically in XML format and is hereby incorporated by reference in its entirety. Said XML copy, created on Mar. 29, 2024, is named 063586-515001WO_SeqList_ST26.xml and is 167,550 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

A CRISPR-Cas system typically comprises a CRISPR nuclease and one or more RNA components that direct the CRISPR nuclease to a target genomic site for gene editing. It is of interest to develop efficient CRISPR nucleases to improve gene editing efficiency.

### SUMMARY OF THE INVENTION

The present disclosure provides CRISPR nucleases that exhibit advantageous enzymatic activities (e.g., nickase activity, high indel activities, high binding activities to the scaffold of a cognate guide RNA, and/or high DNA cleavage activity). The CRISPR nucleases provided herein would be expected to show superior effectiveness when used in gene editing.

Accordingly, provided herein are CRISPR nuclease polypeptides derived from a reference CRISPR nuclease, including Nuclease W (SEQ ID NO: 1), Nuclease S (SEQ ID NO: 49) or Nuclease H (SEQ ID NO: 64). Such CRISPR nucleases comprise a RuvC nuclease domain and an HNH nuclease domain. In some instances, the CRISPR nuclease polypeptides provided herein share an amino acid sequence at least 85% (e.g., at least 90%) identical to the reference CRISPR nuclease provided herein. In some instances, the CRISPR nuclease polypeptides disclosed herein comprise a variant CRISPR nuclease, which comprises at least one mutation relative to the reference CRISPR nuclease.

In some aspects, the present disclosure features CRISPR nuclease polypeptides derived from Nuclease W, gene editing systems comprising the CRISPR nuclease polypeptide derived from Nuclease W, and a guide RNA targeting a genomic site of interest, and uses of the gene editing system for modifying the genomic site of interest in host cells.

In some instances, the CRISPR nuclease polypeptide disclosed herein is an arginine and/or lysine substitution variant of the reference Nuclease W, wherein the variant enzyme exhibits enhanced bioactivities such as indel activities. Alternatively or in addition, the CRISPR nuclease polypeptide may be a nickase variant of the reference Nuclease W, wherein the variant nuclease has reduced or eliminated nuclease activity of one of the nuclease domains relative to the reference Nuclease W, thereby exhibiting nickase activity. In some examples, the engineered CRISPR nuclease polypeptide is a combined arginine/lysine substitution and nickase variant.

In some embodiments, the present disclosure features a CRISPR nuclease polypeptide comprising an amino acid sequence at least 90% identical to SEQ ID NO: 1 (Nuclease W). In some instances, such a CRISPR nuclease polypeptide can be an engineered variant of the reference CRISPR Nuclease W. The reference CRISPR Nuclease W comprises a RuvC nuclease domain and an HNH nuclease domain. Relative to the reference CRISPR Nuclease W, the engineered CRISPR nuclease polypeptide may comprise: (i) one or more mutations in the HNH nuclease domain or in the RuvC nuclease domain that reduce or eliminate the nuclease activity thereof; (ii) one or more arginine and/or lysine substitutions, optionally one or more arginine substitutions; or (iii) a combination of (i) and (ii).

In some instances, the CRISPR nuclease polypeptide derived from Nuclease W comprises one or more mutations in the HNH nuclease domain, for example, at positions E425, H426, and/or N449 relative to SEQ ID NO: 1. In some examples, the mutation is at position E425. In some instances, the one or more mutations in the HNH nuclease domain may be amino acid residue substitutions, e.g., at one or more of positions E425, H426, and N449. In some instances, the mutation at E425 is an amino acid substitution, e.g., E425A, E425G, E425L, or E425S. In some instances, the mutation at H426 is an amino acid substitution, e.g., H426A, H426G, H426L, or H426S. Alternatively or in addition, the mutation at N449 is an amino acid substitution, e.g., N449A, N449G, N449L, or N449S.

In some examples, the CRISPR nuclease polypeptide derived from Nuclease W comprises one or more mutations in the RuvC nuclease domain, for example, at positions D10, E362, and/or D487 relative to SEQ ID NO: 1.

Alternatively or in addition, the engineered CRISPR nuclease polypeptide derived from Nuclease W comprises a bridge helix (BH) domain, a nucleic acid recognition (REC) domain, a phosphate lock loop (PLL) domain, a wedge (WED) domain, and a PAM-interacting (PID) domain. The one or more arginine and/or lysine substitutions, optionally arginine substitutions, are located in the BH domain, in the REC domain, in the PLL domain, in the WED domain, in the PID domain, or a combination thereof. In some embodiments, the engineered CRISPR nuclease polypeptide contains up to 20 arginine and/or lysine substitutions relative to the reference CRISPR nuclease. In some instances, the engineered CRISPR nuclease polypeptide contains up to 15 arginine and/or lysine substitutions relative to the reference CRISPR nuclease. In some examples, the engineered CRISPR nuclease polypeptide may comprise one or more arginine and/or lysine substitutions are at positions A65, V117, A657, N691, S695, S925, and/or E715 in SEQ ID NO: 1.

Any of the CRISPR nuclease polypeptides derived from Nuclease W as disclosed herein may comprise an amino acid sequence at least 90% identical to SEQ ID NO: 1. In some examples, the engineered CRISPR nuclease polypeptide comprises an amino acid sequence at least 95% identical to SEQ ID NO: 1. In other examples, the engineered CRISPR nuclease polypeptide comprises an amino acid sequence at least 98% identical to SEQ ID NO: 1. Any of the engineered CRISPR nuclease polypeptides may comprise the arginine and/or lysine substitutions disclosed herein, any of the mutations in either the HNH or RuvC nuclease domains also disclosed herein, or a combination thereof. In specific examples, the engineered CRISPR nuclease polypeptide may contain the mutations of those listed in Table 5 and Table 6, or a combination thereof.

In some instances, any of the engineered CRISPR nuclease polypeptides derived from Nuclease W as disclosed herein may be a fusion polypeptide further comprising one or more functional elements, which may be heterologous to the CRISPR nuclease moiety in the fusion polypeptide. In some examples, the fusion polypeptide comprises one or more nuclear localization signal (NLS) fused to the CRISPR nuclease moiety at its N-terminus and/or its C-terminus. In some instances, the fusion polypeptide may further comprise one or more peptide linkers connecting an NLS and the CRISPR nuclease moiety in the fusion polypeptide.

Exemplary CRISPR nuclease polypeptides derived from Nuclease W are provided in Table 1 and Table 6, each of which is within the scope of the present disclosure.

In another embodiment, the present disclosure provides a nucleic acid, comprising a nucleotide sequence encoding any of the CRISPR nuclease polypeptides derived from Nuclease W as disclosed herein. In some embodiments, the nucleic acid is an expression vector, in which the nucleotide sequence encoding the engineered CRISPR nuclease polypeptide is in operable linkage to a promoter. Alternatively, the nucleic acid may be a messenger RNA (mRNA). Also within the scope of the present disclosure is a host cell comprising the nucleic acid encoding any of the CRISPR nuclease polypeptides derived from Nuclease W as disclosed herein.

Further, the present disclosure provides a gene editing system, comprising: (a) any of the CRISPR nuclease polypeptides derived from Nuclease W as provided herein or a first nucleic acid encoding the CRISPR nuclease; and (b) a guide RNA (gRNA), or a second nucleic acid encoding the gRNA. The gRNA comprises a scaffold sequence recognizable by the CRISPR nuclease polypeptide derived from Nuclease W and a spacer sequence specific to a target sequence within a genomic site of interest, the target sequence being upstream to a protospacer motif (PAM). The target sequence is upstream to a protospacer motif (PAM). In some embodiments, the PAM is 5′-NRV-3′, in which N represents any nucleotide, R represents A or G, and V represents A, C, or G. In some examples, the PAM is 5′-NAV-3′, in which N is any nucleotide and V represents A, C, or G. In some examples, the PAM is 5′-NRR′3′, for example, 5′-CAG-3′. In other examples, the PAM is 5′-RRR-3′, for example, 5′-AAA-3′, or 5′-GAG-3′.

In some instances, the scaffold sequence comprises capable of being utilized by a CRISPR nuclease polypeptide derived from Nuclease W as disclosed herein comprises a nucleotide sequence at least 85% identical to SEQ ID NO: 2. In some instances, the scaffold comprises one or more deletions, one or more nucleotide substitutions, or a combination thereof, as compared with SEQ ID NO: 2. In other embodiments, the scaffold comprises SEQ ID NO: 2.

In another aspect, the present disclosure provides CRISPR nuclease polypeptides derived from reference Nuclease S, comprising a RuvC nuclease domain and a HNH nuclease domain. In some instances, the CRISPR nuclease polypeptide derived from Nuclease S may comprise an amino acid sequence at least 90% identical to SEQ ID NO: 49 (Nuclease S). In some examples, the CRISPR nuclease may comprise an amino acid sequence at least 95% identical to SEQ ID NO: 49. In specific examples, the CRISPR nuclease may comprise an amino acid sequence at least 98% identical to SEQ ID NO: 49. In some instances, the CRISPR nuclease polypeptide may have enhanced enzymatic activity as compared with the reference CRISPR nuclease.

In some embodiments, the CRISPR nuclease polypeptide derived from Nuclease S may be an engineered variant thereof, which may comprise one or more arginine and/or lysine substitutions (e.g., one or more arginine substitutions) relative to the reference CRISPR nuclease. In some instances, the engineered CRISPR nuclease polypeptide may further comprise a bridge helix (BH) domain, a wedge (WED) domain, and a PAM-interacting (PID) domain. In some instances, the one or more arginine and/or lysine substitutions (e.g., arginine substitutions) are located in the BH domain, in the REC domain, in the HNH nuclease domain, in the PID domain, or a combination thereof. In specific examples, the one or more arginine and/or lysine substitutions (e.g., arginine substitutions) are located at one or more of positions T72, N76, D179, Q200, E277, H280, I322, V324, V504, W509, D844, and I845 in SEQ ID NO: 49. In some examples, the one or more arginine and/or lysine substitutions (e.g., arginine substitutions) may be at the following positions relative to SEQ ID NO: 49: a) W509, T72, and I845; b) H280, T72, and I845; c) W509 and I845; d) H280, W509, T72, and I845; e) D844 and W509; f) W509 and T72; g) D844, W509, and I845; h) H280, W509, and I845; or i) D844 and H280. In some specific examples, the one or more arginine and/or lysine substitutions (e.g., arginine substitutions) may be at the positions of W509, T72, and I845 of SEQ ID NO: 49. In other specific examples, the one or more arginine and/or lysine substitutions (e.g., arginine substitutions) may be at the positions of H280, T72, and I845 of SEQ ID NO: 49.

In some examples, the engineered CRISPR nuclease polypeptide derived from Nuclease S may comprise the following arginine substitution combinations: a) W509R, T72R, I845R; b) H280R, T72R, I845R; c) W509R, I845R; d) H280R, W509R, T72R, I845R; e) D844R, W509R; f) W509R, T72R; g) D844R, W509R, I845R; h) H280R, W509R, I845R; or i) D844R, H280R. In some specific examples, the engineered variant of CRISPR Nuclease S may comprise the arginine substitutions of W509R, T72R, and I845R. In other specific examples, the engineered CRISPR nuclease polypeptide may comprise the arginine substitutions of H280R, T72R, and I845R.

Any of the engineered CRISPR nuclease polypeptides derived from Nuclease S as disclosed herein may contain up to 20 arginine and/or lysine substitutions (e.g., up to 20 arginine substitutions), for example, up to 15 arginine and/or lysine substitutions (e.g., up to 15 arginine substitutions).

In some instances, the engineered CRISPR nuclease polypeptide derived from Nuclease S as provided herein may comprise one or more mutations leading to nickase activity, for example, one or more mutations at position E447, H448, and/or N471 in SEQ ID NO: 49, or one or more mutations at position D25, E371, or D604 in SEQ ID NO: 49. In some examples, the mutation can be an amino acid substitution of H448A. In specific examples, the engineered CRISPR nuclease polypeptide may comprise arginine and/or lysine substitutions (e.g., arginine substitutions) at positions H280, T72, and I845 of SEQ ID NO: 49 or at positions W509R, T72R, and I845R of SEQ ID NO: 49 and further comprises the amino acid substitution of H448A.

In some instances, the CRISPR nuclease polypeptide derived from Nuclease S as disclosed herein may be a fusion polypeptide comprising any of the CRISPR nuclease derived from Nuclease S and one or more additional fragments, which may be heterologous to the CRISPR nuclease moiety in the fusion polypeptide. In some instances, the one or more additional fragments may comprise one or more NLSs. Such NLS sites may be located at the N-terminus, at the C-terminus, or both. In some instances, the fusion polypeptide may further comprise one or more peptide linkers connecting an NLS and the CRISPR nuclease moiety in the fusion polypeptide.

Also provided herein is a nucleic acid, comprising a nucleotide sequence encoding any of the CRISPR nuclease polypeptides derived from Nuclease S disclosed herein. In some instances, the nucleic acid is an expression vector, in which the nucleotide sequence encoding the CRISPR nuclease polypeptide is in operable linkage to a promoter. Further, the present disclosure provides a host cell comprising the nucleic acid coding for the CRISPR nuclease polypeptide as disclosed herein.

In another embodiment, the present disclosure features a gene editing system, comprising: (a) any of the CRISPR nuclease polypeptide derived from Nuclease S disclosed herein or a first nucleic acid encoding the CRISPR nuclease; and (b) a guide RNA (gRNA), or a second nucleic acid encoding the gRNA. The gRNA comprises a scaffold sequence recognizable by the CRISPR nuclease polypeptide and a spacer, which is specific to a target sequence within a genomic site of interest. The target sequence is upstream to a protospacer adjacent motif (PAM).

In some embodiments, the scaffold sequence capable of being utilized by a CRISPR nuclease polypeptide derived from Nuclease S as disclosed herein may comprise a nucleotide sequence at least 85% identical to SEQ ID NO: 63. Alternatively or in addition, the scaffold sequence may be a fragment of SEQ ID NO: 63. In some instances, the scaffold sequence comprises one or more deletions, one or more nucleotide substitutions, or a combination thereof, as compared with SEQ ID NO: 63.

In some embodiments, the target sequence is adjacent to a PAM of 5′-NRNC-3′ (e.g., 5′-NRCC-3′ or 5′-ARCC-3′) or 5′-NRYM-3′, in which N represents any nucleotide, R represents A or G, Y represents C or T, and M represents A or C.

In yet another aspect, the present disclosure provides a CRISPR nuclease polypeptide derived from Nuclease H (SEQ ID NO: 64), comprising a RuvC nuclease domain and an HNH nuclease domain. In addition to the nuclease domains, the CRISPR nuclease polypeptide may comprise a bridge helix (BH) domain, a nucleic acid recognition (REC) domain, a phosphate lock loop (PLL) domain, a wedge (WED) domain, and a PAM-interacting (PID) domain.

In some embodiments, the CRISPR nuclease polypeptide derived from Nuclease H comprises an amino acid sequence at least 90% identical to SEQ ID NO: 64. In some examples, the CRISPR nuclease polypeptide comprises an amino acid sequence at least 95% identical to SEQ ID NO: 64. In other examples, the CRISPR nuclease polypeptide comprises an amino acid sequence at least 98% identical to SEQ ID NO: 64. In some instances, the engineered CRISPR nuclease polypeptide may have enhanced enzymatic activity as compared with the reference CRISPR nuclease.

In some instances, the CRISPR nuclease polypeptide is an engineered variant of Nuclease H, which may comprise at least one mutation relative to CRISPR Nuclease H set forth as SEQ ID NO: 64. In some instances, the at least one mutation may comprise one or more arginine substitutions, lysine substitutions, or a combination thereof, relative to a reference CRISPR nuclease, which is set forth as SEQ ID NO: 64 (Nuclease H). In some embodiments, the at least one mutation can be located in one or more of the BH domain, the REC domain, the PLL domain, the WED domain, the PID domain, and a nuclease domain.

In some instances, the engineered CRISPR nuclease polypeptide derived from Nuclease H as disclosed herein may contain up to 20 arginine/lysine substitutions (e.g., arginine substitutions) relative to the reference CRISPR nuclease. For example, the engineered CRISPR nuclease polypeptide contains up to 15 arginine/lysine substitutions (e.g., arginine substitutions), for example, up to 12 arginine substitutions, relative to the reference CRISPR nuclease.

In some examples, the arginine and/or lysine substitutions (e.g., arginine substitutions) may be at one or more of positions L54, L323, D912, T783, E43, S680, and A170 in SEQ ID NO: 64. For example, the engineered CRISPR nuclease polypeptide may comprise arginine substitutions of L54R, L323R, D912R, T783R, E43R, S680R, A170R, or a combination thereof. In specific examples, the engineered CRISPR nuclease polypeptide may comprise a combination of arginine/lysine substitutions relative to the reference CRISPR Nuclease H. In one instance, the engineered CRISPR nuclease polypeptide may comprise arginine/lysine substitutions at positions E43, A170, and T783 (e.g., E43R, A170R, and T783R). In another instance, the engineered CRISPR nuclease polypeptide may comprise arginine/lysine substitutions at positions L54, L323, and T783 (e.g., L54R, L323R, and T783R).

Alternatively or in addition, the CRISPR nuclease polypeptide derived from Nuclease H may comprises the one or more nickase mutations of (b). In some examples, the one or more nickase mutations may be located at D7, R329, H338, E356, E419, H420, N443, and/or D580 of SEQ ID NO: 64.

Any of the CRISPR nuclease polypeptide derived from Nuclease H as disclosed herein may be a fusion polypeptide further comprising one or more functional fragments, which may be heterologous to the CRISPR nuclease moiety in the fusion polypeptide. In some instances, the one or more functional fragments comprise one or more NLSs. Such NLS sites may be located at the N-terminus, at the C-terminus, or both of the fusion polypeptide. In some instances, the fusion polypeptide may further comprise one or more peptide linker connecting an NLS and the CRISPR nuclease moiety in the fusion polypeptide.

Also provided herein is a nucleic acid, comprising a nucleotide sequence encoding any of the CRISPR nuclease polypeptides derived from Nuclease H as disclosed herein. In some instances, the nucleic acid is an expression vector, in which the nucleotide sequence encoding the CRISPR nuclease polypeptide is in operable linkage to a promoter. Alternatively, the nucleic acid may be a messenger RNA (mRNA) molecule. Further, the present disclosure provides a host cell comprising the nucleic acid coding for the CRISPR nuclease polypeptide or the fusion polypeptide as disclosed herein.

In another aspect, the present disclosure features a gene editing system, comprising: (a) any of the CRISPR nuclease polypeptides derived from Nuclease H as disclosed herein or a first nucleic acid encoding such; and (b) a guide RNA (gRNA), or a second nucleic acid encoding the gRNA. The gRNA comprises a scaffold sequence recognizable by the CRISPR nuclease polypeptide and a spacer, which is specific to a target sequence specific to a genomic site of interest. The target sequence is upstream to a PAM.

In some embodiments, the scaffold sequence capable of being utilized by a CRISPR nuclease polypeptide derived from Nuclease H as disclosed herein may comprise a nucleotide sequence at least 85% identical to SEQ ID NO: 77. Alternatively or in addition, the scaffold sequence may be a fragment of SEQ ID NO: 77. In some instances, the scaffold sequence may comprise one or more deletions, one or more nucleotide substitutions, or a combination thereof, as compared with SEQ ID NO: 77.

In some embodiments, the target sequence is adjacent to a PAM of 5′-NRM-3′ (e.g., 5′-NRC-3′) or 5′-NRMY-3′ (e.g., 5′-NRCY-3′), in which N represents any nucleotide, R represents A or G, M represents A or C, and Y represents C.

Any of the gene editing systems provided herein may further comprise one or more lipid excipients, which are associated with the CRISPR nuclease polypeptide (or coding nucleic acid) and/or the gRNA (or coding nucleic acid). In some instances, the one or more lipid excipients form lipid nanoparticles (LNPs) associated with or encapsulating the CRISPR nuclease polypeptide (or coding nucleic acid) and/or the gRNA (or coding nucleic acid).

In addition, the present disclosure provides a gene editing method, comprising delivering any of the gene editing systems as provided herein to a host cell to edit a genomic site targeted by the gRNA of the gene editing system. In some embodiments, the host cell is cultured in vitro. In other embodiments, the host cell is located in a subject who needs the gene editing.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing percentages of NGS reads comprising indels at six genetic loci as indicated in the presence or absence of the CRISPR Nuclease W of SEQ ID NO: 1.

FIGS. 2A-2D include gel images and quantification of nuclease activity by Nuclease W and variants thereof. FIG. 2A: Gel image captured using an 800 nm channel showing in vitro cleavage of the target strand (labelled on the 5′ end with an IR800 dye) of the target DNA substrate by Nuclease W, putative HNH-knockout nickase variants or putative RuvC-knockout nickase variants of Nuclease W. FIG. 2B: Gel image captured using a 700 nm channel showing in vitro cleavage of the non-target strand (labelled on the 5′ end with an IR700 dye) of a target DNA substrate by Nuclease W, putative HNH-knockout nickase variants or putative RuvC-knockout nickase variants of Nuclease W. FIG. 2C: Overlaid images captured using 800 nm and 700 nm channels of FIG. 2A and FIG. 2B. FIG. 2D: Quantification of the percent of cleaved target and non-target DNA generated by Nuclease W, putative HNH-knockout nickase variants or putative RuvC-knockout nickase variants of Nuclease W tested.

FIG. 3 is a diagram showing percentages of NGS reads comprising indels at six genetic loci as indicated in the presence or absence of the CRISPR Nuclease S of SEQ ID NO: 49.

FIGS. 4A-4D include gel images and quantification of nuclease activity of Nuclease S and variants thereof. FIG. 4A: Gel image captured using an 800 nm channel showing in vitro cleavage of the target strand (labelled on the 5′ end with an IR800 dye) of the target DNA substrate by Nuclease S, putative HNH-knockout nickase variants of Nuclease S, or putative RuvC-knockout nickase variants of Nuclease S. FIG. 4B: Gel image captured using a 700 nm channel showing in vitro cleavage of the non-target strand (labelled on the 5′ end with an IR700 dye) of a target DNA substrate by Nuclease S, putative HNH-knockout nickase variants of Nuclease S, or putative RuvC-knockout nickase variants of Nuclease S. FIG. 4C: Overlaid images captured using 800 nm and 700 nm channels of FIG. 4A and FIG. 4B. FIG. 4D: Quantification of the percent of cleaved target and non-target DNA generated by Nuclease S, putative HNH-knockout nickase variants of Nuclease S, or putative RuvC-knockout nickase variants of Nuclease S tested.

FIG. 5 is a diagram showing percentages of NGS reads comprising indels at six genetic loci as indicated in the presence or absence of the CRISPR Nuclease H of SEQ ID NO: 64.

### DETAILED DESCRIPTION OF THE INVENTION

Provided herein are CRISPR nuclease polypeptides derived from a reference CRISPR nuclease, including Nuclease W (SEQ ID NO: 1), Nuclease S (SEQ ID NO: 49) or Nuclease H (SEQ ID NO: 64). Such a CRISPR nuclease polypeptide may comprise the reference nuclease or a variant thereof as disclosed herein. In some instances, the CRISPR nuclease polypeptides provided herein may be a variant nuclease (a.k.a., engineered nuclease) of the reference nuclease comprising at least one mutation relative to the reference nuclease.

The variant CRISPR nuclease polypeptides may comprise one or more mutations (e.g., arginine and/or lysine substitutions) relative to the reference CRISPR nuclease. Alternatively or in addition, the variant CRISPR nuclease polypeptide may comprise one or more mutations in either the RuvC nuclease domain or the HNH nuclease domain. Such mutations (i.e., nickase mutations) may reduce or eliminate the nuclease activity of either the RuvC or the HNH nuclease domain, leading to a variant exhibiting nickase activity. As used herein, the term “nickase” refers to an enzyme that cuts one strand of a double-stranded DNA at a specific recognition nucleotide sequence (e.g., the target sequence disclosed herein). A nickase may interact with one strand of the DNA duplex to produce DNA molecules that are cut at one strand (a.k.a., nicked). In some embodiments, a nickase is a variant of a CRISPR nuclease that comprises a deactivated HNH domain. In some embodiments, a nickase is a variant of a CRISPR nuclease that comprises a deactivated RuvC domain.

Any of the variant CRISPR nuclease polypeptides provided herein may share a high sequence homology relative to the reference CRISPR Nuclease W (SEQ ID NO: 1), Nuclease S (SEQ ID NO: 49) or Nuclease H (SEQ ID NO: 64) (e.g., at least 85% sequence identity, for example, at least 90%, at least 95%, at least 97%, at least 98%, at least 99% or higher sequence identity).

The variant CRISPR nuclease polypeptides provided herein are expected to possess advantageous features relative to the reference CRISPR nuclease, for example, exhibiting nickase activity and/or higher nuclease activity, higher indel activity, etc. As such, the variant CRISPR nuclease polypeptides disclosed herein would be expected to exhibit improved gene editing relative to the reference CRISPR nuclease, e.g., nickase activity, higher efficiency and accuracy in gene editing involving strand replacement.

In some instances, the variant CRISPR nuclease polypeptides may be fusion polypeptides comprising a CRISPR nuclease (e.g., SEQ ID NO: 1, SEQ ID NO: 49, or SEQ ID NO: 64, or a variant thereof) (nuclease moiety) and one or more additional functional fragments such as those described herein (e.g., nuclease localization signal or NLS, and/or peptide linkers). In addition to the advantageous features noted above, the fusion polypeptides possess additional functions attributable to the fusion partners.

Accordingly, the present disclosure provides CRISPR nuclease polypeptides derived from the reference CRISPR nuclease (Nuclease W, Nuclease S, or Nuclease H), gene editing systems comprising such, and gene editing methods using such.

### I. CRISPR Nuclease Polypeptides

As used herein, the term “CRISPR nuclease” refers to an RNA-guided effector that is capable of binding a nucleic acid and introducing a single-stranded break or double-stranded break. A CRISPR nuclease typically comprises multiple functional domains, e.g., nuclease domains (e.g., RuvC and/or HNH), PLMP domain, bridge helix (BH) domain, nucleic acid recognition (REC) domain, phosphate lock loop (PLL), wedge domain (WED), PAM-interacting domain (PID), or a combination thereof. As used herein, the term “domain” refers to a distinct functional and/or structural unit of a polypeptide. In some instances, a functional domain may be linear. In other instances, a functional domain can be discontinuous and conformational. In some embodiments, a domain may comprise a conserved amino acid sequence.

As used herein, the term “variant CRISPR nuclease polypeptide” refers to a CRISPR nuclease polypeptide comprising an alteration, e.g., a substitution, insertion, deletion and/or fusion, at one or more residue positions, compared to the reference CRISPR nuclease such as Nuclease W, Nuclease S, or Nuclease H provided herein.

The variant CRISPR nuclease polypeptides provided herein are expected to exhibit one or more modulated activities (e.g., enhanced or reduced) relative to the reference CRISPR nuclease. As used herein, the term “activity” refers to a biological activity. In some embodiments, activity includes enzymatic activity, e.g., catalytic ability of an effector. For example, activity can include nuclease activity. In some embodiments, activity includes binding activity, e.g., binding of an effector (e.g., a CRISPR nuclease) to an RNA guide and/or target nucleic acid. In some examples, the variant CRISPR nuclease polypeptides disclosed herein have an enhanced binding to a cognate guide RNA (gRNA) as compared with the reference CRISPR nuclease, e.g., having a binding activity at least 20%, 30%, 40%, 50%, 60%, 70%, 80%, 90%, 100%, 2-fold, 2-fold, 5-fold, 10-fold, or greater than that of the reference CRISPR nuclease. A cognate gRNA refers to a gRNA having a scaffold recognizable by the CRISPR nuclease.

In some examples, the variant CRISPR nuclease polypeptides disclosed herein have an enhanced enzymatic activity relative to the reference CRISPR nuclease, e.g., having an enzymatic activity at least 20%, 30%, 40%, 50%, 60%, 70%, 80%, 90%, 100%, 2-fold, 2-fold, 5-fold, 10-fold, or greater than that of the reference CRISPR nuclease. In other examples, the variant CRISPR nuclease polypeptides disclosed herein have a decreased enzymatic activity relative to the reference CRISPR nuclease, e.g., having an enzymatic activity at least 20%, 30%, 40%, 50%, 60%, or 70% lower than that of the reference CRISPR nuclease. In some instances, the decreased enzymatic activity is achieved by reducing or diminishing the nuclease activity of the RuvC domain. In some instances, the decreased enzymatic activity is achieved by reducing or diminishing the nuclease activity of the HNH domain.

In some instances, the variant CRISPR nuclease polypeptides disclosed herein have enhanced indel activity relative to the reference CRISPR nuclease. As used herein, the term “indel activity” refers to the ability of a CRISPR nuclease to introduce an indel (insertion/deletion) into a sequence (e.g., a genomic target).

In some embodiments, the variant CRISPR nuclease polypeptide provided herein share a high sequence homology relative to the reference CRISPR nuclease. For example, the variant CRISPR nuclease polypeptide may comprise an amino acid sequence at least 70% (e.g., at least 80%, 85%, 90%, 95%, or higher) identical to SEQ ID NO: 1, SEQ ID NO: 49, or SEQ ID NO: 64. In some instances, the variant CRISPR nuclease polypeptide may comprise an amino acid sequence at least 90% identical to SEQ ID NO: 1, SEQ ID NO: 49, or SEQ ID NO: 64. In some instances, the variant CRISPR nuclease polypeptide may comprise an amino acid sequence at least 95% identical to SEQ ID NO: 1, SEQ ID NO: 49, or SEQ ID NO: 64. In other instances, the variant CRISPR nuclease polypeptide may comprise an amino acid sequence at least 97% (e.g., 98%, 99%, 99.5%, or greater) identical to SEQ ID NO: 1, SEQ ID NO: 49, or SEQ ID NO: 64.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some instances, the variant CRISPR nuclease polypeptide disclosed herein may comprise one or more arginine and/or lysine substitutions relative to the reference Nuclease W, Nuclease S, or Nuclease H provided herein. “Arginine substitutions” or “lysine substitution” refers to the replacement of a non-arginine or non-lysine residue in the reference nuclease with an arginine or lysine residue.

In some instances, one or more of the substituting arginine residues may be replaced by a conservative amino acid residue such as lysine or histidine. In some embodiments, the variant CRISPR nuclease polypeptide provided herein may comprise one or more arginine substitutions, one or more lysine substitutions, or a combination thereof.

In some instances, a variant CRISPR nuclease polypeptide provided herein may contain one or more conservative amino acid residue substitutions, either taken alone or in combination with the other types of mutations disclosed herein.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### (A) Nuclease W and Engineered Variants Thereof

In some embodiments, the CRISPR nuclease polypeptide is derived from Nuclease W, including both the wild-type Nuclease W (SEQ ID NO: 1), a variant thereof such as those disclosed herein, or a fusion polypeptide comprising such. The variant CRISPR nuclease polypeptides of Nuclease W may be produced via introducing one or more mutations to the reference CRISPR nuclease to modulate (e.g., enhance or reduce) one or more activities of the nuclease.

The reference CRISPR nuclease of SEQ ID NO: 1 (Nuclease W; see Table 1 below) is a CRISPR nuclease that comprises both a RuvC nuclease domain (located at residues 1-39, 323-363, and 530-655 of SEQ ID NO: 1) and a HNH domain (located at residues 364-529 of SEQ ID NO: 1). The RuvC nuclease domain and the HNH nuclease domain coordinate cleavage of the DNA strand adjacent to the 5′-NDR-3′ motif (e.g., 5′-NRG-3′ or 5′-NRR′-3′ such as 5′-NGG-3′ or 5′-RRR-3′, or 5′-NAV-3′), in which N represents any nucleotide, D represents A, G, or T, R represents G or A, and V represents A, C, or G. Positions D10, E362, and D587 are deemed the active sites in the RuvC domain, and positions E425, H426, and N449 are deemed the active sites in the HNH domain. R329 and H338 may also be important for the nuclease activity of the RuvC domain. In addition to the nuclease domains, the reference CRISPR Nuclease W of SEQ ID NO: 1 also includes a BH domain (residues 40-78 of SEQ ID NO: 1), a REC domain (residues 79-322 of SEQ ID NO: 1), a PLL domain (residues 656-670), a WED domain (residues 671-819), and a PID domain (residues 820-946).

Compared to the CRISPR-Cas9 nuclease from Streptococcus pyogenes, the reference CRISPR Nuclease W of SEQ ID NO: 1 disclosed herein is smaller. The scaffold utilized by the reference CRISPR nuclease of SEQ ID NO: 1 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller CRISPR nuclease size. These features would be beneficial for delivery. Arginine substitutions can be introduced into the CRISPR Nuclease W of SEQ ID NO: 1 to increase indel activity.

Nuclease W recognizes different PAM motifs in genomic targets as compared with SpCas9, allowing for additional gene targets to be edited. Further, the RuvC domain of Nuclease W cleaves the non-target strand within 3 to 8 nucleotides upstream of the PAM, and the HNH cleaves the target strand between 3 and 4 nucleotides upstream of the PAM, each leading to a cut site with an overhang of 0-4 nucleotides (most commonly a 2- or 3-nucleotide overhang). Differently, SpCas9 RuvC cleaves the non-target strand within 3 to 5 nucleotides upstream of the PAM by its RuvC domain and cleaves the target strand between 3 and 4 nucleotides upstream of the PAM by its HNH domain, leading to cut sites with an overhang of 0-3 nucleotides (mostly commonly a blunt cut).

Additionally, use of gene editing systems comprising Nuclease W or variants thereof can result in the introduction of indels into a target nucleic acid that are larger than those capable of being introduced by SpCas9. For example, insertions induced by use of Nuclease W or variants thereof can range from about 1-nucleotide to about 30-nucleotides (most commonly about 3-nucleotides). Deletions induced by use of Nuclease W or variants thereof can range from about 1-nucleotide to about 25-nucleotides or larger (most commonly about 11-nucleotides). Further, since the reference CRISPR Nuclease W of SEQ ID NO: 1 comprises a RuvC domain and an HNH domain, nickase variants can be engineered, e.g., via disrupting nuclease activity of either the RuvC domain or the HNH domain.

The variant CRISPR nuclease polypeptides provided herein are derived from the reference CRISPR Nuclease W of SEQ ID NO: 1, e.g., via introducing one or more mutations to the reference CRISPR nuclease to modulate (e.g., enhance or reduce) one or more activities of the nuclease.

In some embodiments, the variant CRISPR nuclease polypeptides provided herein, relative to the reference CRISPR Nuclease W of SEQ ID NO: 1, comprises one or more mutations in either the RuvC nuclease domain or the HNH nuclease domain (e.g., in the HNH nuclease domain) to reduce or eliminate the nuclease activity, and/or comprises one or more arginine and/or lysine substitutions to improve nuclease features suitable for use in gene editing.

The variant CRISPR nuclease polypeptide provided herein can contain one or more alterations relative to the reference CRISPR Nuclease W of SEQ ID NO: 1, e.g., one or more amino acid residue substitutions, one or more deletions, one or more insertions, one or more fusions, or a combination thereof. In some instances, the alterations may be introduced into the BH domain, the REC domain, the PLL domain, the WED domain, the PID domain, or a combination thereof. In some instances, no alterations are introduced into the RuvC and/or the HNH nuclease domains, or at the active sites and/or sites involved in activity of these domains as provided herein.

In some embodiments, the variant CRISPR nuclease polypeptide provided herein may comprise one or more arginine and/or lysine substitutions relative to SEQ ID NO: 1. In some examples, the variant CRISPR nuclease polypeptide may contain up to 20 arginine and/or lysine substitutions, e.g., up to 19, 18, 17, 16, 15, 14, 13, 12, 11, 10, 9, 8, 7, 6, 5, 4, 3, or 2 arginine and/or lysine substitutions. In specific examples, the variant CRISPR nuclease polypeptide may contain 12, 11, 10, 9, 8, 7, 6, 5, 4, 3, or 2 arginine and/or lysine substitutions.

In some instances, one or more of the substituting arginine residues may be replaced by a conservative amino acid residue such as lysine or histidine. In some embodiments, the variant CRISPR nuclease polypeptide derived from Nuclease W provided herein may comprise one or more arginine substitutions, one or more lysine substitutions, or a combination thereof.

In some instances, the arginine substitutions may be located in the BH domain, in the PLL domain, in the WED domain, in the PID domain, or in any of the combination thereof. In some examples, the variant CRISPR nuclease polypeptide may contain one or more arginine and/or lysine substitutions at one or more of positions: K14, L27, L37, D39, S44, K45, E48, V51, M56, T59, K60, K64, A65, A68, H69, E71, L75, S76, V84, V85, V88, 102, P106, 108, E109, E112, E113, E114, A115, E116, F118, F120, E124, S127, N131, L134, E135, E139, L142, I143, H151, D156, A173, A177, D189, 206, S210, D211, L214, V235, S238, Q245, L248, F260, S262, G263, L264, A265, Q266, D267, L268, P269, E270, E271, T272, 1273, E274, T280, N288, E291, Q292, S293, G294, E277, A305, Y309, G314, T324, S326, A380, D382, E386, Q389, Q390, Y394, G395, K397, 400, K405, N408, K416, P417, E419, T420, N432, K455, A456, S461, D475, D447, A460, S468, A478, D479, L499, S457, K480, K482, T483, E518, G521, V523, G529, P532, Q543, V548, Q564, V577, G579, T581, T599, 602, A603, N605, W609, I610, S612, A613, V615, K617, S620, A621, Q624, Q627, A632, H635, Q648, H665, S666, L679, E680, A684, A688, A690, A694, N698, K701, E702, K703, P705, D707, K710, E711, V717, V718, A726, T733, E739, S740, A742, A743, I746, S747, K751, D759, S764, Q769, A770, A772, D777, D780, G783, E784, A785, A788, I794, A801, Q811, Q814, Q826, L833, G834, S835, K843, A854, V856, P857, G858, F859, A861, S863, E864, Q871, H890, A891, Q894, S895, A896, E897, 905, H911, K930, G947, I950, P951, and I953 relative to SEQ ID NO: 1.

In some examples, the arginine and/or lysine substitutions are at positions A65, V117, A657, S695, N691, E715, and/or S925 relative to SEQ ID NO: 1. For example, the engineered CRISPR nuclease polypeptide may comprise A65R, V117R, A657R, S695R, N691R, E715R, and/or S925R relative to SEQ ID NO: 1. Alternatively, for example, the engineered CRISPR nuclease polypeptide may comprise A65K, V117K, A657K, S695K, N691K, E715K, and/or S925K relative to SEQ ID NO: 1.

In some instances, the variant CRISPR nuclease polypeptide may comprise a combination of at least two arginine and/or lysine substitutions (e.g., 2 or 3) relative to the reference Nuclease W of SEQ ID NO: 1. For example, the substitutions may be at two or more positions of A65, V117, A657, S695, N691, E715, and S925. See Table 5 below, each of which is within the scope of the present disclosure. Specific examples include: (1) A65, E715, and S925; (2) A65, A657 and S925; (3) A657, S695, and S925; (4) N691, S695, and S925; (5) A65, N691, and S925; (6) V117, S695, and S925; (7) N691, E715, and S925; (8) V117, E715, and S925; (9) S695, E715, and S925; (10) A65, S695, and S925; (11) A657, N691, and S925; (12) A657, N691, and S695; (13) A65, V117, and E715; (14) V117, A657, and S925; (15) V117, S695, and E715; (16) S695, and S925; (17) A65, A657, and E715; and (18) E715 and S925. See also Example 3 below. In one specific example, the arginine and/or lysine (optionally arginine) substitutions are at positions A65, A657 and S925 relative to SEQ ID NO: 1; e.g., A65R, A657R, and S925R.

Alternatively or in addition, the variant CRISPR nuclease polypeptide derived from Nuclease W provided herein may comprise one or more mutations within either the RuvC or the HNH nuclease domain to reduce or eliminate the nuclease activity of the target domain, thereby producing a variant with nickase activity. Such mutations (nickase mutations) may be deletions, insertions, amino acid substitutions, or a combination thereof. In some embodiments, the mutations within either the RuvC or the HNH nuclease domain are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R. Groups of conservative amino acid residue substitutions are provided herein.

In some instances, the one or more mutation may be within the HNH nuclease domain, for example, at E425, H426, and/or N449 of SEQ ID NO: 1. In some examples, the mutations may be amino acid residue substitutions and the native amino acid residues in SEQ ID NO: 1 may be replaced by an amino acid residue not of the same type as the native residues. For example, a positively charged residue may be replaced by a non-charged amino acid residue, or vice versa. In some examples, the amino acid residue substitution at E425 may be E425G, E425A, E425L, or E425S. In one specific example, the mutation can be D844A. In another example, the amino acid residue substitution at H426 may be H426G, H426A, H426I, H426L, H426M, H426V, or H426S. In one specific example, the mutation at position H426 can be H426A. Alternatively or in addition, the amino acid residue substitution may be at position N449, for example, N449G, N449A, N449L, or N449S. In one example, the mutation at position N449 is N449A.

In some instances, the one or more mutation may be with the RuvC nuclease domain, for example, at position D10, E362, D487, or a combination thereof, of SEQ ID NO: 1 (e.g., at position E362 and/or D487). In some examples, the mutations may be amino acid residue substitutions and the native amino acid residues in SEQ ID NO: 1 may be replaced by an amino acid residue not of the same type as the native residues. For example, a positively charged residue may be replaced by a non-charged amino acid residue, or vice versa. In some examples, the amino acid residue substitution at D10 may be D10G, D10A, D10L, or D10S. In some examples, the amino acid residue substitution at E362 may be E362G, E362A, E362L, or E362S. Alternatively or in addition, the amino acid residue substitution at D487 may be D487G, D487A, D487L, or D487S.

In some examples, the variant CRISPR nuclease polypeptide derived from Nuclease W provided herein may be a nickase variant, which comprises one or more mutations in one nuclease domain (e.g., the HNH nuclease domain such as at position E425, e.g., E425A). Exemplary nickase variants are provided in Table 6 below, each of which is within the scope of the present disclosure.

In some examples, the variant CRISPR nuclease polypeptide derived from Nuclease W provided herein may comprise both one or more arginine/lysine substitutions (e.g., arginine substitutions) such as those disclosed herein and one or more nickase mutations as also disclosed herein. For example, any of the nickase variants disclosed above may further comprise one or more arginine or lysine substitutions (e.g., arginine substitutions) for enhancing certain features, such as indel activities. Exemplary arginine and/or lysine substitutions are provided herein, for example, at one or more of positions A65, V117, A657, 5695, N691, E715, and/or S925 relative to SEQ ID NO: 1 (e.g., at positions A65, A657 and S925 in SEQ ID NO: 1).

### (B) Nuclease S and Engineered Variants Thereof

In some embodiments, the CRISPR nuclease polypeptide is derived from Nuclease S, including both the wild-type Nuclease S (SEQ ID NO: 49), a variant thereof such as those disclosed herein, or a fusion polypeptide comprising such. The variant CRISPR nuclease polypeptides of Nuclease S may be produced via introducing one or more mutations to the reference CRISPR nuclease to modulate (e.g., enhance or reduce) one or more activities of the nuclease.

The reference CRISPR nuclease of SEQ ID NO: 49 (Nuclease S; see Table 9 below) is a CRISPR nuclease that comprises both a RuvC nuclease domain (located at residues 1-59, 336-374, 546-676 of SEQ ID NO: 49) and a HNH domain (located at residues 375-545 of SEQ ID NO: 49). The RuvC nuclease domain and the HNH nuclease domain coordinate cleavage of the DNA strand adjacent to 5′-NRCC-3′, 5′-NRNC-3′, 5′NRYM-3′, or 5′-ARCC-3′ PAM motif, in which N represents any nucleotide, R represents A or G, Y represents C or T, and M represents A or C. Positions D25, E371 and D604 are deemed the active sites in the RuvC domain, and positions E447, H448, and N471 are deemed the active sites in the HNH domain. R580 and H601 may also be important for the nuclease activity of the RuvC domain. In addition to the nuclease domains, the reference CRISPR Nuclease S of SEQ ID NO: 49 also includes a BH domain (residues 60-92 of SEQ ID NO: 49), a REC domain (residues 93-335 of SEQ ID NO: 49), a PLL domain (residues 677-695 of SEQ ID NO: 49), a WED domain (residues 696-843 of SEQ ID NO: 49), and a PID domain (residues 844-992 of SEQ ID NO: 49).

Compared to the CRISPR-Cas9 nuclease from Streptococcus pyogenes, the reference CRISPR Nuclease S of SEQ ID NO: 49 disclosed herein is smaller. The scaffold utilized by the reference CRISPR nuclease of SEQ ID NO: 49 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller CRISPR nuclease size. These features would be beneficial for delivery. Arginine and/or lysine substitutions can be introduced into the reference CRISPR Nuclease S of SEQ ID NO: 49 to increase indel activity.

Nuclease S recognizes different PAM motifs in genomic targets as compared with SpCas9, allowing for additional gene targets to be edited. Further, the RuvC domain of Nuclease S cleaves the non-target strand within 4 to 6 nucleotides upstream of the PAM, and the HNH cleaves the target strand between 3 and 4 nucleotides upstream of the PAM, each leading to a cut site with an overhang of 1-3 nucleotides (most commonly a 3 nucleotide overhang). This cutting pattern is different from that of SpCas9 described above.

Additionally, use of gene editing systems comprising Nuclease S or variants thereof can result in the introduction of indels into a target nucleic acid that are larger than those capable of being introduced by SpCas9. For example, insertions induced by use of Nuclease S or variants thereof can range from about 1-nucleotide to about 10-nucleotides (most commonly about 3-nucleotides). Deletions induced by use of Nuclease S or variants thereof can range from about 1-nucleotide to about 30-nucleotides or larger (most commonly about 3-nucleotides or about 13-nucleotides). Further, since the reference CRISPR Nuclease S of SEQ ID NO: 49 comprises a RuvC domain and a HNH domain, nickase variants can be engineered, e.g., via disrupting the nuclease activity of either the RuvC domain or the HNH domain.

The variant CRISPR nuclease polypeptides provided herein are derived from the reference CRISPR Nuclease S of SEQ ID NO: 49, e.g., via introducing one or more mutations to the reference CRISPR nuclease to enhance one or more activities of the nuclease.

The variant CRISPR nuclease polypeptide provided herein can contain one or more alterations relative to the reference CRISPR Nuclease S of SEQ ID NO: 49, e.g., one or more amino acid residue substitutions, one or more deletions, one or more insertions, one or more fusions, or a combination thereof. In some instances, the alterations may be introduced into the BH domain, in the REC domain, in the PID domain, or a combination thereof. In some instances, no alterations are introduced into the RuvC and/or the HNH nuclease domains, or at the active sites and/or sites involved in activity in these domains as provided herein. Alternatively, conservative amino acid substitutions may be introduced into SEQ ID NO: 49, including in the RuvC and/or the HNH nuclease domains.

In some embodiments, the variant CRISPR nuclease polypeptide provided herein may comprise one or more arginine substitutions, one or more lysine substitutions, or a combination thereof relative to SEQ ID NO: 49. In some examples, the variant CRISPR nuclease polypeptide may contain up to 20 arginine and/or lysine substitutions (e.g., up to 20 arginine substitutions, up to 20 lysine substitutions, or a combination thereof), e.g., up to 19, 18, 17, 16, 15, 14, 13, 12, 11, 10, 9, 8, 7, 6, 5, 4, 3, or 2 arginine substitutions, lysine substitutions, or a combination thereof. In specific examples, the variant CRISPR nuclease polypeptide may contain 12, 11, 10, 9, 8, 7, 6, 5, 4, 3, or 2 arginine substitutions, lysine substitutions, or a combination thereof. In some examples, the variant CRISPR nuclease polypeptide provided herein contains arginine substitutions.

In some instances, the arginine and/or lysine substitutions may be located in the BH domain, in the REC domain, in the HNH nuclease domain, in the PID domain, or a combination thereof of Nuclease S. In some examples, the arginine and/or lysine substitutions may be located at the position(s) set forth in Table 12 or Table 13. For example, the arginine and/or lysine substitution may be introduced into one or more of the following positions: T72, N76, D179, Q200, E277, H280, I322, V324, V504, W509, D844, and I845 of SEQ ID NO: 49. In some examples, the one or more substitutions at one or more of these positions are arginine substitutions.

In some instances, the arginine and/or lysine substitutions may be at the following positions of SEQ ID NO: 49: (a) T72, N76, D179, Q200, E277, H280, I322, W509, D844, and I845; (b) T72, N76, D179, Q200, E277, H280, I322, V504, D844, and I845; (c) N76, D179, Q200, E277, H280, I322, W509, D844, and I845; (d) T72, D179, Q200, E277, H280, I322, W509, D844, and I845; (e) N76, D179, Q200, E277, H280, I322, V504, D844, and I845; (f) T72, D179, Q200, E277, H280, I322, V504, D844, and I845; (g) T72, D179, Q200, E277, H280, I322, V504, and D844; (h) T72, D179, Q200, E277, H280, I322, W509, and D844; (i) T72, D179, Q200, H280, I322, W509, and D844; or j) T72, D179, Q200, H280, I322, V504, and D844. In some examples, the substitutions at any of (a)-(j) are arginine substitutions.

In some instances, the one or more arginine and/or lysine substitutions may be at the following positions relative to SEQ ID NO: 49: a) W509, T72, and I845; b) H280, T72, and I845; c) W509 and I845; d) H280, W509, T72, and I845; e) D844 and W509; f) W509 and T72; g) D844, W509, and I845; h) H280, W509, and I845; or i) D844 and H280. In some examples, the substitutions at those positions are arginine substitutions.

In some specific examples, the one or more arginine and/or lysine substitutions may be at the positions of W509, T72, and I845 of SEQ ID NO: 49. In other specific examples, the one or more arginine and/or lysine substitutions may be at the positions of H280, T72, and I845 of SEQ ID NO: 49.

In some specific examples, the engineered CRISPR nuclease polypeptide derived from Nuclease S (SEQ ID NO: 49) may comprise the arginine substitutions of W509R, T72R, and I845R. In other specific examples, the engineered CRISPR nuclease polypeptide derived from Nuclease S (SEQ ID NO: 49) may comprise the arginine substitutions of H280R, T72R, and I845R.

In some instances, the arginine and/or lysine substitution may be located within the RuvC and/or the HNH nuclease domains of Nuclease S (SEQ ID NO: 49). In some examples, the arginine and/or lysine substitutions may be within the RuvC nuclease domain to reduce or inactivate the RuvC domain (e.g., at positions D25, E371, R580, H601, and/or D604 in the RuvC domain). In other examples, the arginine and/or lysine substitutions may be within the HNH domain (e.g., at positions H448, E447, and/or N471 in HNH domain). In yet other examples, the arginine and/or lysine substitutions may be within both the RuvC domain and the HNH domain to reduce or diminish the nuclease enzymatic activity.

Alternatively, the arginine and/or lysine substitution may not be at the active sites and/or sites involved in activity in the RuvC and/or the HNH nuclease domains of Nuclease S (e.g., not at positions D25, E371, R580, H601, and/or D604 in the RuvC domain and/or at positions E447, H448, and/or N471 in HNH domain of SEQ ID NO: 49). In some examples, the arginine and/or lysine substitution may not be in the RuvC and/or HNH domains. In some examples, the variant CRISPR nuclease polypeptide that contains one or more arginine and/or lysine substitutions (e.g., one or more arginine substitutions) may share a sequence identity at least 90% (e.g., 95%, 97%, 98%, 99%, 99.5%, or greater) with SEQ ID NO: 49.

Alternatively or in addition, the variant CRISPR nuclease polypeptide derived from Nuclease S provided herein may comprise one or more nickase mutations within either the RuvC or the HNH nuclease domain to reduce or eliminate the nuclease activity of the target domain, thereby producing a variant with nickase activity. Such mutations may be deletions, insertions, amino acid substitutions, or a combination thereof. In some embodiments, the mutations within either the RuvC or the HNH nuclease domain are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R. Groups of conservative amino acid residue substitutions are provided herein.

Positions D25, E371 and D604 are deemed the active sites in the RuvC domain, and positions E447, H448, and N471 are deemed the active sites in the HNH domain of Nuclease S of SEQ ID NO: 49. R580 and H601 may also be important for nuclease activity of the RuvC domain. In some examples, the one or more mutations may be within the RuvC nuclease domain to reduce or inactivate the RuvC domain (e.g., at positions D25, E371, D604, R580 and/or H601 in the RuvC domain of Nuclease S of SEQ ID NO: 49). In some specific examples, the variant CRISPR nuclease polypeptide may contain substitution(s) of D25A, E371A, and/or D604A in SEQ ID NO: 49. In other examples, any of D25, E371, and D604 may be substituted by an amino acid residue similar to A, for example, G, S, or L.

In some examples, the one or more mutations may be within the HNH domain of Nuclease S of SEQ ID NO: 49 (e.g., at positions E447, H448, and/or N471 in the HNH domain). In some specific examples, the variant CRISPR nuclease polypeptide may contain substitutions of E447A, H448A, and/or N471A in SEQ ID NO: 49. Alternatively, any of E447, H448, and N471 may be replaced with an amino acid residue similar to A, for example, G, L, or S. In one specific example, the variant CRISPR nuclease polypeptide may contain substitution of H448A. Exemplary nickase variants of Nuclease S are provided in Table 19, each of which is within the scope of the present disclosure.

In yet other examples, the one or more mutations may be within both the RuvC domain and the HNH domain of Nuclease S to reduce or diminish the nuclease enzymatic activity.

In some embodiments, the variant CRISPR nuclease polypeptide derived from Nuclease S provided herein may comprise both arginine/lysine substitutions, e.g., at one or more positions provided herein, and nickase mutations. In some instances, the variant CRISPR nuclease polypeptide may comprise arginine/lysine substitutions (e.g., arginine substitutions) listed in Table 12 or Table 13. Such variant CRISPR nuclease polypeptide may further comprise one or more mutations that reduce or inactivate the RuvC and/or HNH nuclease domain (e.g., one or more mutations within the RuvC and/or HNH nuclease domain). For example, the variant CRISPR nuclease polypeptide may comprise arginine/lysine substitutions (e.g., arginine substitutions) at positions H280, T72, and I845 or at positions W509R, T72R, and I845R, and an amino acid substitution at H448 (e.g., H448A) in SEQ ID NO: 49.

### (C) Nuclease H and Engineered Variants Thereof

In some embodiments, the CRISPR nuclease polypeptide is derived from Nuclease H, including both the wild-type Nuclease H (SEQ ID NO: 64), a variant thereof such as those disclosed herein, or a fusion polypeptide comprising such. The variant CRISPR nuclease polypeptides of Nuclease H may be produced via introducing one or more mutations to the reference CRISPR nuclease to modulate (e.g., enhance or reduce) one or more activities of the nuclease.

The reference CRISPR nuclease of SEQ ID NO: 64 (Nuclease H; see Table 14 below) is a CRISPR nuclease that comprises both a RuvC nuclease domain (located at residues 1-37, 325-357, and 522-651 of SEQ ID NO: 64) and a HNH domain (located at residues 358-521 of SEQ ID NO: 64). The RuvC nuclease domain and the HNH nuclease domain coordinate cleavage of the DNA strand adjacent to the 5′-NRC-3′, 5′-NRM-3′, 5′-NRCY-3′, or 5′-NRMY-3′ PAM motif, in which N represents any nucleotide, R represents A or G, M represents A or C, and Y represents C or T.

Positions D7, E356, and D580 are deemed the active sites in the RuvC domain, and positions E419, H420, and N443 are deemed the active sites in the HNH domain of Nuclease H. R329 and H338 may also be important for the nuclease activity of the RuvC domain. In addition to the nuclease domains, the reference CRISPR Nuclease H of SEQ ID NO: 64 also includes a BH domain (residues 38-76 of SEQ ID NO: 64), a REC domain (residues 77-324 of SEQ ID NO: 64), a PLL domain (residues 652-665), a WED domain (residues 666-807), and a PID domain (residues 808-949).

Compared to the CRISPR-Cas9 nuclease from Streptococcus pyogenes, the reference CRISPR Nuclease H of SEQ ID NO: 64 disclosed herein is smaller. The scaffold utilized by the reference CRISPR nuclease of SEQ ID NO: 64 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller CRISPR nuclease size. These features would be beneficial for delivery. Arginine substitutions can be introduced into the reference CRISPR Nuclease H of SEQ ID NO: 64 to increase indel activity.

Nuclease H recognizes different PAM motifs in genomic targets as compared with SpCas9, allowing for additional gene targets to be edited. Further, the RuvC domain of Nuclease H cleaves the non-target strand within 3 to 8 nucleotides upstream of the PAM, and the HNH cleaves the target strand between 3 and 4 nucleotides upstream of the PAM, each leading to a cut site with an overhang of 0-5 nucleotides (most commonly a 1-nucleotide overhang). This cutting pattern is different from that of SpCas9 described above.

Additionally, use of gene editing systems comprising Nuclease H or variants thereof can result in the introduction of indels into a target nucleic acid that are larger than those capable of being introduced by SpCas9. For example, insertions induced by use of Nuclease H or variants thereof can range from about 1-nucleotide to about 5-nucleotides (most commonly about 1-nucleotide or 4-nucleotides). Deletions induced by use of Nuclease H or variants thereof can range from about 1-nucleotide to about 25-nucleotides or larger (most commonly about 13-nucleotides). Further, since the reference CRISPR Nuclease H of SEQ ID NO: 64 comprises a RuvC domain and an HNH domain, nickase variants can be engineered, e.g., via disrupting the nuclease activity of either the RuvC domain or the HNH domain.

The variant CRISPR nuclease polypeptides provided herein are derived from the reference CRISPR Nuclease H of SEQ ID NO: 64 (Nuclease H), e.g., via introducing one or more mutations to the reference CRISPR nuclease to modulate (e.g., enhance or reduce) one or more activities of the nuclease.

The variant CRISPR nuclease polypeptide provided herein can contain one or more mutations relative to the reference CRISPR Nuclease H of SEQ ID NO: 64, e.g., one or more amino acid residue substitutions, one or more deletions, one or more insertions, one or more fusions, or a combination thereof. In some instances, the alterations may be introduced into the BH domain, the REC domain, the PLL domain, the WED domain, the PID domain, or a combination thereof. In some instances, no alterations are introduced into the RuvC and/or the HNH nuclease domains, or at the active sites and/or sites involved in activity of these domains as provided herein. Alternatively, conservative amino acid substitutions may be introduced into SEQ ID NO: 64, including in the RuvC and/or the HNH nuclease domains.

In some embodiments, the variant CRISPR nuclease polypeptide provided herein may comprise one or more arginine and/or lysine substitutions relative to SEQ ID NO: 64. In some examples, the variant CRISPR nuclease polypeptide may contain up to 20 arginine/lysine substitutions (e.g., arginine substitutions), e.g., up to 19, 18, 17, 16, 15, 14, 13, 12, 11, 10, 9, 8, 7, 6, 5, 4, 3, or 2 arginine/lysine substitutions (e.g., arginine substitutions). In specific examples, the variant CRISPR nuclease polypeptide may contain 12, 11, 10, 9, 8, 7, 6, 5, 4, 3, or 2 arginine/lysine substitutions (e.g., arginine substitutions).

In some instances, the arginine and/or lysine substitutions (e.g., arginine substitutions) may be located in the BH domain, in the REC domain, in the PLL domain, in the WED domain, in the PID domain, or in any of the combination thereof of Nuclease H. Alternatively or in addition, the arginine/lysine substitutions (e.g., arginine substitutions) may be located within the RuvC and/or the HNH nuclease domains. In some examples, the arginine/lysine substitutions (e.g., arginine substitutions) may be within the RuvC nuclease domain to reduce or inactivate the RuvC domain (e.g., at positions D7, E356, R556, H577 and/or D580 in the RuvC domain of SEQ ID NO: 64). In other examples, the arginine/lysine substitutions (e.g., arginine substitutions) may be within the HNH domain, e.g., at positions E419, H420, and/or N443 in HNH domain of SEQ ID NO: 64. In yet other examples, the arginine/lysine substitutions (e.g., arginine substitutions) may be within both the RuvC domain and the HNH domain to reduce or diminish the nuclease enzymatic activity.

Alternatively, the arginine/lysine substitutions (e.g., arginine substitutions) may not be at the active sites and/or sites involved in the activity of the RuvC and/or the HNH nuclease domains of Nuclease S (e.g., not at positions D7, E356, R556, H577 and/or D580 in the RuvC domain and/or at positions E419, H420, and/or N443 in HNH domain of SEQ ID NO: 64). In some examples, the arginine/lysine substitutions (e.g., arginine substitutions) may not be in the RuvC and/or HNH domains of Nuclease H.

Alternatively or in addition, the arginine/lysine substitutions (e.g., arginine substitutions) may be located at positions listed in Table 17 below. For example, the substitutions may be located at one or more positions E43, L54, A170, L323, S680, T783, and D912 of SEQ ID NO: 64. In some instances, the engineered CRISPR nuclease polypeptide derived from Nuclease H may comprise arginine/lysine substitutions at two or more of positions, for example, those listed in Table 18 below. Examples include E43, L54, A170, L323, S680, T783, and D912 of SEQ ID NO: 64 (e.g., at two of these positions or three of these positions). In specific examples, the engineered CRISPR nuclease polypeptide may comprise arginine/lysine substitutions at positions E43/A170/T783 or at positions L54/L323/T783 of SEQ ID NO: 64. In some examples, the substitutions at those positions are arginine substitutions.

Alternatively or in addition, the variant CRISPR nuclease polypeptide of Nuclease H provided herein may comprise one or more mutations (i.e., nickase mutations) within either the RuvC or the HNH nuclease domain to reduce or eliminate the nuclease activity of the target domain, thereby producing a variant with nickase activity. Such mutations may be deletions, insertions, amino acid substitutions, or a combination thereof. In some embodiments, the mutations within either the RuvC or the HNH nuclease domain are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R.

Groups of conservative amino acid residue substitutions are provided herein.

In some examples, the one or more mutations may be within the RuvC nuclease domain to reduce or inactivate the RuvC domain of Nuclease H (e.g., at positions D7, E356, R329, H338, and/or D580 in the RuvC domain of SEQ ID NO: 64). Alternatively, the one or more nickase mutations may be within the HNH domain of Nuclease H (e.g., at positions E419, H420, and/or N443 in the HNH domain of SEQ ID NO: 64). In specific examples, the variant CRISPR nuclease polypeptide derived from Nuclease H may comprise one or more nickase mutations are at one or more of positions D7, R329, H338, E356, E419, H420, N443, and/or D580 in SEQ ID NO: 64. In some instances, one or more of the original residues in either the RuvC domain or the HNH domain can be replaced with Alanine (A). Alternatively, one or more of the original residues in either the RuvC domain or the HNH domain may be substituted by an amino acid residue similar to A, for example, G, S, or L.

In yet other examples, the one or more mutations may be within both the RuvC domain and the HNH domain of Nuclease H to reduce or diminish the nuclease enzymatic activity.

In some embodiments, the variant CRISPR nuclease polypeptide derived from Nuclease H provided herein may comprise both arginine/lysine substitutions, e.g., at one or more positions provided herein, and nickase mutations, e.g., at one or more of the positions also provided herein.

In some examples, the variant CRISPR nuclease polypeptide that contains one or more arginine/lysine substitutions (e.g., arginine substitutions) may share a sequence identity at least 90% (e.g., 95%, 97%, 98%, 99%, 99.5%, or greater) with SEQ ID NO: 64.

Any of the CRISPR nuclease polypeptides provided herein, including Nuclease W (SEQ ID NO: 1), Nuclease S (SEQ ID NO: 49), or Nuclease H (SEQ ID NO: 64), and their variants provided herein may be in a fusion polypeptide format. Such a fusion polypeptide may comprise a reference CRISPR nuclease or a variant thereof and one or more additional functional moieties. As used herein, the terms “fusion” and “fused” refer to the joining of at least two nucleotide or protein molecules. For example, “fusion” and “fused” can refer to the joining of at least two polypeptide domains that are encoded by separate genes in nature. The fusion can be an N-terminal fusion, a C-terminal fusion, or an intramolecular fusion. In some aspects, the domains are transcribed and translated to produce a single polypeptide.

In some instances, the CRISPR nuclease portion in the fusion polypeptide may be the reference CRISPR nuclease of SEQ ID NO: 1 (Nuclease W), SEQ ID NO: 49 (Nuclease S), or SEQ ID NO: 64 (Nuclease H). Alternatively, the CRISPR nuclease portion in the fusion polypeptide may be a variant of any of the reference CRISPR nucleases as disclosed herein.

Exemplary additional functional moieties to include in the fusion polypeptide include a peptide tag, a fluorescent protein, a base-editing domain, a DNA methylation domain, a histone residue modification domain, a localization factor, a transcription modification factor, a light-gated control factor, a chemically inducible factor, a chromatin visualization factor, or a combination thereof.

In some embodiments, the additional functional moiety may comprise a nuclear localization signal (NLS), a nuclear export signal (NES), or a combination thereof. In some examples, the fusion polypeptide may comprise an NLS, which may be located at either the N-terminus or the C-terminus. In specific examples, the fusion polypeptide may comprise a first NLS located at the N-terminus and a second NLS located at the C-terminus. The first and second NLS fragments may be identical. Alternatively, the two NLS fragments may be different. In some embodiments, the fusion polypeptide may comprise an NLS near the N-terminus and/or near the C-terminus (e.g., within about 1, 2, 3, 4, or 5 of the first amino acid or last amino acid of the CRISPR nuclease). In some embodiments, the fusion polypeptide may comprise an NLS within a flexible loop of the CRISPR nuclease. Exemplary fusion CRISPR nuclease polypeptide comprising one or more NLS signals are provided in Tables 1, 9, and 14.

In some embodiments, the additional functional moiety may be a flexible peptide linker, for example, an XTEN peptide linker, or a G/S rich peptide linker. Examples of such peptide linkers are provided in Example 1 below, which may be applicable to any of the CRISPR nuclease polypeptides disclosed herein.

### D. Preparation of CRISPR Nuclease Polypeptides

The CRISPR nuclease polypeptides as disclosed herein may be prepared by conventional methods or the methods disclosed herein. For example, the CRISPR nuclease polypeptides can be prepared by culturing host cells such as bacteria cells or mammalian cells, capable of producing the nuclease polypeptides, isolating the nuclease polypeptides thus produced, and optionally, purifying the nuclease polypeptides. The CRISPR nuclease polypeptides thus prepared may be complexed with a gRNA.

The CRISPR nuclease polypeptides can be also prepared by an in vitro coupled transcription-translation system and optionally complexes with gRNA. Bacteria that can be used for preparation of the CRISPR nuclease polypeptides are not particularly limited as long as they can produce the CRISPR nuclease polypeptides. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### (i) Vectors

The present disclosure provides vectors for expressing the CRISPR nuclease polypeptides. In some embodiments, a vector disclosed herein includes a nucleotide sequence encoding a CRISPR nuclease polypeptide as provided herein. In some embodiments, the vector comprises a Pol II promoter or a Pol III promoter.

Expression of natural or synthetic polynucleotides is typically achieved by operably linking a polynucleotide encoding the CRISPR nuclease polypeptides to a promoter and incorporating the construct into an expression vector. The expression vector is not particularly limited as long as it includes a polynucleotide encoding the CRISPR nuclease polypeptides and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method for recombinant expression vectors is not particularly limited, and examples thereof include methods using a plasmid, a phage or a cosmid.

### (ii) Methods of Expression

The present disclosure includes a method for protein expression, comprising translating the CRISPR nuclease polypeptides described herein.

In some embodiments, a host cell described herein is used to express the CRISPR nuclease polypeptides. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production CRISPR nuclease polypeptides. After expression, the host cells can be collected and CRISPR nuclease polypeptides purified from the cultures etc. according to conventional methods (for example, filtration, centrifugation, cell disruption, gel filtration chromatography, ion exchange chromatography, etc.).

A variety of methods can be used to determine the level of production of a mature CRISPR nuclease polypeptide in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the CRISPR nuclease polypeptides (and optionally the gRNA in the gene editing system disclosed herein). Such a method may comprise providing a polyribonucleotide encoding the CRISPR nuclease polypeptide to a host cell in a subject (e.g., a human subject) wherein the polyribonucleotide encodes the CRISPR nuclease polypeptide expressing the CRISPR nuclease polypeptide from the cell.

### II. Gene Editing System

In some aspects, the present disclosure provides gene editing systems with enhanced gene editing efficiencies. The gene editing system comprises a CRISPR nuclease of SEQ ID NO: 1 (Nuclease W), SEQ ID NO: 49 (Nuclease S), or SEQ ID NO: 64 (Nuclease H), or any of the variant CRISPR nuclease polypeptides disclosed herein or a nucleic encoding the CRISPR nuclease and one or more guide RNAs (gRNAs) or nucleic acid(s) encoding the gRNAs.

### CRISPR Nuclease

In some embodiments, the gene editing system disclosed herein comprises a CRISPR nuclease polypeptide as provided herein, e.g., a variant CRISPR nuclease polypeptide comprising one or more arginine or lysine substitutions, one or more mutations in one of the nuclease domains such as in the HNH nuclease domain, additional mutations such as N-terminal truncation, or a combination thereof. See above disclosures. Such a protein component may form a complex with the gRNA(s) in the same gene editing system.

Alternatively, the gene editing system comprises a nucleic acid encoding the CRISPR nuclease polypeptide. In some instances, the nucleic acid can be an expression vector (e.g., a viral vector) for producing the encoded nuclease polypeptide in host cells. In some instances, the expression vector may further comprise a coding sequence for producing one or more gRNAs of the gene editing system. In other examples, the nucleic acid encoding the CRISPR nuclease polypeptide may be a messenger RNA (mRNA) molecule. In some instances, the mRNA molecule may further comprise a coding sequence for the gRNA of the gene editing system.

### Guide RNAs

The gene editing system disclosed herein further comprises one or more gRNAs or nuclei acid(s) encoding such. As used herein, the terms “RNA guide”, “RNA guide sequence,” or “guide RNA (gRNA)” refer to an RNA molecule or a modified RNA molecule that facilitates the targeting of a CRISPR nuclease described herein to a genomic site of interest. For example, an RNA guide can be a molecule that comprises a spacer sequence and a scaffold sequence. The spacer sequence recognizes (e.g., binds to) a site in a non-PAM strand that is complementary to a target sequence in the PAM strand, e.g., designed to be complementary to a specific nucleic acid sequence. The scaffold sequence contains a nuclease binding sequence for binding to the CRISPR nuclease. In some embodiments, the scaffold is an RNA sequence.

In some instances, the gRNA disclosed herein may further comprise a linker sequence, a 5′ end and/or 3′ end protection fragment, or a combination thereof.

### (i) Spacer Sequences

As used herein, the term “spacer” and “spacer sequence” (a.k.a., a DNA-binding sequence) is a portion in an RNA guide that is the RNA equivalent of the target sequence (a DNA sequence). The spacer contains a sequence capable of binding to the non-PAM strand via base-pairing at the site complementary to the target sequence (which is in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

The gene editing system disclosed herein comprises one or more gRNAs, each comprising a spacer for targeting a genomic site of interest and a scaffold, which is recognizable by the CRISPR nuclease polypeptide contained in the gene editing system.

In association with Nuclease W (SEQ ID NO: 1) or an engineered variant thereof as disclosed herein, the target sequence can be adjacent to a protospacer adjacent motif (PAM) of 5′-NRV-3′ or 5′-NAV-3′, in which N represents any nucleotide, R represents A or G, and V represents A, C, or G. In some examples, the PAM is 5′-NRR′3′, for example, 5′-NRG-3′ (e.g., 5′-CAG-3′). In some instances, the PAM motif is 5′-NGG-3′ in which N represents any nucleotide. In specific examples, the PAM is 5′-RRR-3′, for example, 5′-AAA-3′ or 5′-GAG-3′.

In association with Nuclease S (SEQ ID NO: 49) or an engineered variant thereof as disclosed herein, the target sequence can be adjacent to a protospacer adjacent motif (PAM) of 5′-NRCC-3′, 5′-NRNC-3′, 5′NRYM-3′, or 5′-ARCC-3′, in which N represents any nucleotide, R represents A or G, Y represents C or T, and M represents A or C.

In association with Nuclease H (SEQ ID NO: 64) or an engineered variant thereof as disclosed herein, the target sequence can be adjacent to a protospacer adjacent motif (PAM) of 5′-NRC-3′, 5′-NRM-3′, 5′-NRCY-3′, or 5′-NRMY-3′, in which N represents any nucleotide, R represents A or G, M represents A or C, and Y represents C or T.

The PAM motif is located on the 3′ end of the target sequence. As used herein, the term “protospacer adjacent motif” or “PAM sequence” refers to a DNA sequence adjacent to a target sequence. In some embodiments, a PAM sequence is required for binding of the CRISPR nuclease and/or indel activity. In a double-stranded DNA molecule, the strand containing the PAM motif is called the “PAM-strand” and the complementary strand is called the “non-PAM strand.” The gRNA binds to a site in the non-PAM strand that is complementary to a target sequence disclosed herein, and the PAM sequence as described herein is present in the PAM-strand. The PAM motif can be located upstream to the target sequence.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

In specific examples, the target sequence (corresponding to the spacer sequence) is immediately adjacent to the PAM motif. In other specific examples, the target sequence and the PAM motif may have a small gap of less than 5 (e.g., 1, 2, 3, 4, or 5) nucleotides.

A spacer sequence as disclosed herein may have a length of from about 15 nucleotides to about 30 nucleotides. For example, the spacer can have a length of from about 15 nucleotides to about 20 nucleotides, from about 15 nucleotides to about 25 nucleotides, from about 20 nucleotides to about 25 nucleotides, or from about 20 nucleotides to about 30 nucleotides. In some embodiments, the spacer in the gRNA may be generally designed to have a length of between 15 and 25 nucleotides (e.g., 15, 16, 17, 18, 19, 20, 21, 22, 23, 24, and 25) and be complementary to a specific target sequence. In some embodiments, the spacer sequence may be designed to have a length of between 18-22 nucleotides (e.g., 20 nucleotides).

In some embodiments, the spacer sequence may have at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a target sequence as described herein and is capable of binding to the complementary region of the target sequence via base-pairing.

In some embodiments, the spacer sequence comprises only RNA bases. In some embodiments, the spacer sequence comprises a DNA base (e.g., the spacer comprises at least one thymine). In some embodiments, the spacer sequence comprises RNA bases and DNA bases (e.g., the DNA-binding sequence comprises at least one thymine and at least one uracil).

### (ii) Scaffold Sequence

The scaffold sequence in the gRNA is recognizable by the CRISPR nuclease polypeptide also in the gene editing system.

In some instances, the scaffold sequence is recognizable by Nuclease W (SEQ ID NO: 1) or any of the engineered variants thereof provided herein. Such a scaffold sequence may comprise SEQ ID NO: 2.

(SEQ ID NO: 2)
GUUUCAGCUACCCUGAGUUUCUCAACUCCGUUGAGACUUCCUCAGGUU
CAAGCUGAAAAGAGCAUCUGUCCGGAAGGACCACUCCGGGUUAGGGCA
GACCCGGCUCUUGGGCUUCCUUGGUCUUUGACUCUGGAGGAAACCUUC
CGGCGUGUCGCAAGACAAGAUGUGAGCCUG

In other instances, the scaffold sequence recognizable by Nuclease W (SEQ ID NO: 1) or any of the engineered variants may be a variant derived from SEQ ID NO: 2. Such a variant scaffold sequence may comprise a nucleotide sequence at least 80% (e.g., at least 85%, 90%, 95%, 98%, or greater) identical to SEQ ID NO: 2. Alternatively or in addition, the variant scaffold sequence recognizable by Nuclease W (SEQ ID NO: 1) or any of the engineered variants may comprise deletions, nucleotide substitutions, or a combination thereof. The variant CRISPR nuclease polypeptide may have increased binding to the variant scaffold sequence as compared with the scaffold of SEQ ID NO: 2. In some examples, the variant scaffold recognizable by Nuclease W (SEQ ID NO: 1) or any of the engineered variants may be a fragment of SEQ ID NO: 2 or a variant thereof as disclosed herein. For example, the variant scaffold for use in the gRNAs provided herein may have a length ranging from 100-150.

In a gRNA, the scaffold may be located at the 3′ end of the spacer. In some instances, the scaffold and spacer are connected directly. In other instances, the scaffold and spacer may be connected via a nucleotide linker.

In some instances, the scaffold sequence is recognizable by Nuclease S (SEQ ID NO: 49) or any of the engineered variants thereof as provided herein. Such a scaffold sequence may com rise SE ID NO: 63 provided below:

(SEQ ID NO: 63)
GUUGUAUUCGGAUGGGAGUAAAAGGUUGAGUACACCUGGAAACAAAAC
GACAUAAAGGUGUUCUCCCAUCUGUGUACAAAGAGCAGAUAUAUGAGU
UGAUAAGUAAUAUGUUGCACGUAUGGACUUUGACAGUCUGUACCUCUG
CGUGAAAGACAUAUUGCGUUAUUCAUAUAUCUGUGAGUCUGUUCUAAA
UUUAAAGGUUUAGGGAA

In other instances, the scaffold sequence recognizable by Nuclease S (SEQ ID NO: 49) or any of the engineered variants thereof may be a variant derived from SEQ ID NO: 63. Such a variant scaffold sequence may comprise a nucleotide sequence at least 80% (e.g., at least 85%, 90%, 95%, 98%, or greater) identical to SEQ ID NO: 63. Alternatively or in addition, the variant scaffold sequence recognizable by Nuclease S (SEQ ID NO: 49) or any of the engineered variants thereof may comprise deletions, nucleotide substitutions, or a combination thereof. The variant CRISPR nuclease may have increased binding to a variant scaffold sequence as compared with the scaffold of SEQ ID NO: 63. In some examples, the variant scaffold recognizable by Nuclease S (SEQ ID NO: 49) or any of the engineered variants thereof may be a fragment of SEQ ID NO: 63 or a variant thereof as disclosed herein. For example, the variant scaffold for use in the gRNAs provided herein may have a length ranging from 100-150.

In a gRNA, the scaffold may be located at the 3′ end of the spacer. In some instances, the scaffold and spacer are connected directly. In other instances, the scaffold and spacer may be connected via a nucleotide linker.

In some instances, the scaffold sequence is recognizable by Nuclease H (SEQ ID NO: 64) or any of the engineered variants thereof provided herein. Such a scaffold sequence may comprise SEQ ID NO: 77 provided below:

(SEQ ID NO: 77)
GUUUCAAUCAACCUGAUCUCUCUGCUUGCGCUGCAGGCUUCAUAAGAU
CAGUAGGUUGAAAAGAGCAUCCGUCCGACAUGCACUCCGGGAUGGGGC
AGUCCCGGCUCUUGCGGUUCCCGAUGCAGUUAUGUGUCCGCGGAACAC
GGUGUCGGACACGUCGCAAGACAGGAUGUGAGCCCAUUAUCCACAUUA
GGAUCAAUGUACGCAUGAGCAGUCCGCAAUGCCGGGGACGCU

In other instances, the scaffold sequence recognizable by Nuclease H (SEQ ID NO: 64) or any of the engineered variants thereof may be a variant derived from SEQ ID NO: 77. Such a variant scaffold sequence may comprise a nucleotide sequence at least 80% (e.g., at least 85%, 90%, 95%, 98%, or greater) identical to SEQ ID NO: 77. Alternatively or in addition, the variant scaffold sequence recognizable by Nuclease H (SEQ ID NO: 64) or any of the engineered variants thereof may comprise deletions, nucleotide substitutions, or a combination thereof. The variant CRISPR nuclease polypeptide may have increased binding to the variant scaffold sequence as compared with the scaffold of SEQ ID NO: 77. In some examples, the variant scaffold recognizable by Nuclease H (SEQ ID NO: 64) or any of the engineered variants thereof may be a fragment of SEQ ID NO: 77 or a variant thereof as disclosed herein. For example, the variant scaffold for use in the gRNAs provided herein may have a length ranging from 100-150.

In a gRNA, the scaffold may be located at the 3′ end of the spacer. In some instances, the scaffold and spacer are connected directly. In other instances, the scaffold and spacer may be connected via a nucleotide linker.

(iii) Modification of Nucleic Acids

Any of the guide RNAs or encoding nucleic acids (e.g., mRNA encoding the CRISPR nuclease polypeptide) disclosed herein may include one or more modifications. Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The gRNA or any of the nucleic acid sequences encoding such may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). One or more atoms of a purine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. In some embodiments, the gRNA or any of the nucleic acid sequences encoding components of the composition may comprise an abasic site (i.e., a location that does not have a purine or a pyrimidine). Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosyleytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, any RNA sequence described herein (e.g., mRNA encoding the CRISPR nuclease polypeptide) may comprise an end modification (e.g., a 5′ end modification or a 3′ end modification). In some embodiments, the end modification is a chemical modification. In some embodiments, the end modification is a structural modification. See disclosures herein.

When a gene editing system disclosed herein comprises nucleic acids encoding the CRISPR nuclease, e.g., mRNA molecules, such nucleic acid molecules may contain any of the modifications disclosed herein, where applicable.

### III. Gene Editing Methods

Any of the gene editing systems can be used to genetically modify (edit) a target nucleic acid, which can be a genetic site of interest, e.g., a genetic site where genetic editing is needed, for example, to fix a genetic mutation, to introduce a protective mutation, to introduce modifications for modulating expression of a gene, etc.

The gene editing systems and compositions disclosed herein are applicable for editing and introducing edits into a variety of target sequences. In some embodiments, the target sequence is a DNA molecule, such as a DNA locus (referred to herein as a target sequence or an on-target sequence).

For gene editing systems comprising Nuclease W (SEQ ID NO: 1) or an engineered variant thereof as disclosed herein, the target sequence is adjacent to the PAM motif of is 5′-NRV-3′; (e.g., 5′-NAV-3′, 5′-NRR′3′ or 5′-RRR-3′), in which N represents any nucleotide, R represents A or G, and V represents A, C, or G. In some instances, the PAM motif is 3′ (downstream) to the target sequence.

For gene editing systems comprising Nuclease S (SEQ ID NO: 49) or an engineered variant thereof as disclosed herein, the target sequence is adjacent to the PAM motif of 5′-NRCC-3′, 5′-NRNC-3′, 5′NRYM-3′, or 5′-ARCC-3′, in which N represents any nucleotide, R represents A or G, Y represents C or T, and M represents A or C. In some instances, the PAM motif is 3′ (downstream) to the target sequence.

For gene editing systems comprising Nuclease H (SEQ ID NO: 64) or an engineered variant thereof as disclosed herein, the target sequence is adjacent to the PAM motif of 5′-NRC-3′, 5′-NRM-3′, 5′-NRCY-3′, or 5′-NRMY-3′, in which N represents any nucleotide, R represents A or G, M represents A or C, and Y represents C or T. In some instances, the PAM motif is 3′ (downstream) to the target sequence.

In some embodiments, the target nucleic acid is a genomic site in a cell. In some instances, the target nucleic acid where the genetic edit would occur can be in a protein-coding region. Alternatively, the target nucleic acid may be in a regulatory region, such as a promoter, enhancer, a 5′ or 3′ untranslated region. In other instances, the target nucleic acid can be in a non-coding gene, such as transposon, miRNA, tRNA, ribosomal RNA, ribozyme, or lincRNA.

### A. Gene Edits

Any of the gene editing systems disclosed herein may be used to edit a target gene of interest, e.g., a gene involved in a disease (e.g., a genetic disease). In some embodiments, the target gene can be one that is involved in an immune response in a subject. For example, the target gene can be an immune checkpoint gene or a tumor necrosis factor receptor superfamily member. The gene edit may occur in an exon (e.g., in a coding region). Alternatively, the gene editing may occur in an intron or in a regulatory element (e.g., promoter, enhancer, inhibitory element, etc.). In some instances, the gene edit may result in reducing or eliminating the expression of the target gene. In other instances, the gene edit may result in enhancing expression of the target gene (e.g., disrupting an inhibitory factor).

In some aspects, provided herein are methods for introducing at least one edit into a target nucleic acid (e.g., a genomic site of interest such as in any of the target genes disclosed herein) using the gene editing system described herein.

As used herein, the term “edit” refers to one or more modifications introduced into a nucleotide sequence in a target nucleic acid such as in a genomic site of interest. The edit may occur within a target sequence as defined herein. Alternatively, the edit may occur outside the target sequence (e.g., adjacent to the target sequence). The edit can be one or more substitutions, one or more insertions, one or more deletions, or a combination thereof.

Deletion refers to a loss of a nucleotide or nucleotides in a nucleic acid sequence, relative to a reference sequence. No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to an insertion of up to several kilobases.

Insertion refers to a gain of a nucleotide or nucleotides in a nucleic acid sequence, relative to a reference sequence. No particular process is implied in how to make a sequence comprising an insertion. For instance, a sequence comprising an insertion can be synthesized directly from individual nucleotides. In other embodiments, an insertion is made by providing and then altering a reference sequence. The nucleic acid sequence can be in a genome of an organism. The nucleic acid sequence can be in a cell. The nucleic acid sequence can be a DNA sequence. The insertion can be a frameshift mutation or a non-frameshift mutation. An insertion described herein refers to an insertion of up to several kilobases.

In some embodiments, the gene editing methods disclosed herein may introduce edits, including a substitution, an insertion, a deletion, or a combination thereof, into the target nucleic acid.

In some examples, the edits can include at least one substitution, at least one insertion, and/or at least one deletion. In some embodiments, the edit comprises at least one substitution, insertion, or deletion. In some embodiments, the substitution, insertion, or deletion is at least 1-500 nucleotides (e.g., 1-10 nucleotides, 10-30 nucleotides, 30-50 nucleotides, 50-100 nucleotides, 100-200 nucleotides, 200-300 nucleotides, 300-400 nucleotides, or 400-500 nucleotides).

In some examples, the edit may occur within about 500 nucleotides of a PAM sequence (e.g., a PAM sequence associated with Nuclease W, Nuclease S, or Nuclease H as disclosed herein). In some embodiments, the edit occurs adjacent to the PAM sequence, e.g., within about 1-500 nucleotides upstream or downstream of the PAM sequence. In some embodiments, the edit may occur within about 1-10 nucleotides, 10-30 nucleotides, 30-50 nucleotides, 50-100 nucleotides, 100-200 nucleotides, 200-300 nucleotides, 300-400 nucleotides, or 400-500 nucleotides upstream of the PAM sequence. Alternatively or in addition, the edit may occur within about 1-10 nucleotides, 10-30 nucleotides, 30-50 nucleotides, 50-100 nucleotides, 100-200 nucleotides, 200-300 nucleotides, 300-400 nucleotides, or 400-500 nucleotides downstream of the PAM sequence.

In some embodiments, the edit starts at the PAM sequence. In some embodiments, the edit may start within about 1-30 nucleotide downstream of the PAM. Alternatively, the edits may start within about 1-30 nucleotide upstream of the PAM.

In some embodiments, the edit may end within about 1-300 nucleotides upstream of the PAM sequence, for example, ends within about 1-10 nucleotides, about 10-30 nucleotides, about 30-50 nucleotides, about 50-100 nucleotides, about 100-200 nucleotides, or about 200-300 nucleotides upstream of the PAM sequence. Alternatively, the edit may end within about 1-300 nucleotides downstream of the PAM sequence, for example, ends within about 1-10 nucleotides, about 10-30 nucleotides, about 30-50 nucleotides, about 50-100 nucleotides, about 100-200 nucleotides, or about 200-300 nucleotides downstream of the PAM sequence.

In some embodiments, the edit may end at the PAM sequence. In some embodiments, the edit ends within about 1-30 nucleotide downstream of the PAM. In other embodiments, the edit may end within about 1-30 nucleotide upstream of the PAM.

### B. Gene Editing in Cells

In some aspects, provided herein are methods for editing a genomic site of interest (e.g., a target gene as disclosed herein) in cells using any of the gene editing systems disclosed herein. To perform this method, the gene editing system can be delivered to or introduced into a population of cells. In some instances, cells comprising the desired genetic editing may be collected and optionally cultured and expanded in vitro.

The cell described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell.

In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a primate cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, HEK293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a mesenchymal stem cell. In some embodiments, the cell is an embryonic stem cell. In some embodiments, the cell is a hematopoietic stem cell. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a glial cell. In some embodiments, the cell is a pancreatic islet cell, including an alpha cell, beta cell, delta cell, or enterochromaffin cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or primate cell or a murine cell. In some embodiments, the murine cell is derived from a wild-type mouse, an immunosuppressed mouse, or a disease-specific mouse model. In some embodiments, the cell is a cell within a living tissue, organ, or organism.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In embodiments wherein a gene editing system disclosed herein is introduced into a plurality of cells, at least about 0.5% of the cells comprise the desired edit. In some embodiments, at least about 1% of the cells comprise the desired edit. In some embodiments, at least about 2% of the cells comprise the desired edit. In some embodiments, at least about 3% of the cells comprise the desired edit. In some embodiments, at least about 4% of the cells comprise the desired edit. In some embodiments, at least about 5% of the cells comprise the desired edit. In some embodiments, at least about 10% of the cells comprise the desired edit. In some embodiments, at least about 20% of the cells comprise the desired edit. In some embodiments, at least about 30% of the cells comprise the desired edit. In some embodiments, at least about 40% of the cells comprise the desired edit. In some embodiments, at least about 50% of the cells comprise the desired edit.

The cells carrying the desired genetic edit, e.g., produced by the method disclosed herein using any of the gene editing systems also disclosed herein, are also within the scope of the present disclosure. In some instances, the cells modified by the CRISPR nuclease polypeptide disclosed herein may be useful as an expression system to manufacture biomolecules. For example, the modified cells may be useful to produce biomolecules such as proteins (e.g., cytokines, antibodies, antibody-based molecules), peptides, lipids, carbohydrates, nucleic acids, amino acids, and vitamins. In other embodiments, the modified cell may be useful in the production of a viral vector such as a lentivirus, adenovirus, adeno-associated virus, and oncolytic virus vector. In some embodiments, the modified cell may be useful in cytotoxicity studies. In some embodiments, the modified cell may be useful as a disease model. In some embodiments, the modified cell may be useful in vaccine production. In some embodiments, the modified cell may be useful in therapeutics. For example, in some embodiments, the modified cell may be useful in cellular therapies such as transfusions and transplantations.

In some embodiments, the cells modified by the CRISPR nuclease polypeptide as disclosed herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion of the modified stem cell. The disclosure further provides organisms (such as animals, plants, or fungi) comprising or produced from a modified cell of the disclosure.

### C. Delivery of Gene Editing Systems to Cells

In some embodiments, any of the gene editing systems or components thereof as disclosed herein may be formulated, for example, including a carrier, such as a carrier and/or a polymeric carrier, e.g., a liposome or lipid nanoparticle, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the CRISPR nuclease polypeptide and/or the gRNAs, and/or a pre-formed ribonucleoprotein to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells. In some embodiments, a composition of the present invention is further delivered with an agent (e.g., compound, molecule, or biomolecule) that affects DNA repair or DNA repair machinery. In some embodiments, a composition of the present invention is further delivered with an agent (e.g., compound, molecule, or biomolecule) that affects the cell cycle.

In some embodiments, a first composition comprising a CRISPR nuclease polypeptide is delivered to a cell. In some embodiments, a second composition comprising a gRNA is delivered to the cell. In some embodiments, the first composition is contacted with a cell before the second composition is contacted with the cell. In some embodiments, the first composition is contacted with a cell at the same time as the second composition is contacted with the cell. In some embodiments, the first composition is contacted with a cell after the second composition is contacted with the cell. In some embodiments, the first composition is delivered by a first delivery method and the second composition is delivered by a second delivery method. In some embodiments, the first delivery method is the same as the second delivery method. For example, in some embodiments, the first composition and the second composition are delivered via viral delivery. In some embodiments, the first delivery method is different than the second delivery method. For example, in some embodiments, the first composition is delivered by viral delivery and the second composition is delivered by lipid nanoparticle-mediated transfer and the second composition is delivered by viral delivery or the first composition is delivered by lipid nanoparticle-mediated transfer and the second composition is delivered by viral delivery.

In some examples, any of the gene editing systems provided herein comprises one or more lipid excipients, which associate with the components of the gene editing system, to facilitate delivery of the gene editing system to host cells. In some instances, the one or more lipid excipients may form lipid nanoparticles (LNPs), which associate with or encapsulating components of the gene editing system (e.g., a mRNA molecule encoding the CRISPR nuclease polypeptide and the gRNA). Such LNP-containing gene editing systems can be brought in contact with host cells (e.g., administering to a subject in need of gene editing) and the LNP can facilitate delivery of the gene editing system into the host cells.

In other examples, the gene editing system provided herein may be delivered to host cells via a viral vector-mediated approach. Such a gene editing system may comprise a viral vector that carries a transgene encoding the CRISPR nuclease polypeptide and optionally a transgene encoding the gRNA. In some instances, the viral vector may be an AAV vector. The viral vector may facilitate delivery of the transgene(s) into host cells where the transgene(s) produces the CRISPR nuclease polypeptide and optionally the gRNA to effect genetic editing of a target gene.

### IV. Therapeutic Applications

Any of the gene editing systems or modified cells generated using such a gene editing system as disclosed herein may be used for treating a disease that may be benefit from the gene edit introduced by the gene editing system or carried by the modified cells. For example, the disease may be a genetic disease and the gene edit fixes the gene mutation associated with the genetic disease. Alternatively, the disease may be associated with abnormal expression of a gene and the gene edit rescues such abnormal expression.

In some embodiments, provided herein is a method for treating a disease comprising administering to a subject (e.g., a human patient) in need of the treatment any of the gene editing system disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by the CRISPR nuclease polypeptide and the gRNA as disclosed herein. In some instances, the modified cells may be a heterogenous population comprising cells with different types of gene edits. Alternatively, the modified cells may comprise a substantially homogenous cell population (e.g., at least 80% of the cells in the whole population) comprising one particular gene edit. In some examples, the cells can be suspended in a suitable media.

In some embodiments, provided herein is a composition comprising the gene editing system or components thereof or the modified cells. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits or systems that can be used, for example, to carry out a method described herein. In some embodiments, the kits or systems include a CRISPR nuclease polypeptide and optionally a gRNA. In some embodiments, the kits or systems include a polynucleotide that encodes the CRISPR nuclease polypeptide and optionally the gRNA. The gRNA of the kits can be designed to target a sequence of interest. The CRISPR nuclease polypeptide and the gRNA can be packaged within the same vial or other vessel within a kit or system or can be packaged in separate vials or other vessels, the contents of which can be mixed prior to use. The kits or systems can additionally include, optionally, a buffer and/or instructions for use of the CRISPR nuclease polypeptide and the gRNA.

In some embodiments, the kit comprises a first composition comprising a CRISPR nuclease polypeptide as disclosed herein. In some embodiments, the kit comprises a second composition comprising a gRNA as also disclosed herein. In some embodiments, the first composition and the second composition are packaged within the same vial. In some embodiments, the first composition and the second composition are packaged within different vials.

In some embodiments, the kit may be useful for research purposes. For example, in some embodiments, the kit may be useful to study gene function.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to further illustrate some embodiments of the present disclosure but are not intended to limit the scope of the present disclosure; it will be understood by their exemplary nature that other procedures, methodologies, or techniques known to those skilled in the art may alternatively be used.

### Example 1: CRISPR Nuclease Mediated Editing of Human Target Sequences in HEK293T Cells

This Example describes the genomic editing of the AAVS1, EMX1, and VEGFA genes by the CRISPR Nuclease W (SEQ ID NO: 1) introduced into cells by lipid-based transient transfection into the HEK293T cell line.

Nuclease W was tagged with an N-terminal SV40 nuclear localization sequence (NLS) and a C-terminal XTEN linker directly upstream of a nucleoplasmin NLS, and its coding sequence was converted to a human codon-optimized DNA sequence, synthesized, and cloned into a pcDNA3.1 vector (Invitrogen), containing a CMV promoter for expression. The reference and NLS-tagged sequences used are in Table 1. Plasmids were purified using a midiprep kit.

TABLE 1
Amino Acid Sequences

| | Nucleotide sequence | |
|---|---|---|
| Component | (codon optimized for human cells) | Amino acid sequence |
| Reference | atgagctcaacacacacactggccatcgatttcggcagcaagaac | MSSTHTLAIDFGSKNIGIALV |
| Nuclease W | atcggcatcgccctggtgcggaatcaggacagcctgaacacccct | RNQDSLNTPLFAGTALYDRF |
| | ctgttcgctggcacagccctgtacgacagattcacactatccaagaa | TLSKKVEPRVQMRRMRRTK |
| | ggtggaacctcgggtgcagatgcgacggatgagaagaaccaaga | KTRKARLAHLEAGLLSHGLE |
| | agacaagaaaggccaggctggcccacctggaggccggcctgctc | PDVVVWLVNFCRRRGFKSL |
| | tctcacggcctggagcctgacgttgtggtgtggctggtgaacttctg | FDEGREPRGEKKEEEAEVFR |
| | cagaagaagaggcttcaaaagcctgtttgacgagggccgggaac | FSREEFFSALENELLERLPEE |
| | cccggggcgaaaagaaggaggaagaggctgaggtttttcggttta | KLIPVLDLCEHGLNRDGDRF |
| | gcagagaagaattcttctccgccctggagaacgagctgctggaaa | KEVRLIRIDNRGASRCAWDG |
| | gactaccagaggaaaagctgatccctgtgctggacctgtgcgagc | CNSVTPRRDNALRDALAQF |
| | atggcctgaatagagatggcgaccgtttcaaggaggtgcgtcttatc | VFTVYAQKVRSDPDLYEKIQ |
| | agaatcgacaaccggggcgctagcagatgcgcctgggacggctg | RMLDQLAQLGKRLRHVGGS |
| | taacagcgtgacccctagaagagataatgccctgagagacgccctt | DPKKERQVLLKRIREELKLL |
| | gcccagttcgtgttcaccgtgtacgcccagaaggtgagatctgatc | KFLSGLAQDLPEETIEPKEA |
| | ccgacctgtacgagaaaatccagaggatgctggaccagctagccc | WTHIRRNLMNLIEQSGGRNR |
| | agctgggcaagagactgagacatgtgggcggatctgatcctaaga | FCRHHSAQYVYHLIEGKPIPF |
| | aggaacgccaggtgctgctgaagcggatccgggaagagctgaaa | KRSLTESDMTSRREEILFQKL |
| | ctcctgaagttcctgagcggcctggcccaagatctgccagaggag | WRYIEARVLPLAPAGIDRVV |
| | acaatcgagcctaaagaggcctggacccacatcagaagaaacctg | VERTAFDMLAGSRKQRQKV |
| | atgaacctgatcgagcagtccggcggcagaaacagattttgcaga | ADDYLEEMYQQGPRYGFKD |
| | caccacagcgctcaatacgtgtaccacctcatcgaaggcaagccta | DLEMLRKEFNGLCAYCGKP |
| | tccccttcaagagatccctgaccgagagcgacatgaccagcagac | SETIVEREHILPKANFFFDSY |
| | gggaagaaatcctgttccagaaactgtggcgatatatcgaggccag | LNMVPACPDCNRMWKAKA |
| | agtgctgccactggcccctgccggaattgacagggtggtcgtgga | SPGASGLRINESAYQAYSDY |
| | aagaacagccttcgacatgctggccggatctcggaaacagaggca | LADKFKTRPPHLFHTIKKGIL |
| | gaaagtggccgacgactacctggaagaaatgtaccagcagggcc | KLMTQKDRVLEAERLLGMI |
| | cccggtacggcttcaaggatgacctggagatgctgagaaaggaat | AENLGRVVESQRGPRPLARY |
| | ttaacggcctttgcgcctactgcggtaaacctagcgaaaccatcgtg | LCEKLRQRYDRVTQIAFRSG |
| | gagagagagcacatcctgccgaaggccaacttcttcttcgacagct | RHTAIWRQAAYPDFDKAKE |
| | acctgaacatggtgcccgcgtgtcctgattgtaacagaatgtggaa | KVSGGTINHALDAMILACDL |
| | ggccaaggccagccctggagcctctgggctgcggattaacgagtc | PDVTALEARNLPPWIISAWV |
| | tgcttaccaggcttacagcgattacctcgctgacaaattcaagacca | DKVRSAAPQAGQEGIPAIPH |
| | gaccaccacacctgttccacaccatcaagaaaggcatcctgaagct | PDFAVPEFEKVLQGNFIETDL |
| | gatgacacaaaaggacaggglgctggaggccgagagactgctgg | AKLNWNRKHSRVQRQDAY |
| | gcatgatcgccgaaaacctgggaagagtggtggaaagccagcgg | GWCGLEDVPAKRIAAANLA |
| | ggacctagacctttggctcgctacctgtgcgagaaactgagacagc | ASLRNADKEKTPEDRQKEV |
| | ggtacgaccgggtgacccagatcgcctttagaagcggacggcac | KKEVVVVVHPRLRAVLEAA |
| | accgccatctggagacaggctgcttatcctgatttcgacaaggcca | NTGERPGESSAAALISWLRK |
| | aggaaaaggtcagcggcggcaccatcaatcacgccctcgacgct | AVKGSLKDARLSSHPADQA |
| | atgatcctggcctgtgatctgcccgacgtgaccgccctggaggcca | RAKALRDFADGTGEAIPAVI |
| | gaaacctgcctccttggatcatcagcgcgtgggtggacaaggtga | GVRIRYPWLKANMDLHRVE |
| | gaagcgccgcacctcaggccggccaggagggaatcccagctatc | RQTGQIVHRYVADPANQALI |
| | cctcatcctgatttcgccgtgcccgagttcgagaaggtcctgcagg | VAYLGSNGKVRRDKPMTLE |
| | gcaactttatcgagacagacctggctaagctgaactggaacagaaa | LRQSGAVVPGFKALSEVPAG |
| | gcactctagagtgcagcgacaggacgcctatggctggtgcggcct | PLQGRALGQPRPDPGEWQA |
| | ggaggacgtgcccgccaagagaatcgccgctgcaaatctggctg | ALHAYLQSAEVAEYAMVSQ |
| | ctagtctgagaaacgccgacaaggagaagacccccgaggacag | GCVVHYEDGSERYIRNFSSS |
| | acagaaagaggtgaagaaggaggtggtggtggtggttcaccctag | YGFKKSLFKGMVGVRRSPLS |
| | actgcgggctgttctggaggccgccaataccggcgaaagacctgg | GRVIPNIRI (SEQ ID NO: 1) |
| | tgaaagctccgccgccgccctgattagctggctgagaaaggccgt | |
| | gaagggaagcctgaaagatgctcgcctcagcagccaccccgccg | |
| | accaggccagagctaaagccctgagagacttcgccgacggcacg | |
| | ggcgaggccatccctgccgtgatcggcgtgagaatccggtacccc | |
| | tggctgaaggccaacatggacctgcaccgggtggaacggcagac | |
| | aggccagatcgtgcacagatatgtggccgaccccgctaaccaggc | |
| | cctgatcgttgcctacctgggcagcaacggcaaggtgagaagaga | |
| | caagcccatgaccctggagctgcgccaatctggagccgtggtgcc | |
| | tggcttcaaggcactgtctgaagtccccgccggccctctgcagggc | |
| | agagccctgggccaacctcggcctgatcctggagagtggcaggc | |
| | cgctctgcacgcctacctgcagtccgccgaagtggccgagtacgc | |
| | catggtttcccagggctgcgtggtgcactacgaggatggctctgag | |
| | cggtacataagaaatttcagcagcagctacggctttaaaaagagcc | |
| | tgttcaagggcatggtcggagtgcggcggtctccactgagcggga | |
| | gagtgatccccaacatcagaatt (SEQ ID NO: 7) | |
| SV40 NLS | aaacggacagccgacggaagcgagttcgagtcaccaaagaaga | KRTADGSEFESPKKKRKV |
| | agcggaaagtc (SEQ ID NO: 6) | (SEQ ID NO: 3) |
| Nucleo- | aaaaggccggcggccacgaaaaaggccggccaggcaaaaaag | KRPAATKKAGQAKKKK |
| plasmin | aaaaag (SEQ ID NO: 8) | (SEQ ID NO: 4) |
| NLS | | |
| XTEN | tctggaggatctagcggaggatcctctggcagcgagacaccagga | SGGSSGGSSGSETPGTSESAT |
| linker | acaagcgagtcagcaacaccagagagcagtggcggcagcagcg | PESSGGSSGGSS |
| | gcggcagcagc (SEQ ID NO: 9) | (SEQ ID NO: 5) |
| NLS-tagged | atgaaacggacagccgacggaagcgagttcgagtcaccaaagaa | MKRTADGSEFESPKKKRKVSS |
| Reference | gaagcggaaagtcagctcaacacacacactggccatcgatttcgg | THTLAIDFGSKNIGIALVRNQ |
| Nuclease W | cagcaagaacatcggcatcgccctggtgcggaatcaggacagcct | DSLNTPLFAGTALYDRFTLS |
| | gaacacccctctgttcgctggcacagccctgtacgacagattcaca | KKVEPRVQMRRMRRTKKTR |
| | ctatccaagaaggtggaacctcgggtgcagatgcgacggatgaga | KARLAHLEAGLLSHGLEPDV |
| | agaaccaagaagacaagaaaggccaggctggcccacctggagg | VVWLVNFCRRRGFKSLFDE |
| | ccggcctgctctctcacggcctggagcctgacgttgtggtgtggct | GREPRGEKKEEEAEVFRFSR |
| | ggtgaacttctgcagaagaagaggcttcaaaagcctgtttgacgag | EEFFSALENELLERLPEEKLIP |
| | ggccgggaaccccggggcgaaaagaaggaggaagaggctgag | VLDLCEHGLNRDGDRFKEV |
| | gtttttcggtttagcagagaagaattcttctccgccctggagaacgag | RLIRIDNRGASRCAWDGCNS |
| | ctgctggaaagactaccagaggaaaagctgatccctgtgctggac | VTPRRDNALRDALAQFVFTV |
| | ctgtgcgagcatggcctgaatagagatggcgaccgtttcaaggag | YAQKVRSDPDLYEKIQRML |
| | gtgcgtcttatcagaatcgacaaccggggcgctagcagatgcgcct | DQLAQLGKRLRHVGGSDPK |
| | gggacggctgtaacagcgtgacccctagaagagataatgccctga | KERQVLLKRIREELKLLKFLS |
| | gagacgcccttgcccagttcgtgttcaccgtgtacgcccagaaggt | GLAQDLPEETIEPKEAWTHIR |
| | gagatctgatcccgacctgtacgagaaaatccagaggatgctgga | RNLMNLIEQSGGRNRFCRHH |
| | ccagctagcccagctgggcaagagactgagacatgtgggggat | SAQYVYHLIEGKPIPFKRSLT |
| | ctgatcctaagaaggaacgccaggtgctgctgaagcggatccggg | ESDMTSRREEILFQKLWRYIE |
| | aagagctgaaactcctgaagttcctgagcggcctggcccaagatct | ARVLPLAPAGIDRVVVERTA |
| | gccagaggagacaatcgagcctaaagaggcctggacccacatca | FDMLAGSRKQRQKVADDYL |
| | gaagaaacctgatgaacctgatcgagcagtccggcggcagaaac | EEMYQQGPRYGFKDDLEML |
| | agattttgcagacaccacagcgctcaatacgtgtaccacctcatcga | RKEFNGLCAYCGKPSETIVE |
| | aggcaagcctatccccttcaagagatccctgaccgagagcgacat | REHILPKANFFFDSYLNMVP |
| | gaccagcagacgggaagaaatcctgttccagaaactgtggcgata | ACPDCNRMWKAKASPGASG |
| | tatcgaggccagagtgctgccactggcccctgccggaattgacag | LRINESAYQAYSDYLADKFK |
| | ggtggtcgtggaaagaacagccttcgacatgctggccggatctcg | TRPPHLFHTIKKGILKLMTQK |
| | gaaacagaggcagaaagtggccgacgactacctggaagaaatgt | DRVLEAERLLGMIAENLGRV |
| | accagcagggcccccggtacggcttcaaggatgacctggagatg | VESQRGPRPLARYLCEKLRQ |
| | ctgagaaaggaatttaacggcctttgcgcctactgcggtaaacctag | RYDRVTQIAFRSGRHTAIWR |
| | cgaaaccatcgtggagagagagcacatcctgccgaaggccaactt | QAAYPDFDKAKEKVSGGTIN |
| | cttcttcgacagctacctgaacatggtgcccgcgtgtcctgattgtaa | HALDAMILACDLPDVTALEA |
| | cagaatgtggaaggccaaggccagccctggagcctctgggctgc | RNLPPWIISAWVDKVRSAAP |
| | ggattaacgagtctgcttaccaggcttacagcgattacctcgctgac | QAGQEGIPAIPHPDFAVPEFE |
| | aaattcaagaccagaccaccacacctgttccacaccatcaagaaag | KVLQGNFIETDLAKLNWNR |
| | gcatcctgaagctgatgacacaaaaggacagggtgctggaggcc | KHSRVQRQDAYGWCGLED |
| | gagagactgctgggcatgatcgccgaaaacctgggaagagtggt | VPAKRIAAANLAASLRNAD |
| | ggaaagccagcggggacctagacctttggctcgctacctgtgcga | KEKTPEDRQKEVKKEVVVV |
| | gaaactgagacagcggtacgaccgggtgacccagatcgcctttag | VHPRLRAVLEAANTGERPGE |
| | aagcggacggcacaccgccatctggagacaggctgcttatcctga | SSAAALISWLRKAVKGSLKD |
| | ttcgacaaggccaaggaaaaggtcagcggcggcaccatcaatca | ARLSSHPADQARAKALRDF |
| | cgccctcgacgctatgatcctggcctgtgatctgcccgacgtgacc | ADGTGEAIPAVIGVRIRYPW |
| | gccctggaggccagaaacctgcctccttggatcatcagcgcgtgg | LKANMDLHRVERQTGQIVH |
| | gtggacaaggtgagaagcgccgcacctcaggccggccaggagg | RYVADPANQALIVAYLGSN |
| | gaatcccagctatccctcatcctgatttcgccgtgcccgagttcgag | GKVRRDKPMTLELRQSGAV |
| | aaggtcctgcagggcaactttatcgagacagacctggctaagctga | VPGFKALSEVPAGPLQGRAL |
| | actggaacagaaagcactctagagtgcagcgacaggacgcctatg | GQPRPDPGEWQAALHAYLQ |
| | gctggtgcggcctggaggacgtgcccgccaagagaatcgccgct | SAEVAEYAMVSQGCVVHYE |
| | gcaaatctggctgctagtctgagaaacgccgacaaggagaagacc | DGSERYIRNFSSSYGFKKSLF |
| | cccgaggacagacagaaagaggtgaagaaggaggtggtggtgg | KGMVGVRRSPLSGRVIPNIRI |
| | tggttcaccctagactgcgggctgttctggaggccgccaataccgg | SGGSSGGSSGSETPGTSESAT |
| | cgaaagacctggtgaaagctccgccgccgccctgattagctggct | PESSGGSSGGSSKRPAATKKA |
| | gagaaaggccgtgaagggaagcctgaaagatgctcgcctcagca | GQAKKKK (SEQ ID NO: 10) |
| | gccaccccgccgaccaggccagagctaaagccctgagagacttc | |
| | gccgacggcacgggcgaggccatccctgccgtgatcggcgtgag | |
| | aatccggtacccctggctgaaggccaacatggacctgcaccgggt | |
| | ggaacggcagacaggccagatcgtgcacagatatgtggccgacc | |
| | ccgctaaccaggccctgatcgttgcctacctgggcagcaacggca | |
| | aggtgagaagagacaagcccatgaccctggagctgcgccaatct | |
| | ggagccgtggtgcctggcttcaaggcactgtctgaagtccccgcc | |
| | ggccctctgcagggcagagccctgggccaacctcggcctgatcct | |
| | ggagagtggcaggccgctctgcacgcctacctgcagtccgccga | |
| | agtggccgagtacgccatggtttcccagggctgcgtggtgcactac | |
| | gaggatggctctgagcggtacataagaaatttcagcagcagctacg | |
| | gctttaaaaagagcctgttcaagggcatggtcggagtgcggcggtc | |
| | tccactgagcgggagagtgatccccaacatcagaatttctggagga | |
| | tctagcggaggatcctctggcagcgagacaccaggaacaagcga | |
| | gtcagcaacaccagagagcagtggcggcagcagcggcggcagc | |
| | agcaaaaggccggcggccacgaaaaaggccggccaggcaaaa | |
| | aagaaaaag | |
| | (SEQ ID NO: 48) | |

RNA guides were designed and cloned and into a pUC19 plasmid following the U6 PolIII promoter and terminated with a 6×polyT sequence. RNA guides were designed to be specific to target sequences within the coding exons of AAVS1, EMX1, and VEGFA with 5′-NRR-3′ PAM sequences (the PAM sequence is on the 3′ end of the target sequence). See all RNA guide sequences in Table 2. The U6 PolIII promoter uses a +1 G at the start of the transcript (i.e., the 5′ end of the RNA) for more efficient transcription that is excluded from the sequences described in Table 2. Plasmids were purified using a midiprep kit.

TABLE 2
Target and RNA Guide Sequences

| | | Target/Spacer | |
|---|---|---|---|
| Guide | PAM | Sequence | RNA Guide Sequence* |
| AVS1- | CAG | GGTGCGTCCT | GGUGCGUCCUAGGUGUUCACguuucagcuacccug |
| T3 | | AGGTGTTCAC | aguuucucaacuccguugagacuuccucagguucaagcugaaaag |
| | | (SEQ ID NO: 11) | agcaucuguccggaaggaccacuccggguuagggcagacccggcu |
| | | | cuugggcuuccuuggucuuugacucuggaggaaaccuuccggcg |
| | | | ugucgcaagacaagaugugagccug (SEQ ID NO: 12) |
| AAVS1- | AAG | TTAAAGCGAC | UUAAAGCGACUCCAAUGCGGGuuucagcuacccu |
| T5 | | TCCAATGCGG | gaguuucucaacuccguugagacuuccucagguucaagcugaaaa |
| | | (SEQ ID NO: 13) | gagcaucuguccggaaggaccacuccggguuagggcagacccggc |
| | | | ucuugggeuuccuuggucuuugacucuggaggaaaccuuccggc |
| | | | gugucgcaagacaagaugugagccug (SEQ ID NO: 14) |
| EMX1- | TAG | ATAGGCCAGA | AUAGGCCAGAGGCAGCUUCCguuucagcuacccug |
| T2 | | GGCAGCTTCC | aguuucucaacuccguugagacuuccucagguucaagcugaaaag |
| | | (SEQ ID NO: 15) | agcaucuguccggaaggaccacuccggguuagggcagacccggcu |
| | | | cuugggcuuccuuggucuuugacucuggaggaaaccuuccggcg |
| | | | ugucgcaagacaagaugugagccug (SEQ ID NO: 16) |
| EMX1- | AAA | TAAACTACAG | UAAACUACAGUGGUGCCUGGguuucagcuacccu |
| T5 | | TGGTGCCTGG | gaguuucucaacuccguugagacuuccucagguucaagcugaaaa |
| | | (SEQ ID NO: 17) | gagcaucuguccggaaggaccacuccggguuagggcagacccggc |
| | | | ucuugggcuuccuuggucuuugacucuggaggaaaccuuccggc |
| | | | gugucgcaagacaagaugugagccug (SEQ ID NO: 18) |
| VEGFA- | TAG | AGGCTCTGGA | AGGCUCUGGAGAGAUUUGUGguuucagcuacccu |
| T4 | | GAGATTTGTG | gaguuucucaacuccguugagacuuccucagguucaagcugaaaa |
| | | (SEQ ID NO: 19) | gagcaucuguccggaaggaccacuccggguuagggcagacccggc |
| | | | ucuugggcuuccuuggucuuugacucuggaggaaaccuuccggc |
| | | | gugucgcaagacaagaugugagccug (SEQ ID NO: 20) |
| VEGFA- | GAG | CCCAGTATGG | CCCAGUAUGGGCAAAGGAAAguuucagcuacccu |
| T6 | | GCAAAGGAAA | gaguuucucaacuccguugagacuuccucagguucaagcugaaaa |
| | | (SEQ ID NO: 21) | gagcaucuguccggaaggaccacuccggguuagggcagacccggc |
| | | | ucuugggcuuccuuggucuuugacucuggaggaaaccuuccggc |
| | | | gugucgcaagacaagaugugagccug (SEQ ID NO: 22) |

*Spacer in upper case and scaffold (SEQ ID NO: 2) in lower case

Approximately 16 hours prior to transfection, 25,000 HEK293T cells in DMEM/10% FBS+Pen/Strep (D10 media) were plated into each well of a 96-well plate. On the day of transfection, the cells were 50-90% confluent. For each well to be transfected, a mixture of Lipofectamine 2000™ (ThermoFisher Scientific) and Opti-MEM™ (ThermoFisher Scientific) was prepared and incubated at room temperature for 5 minutes (Solution 1). After incubation, the Lipofectamine 2000™:Opti-MEM™ mixture was added to a separate mixture containing the CRISPR nuclease plasmid (NLS-tagged), RNA guide plasmid, and Opti-MEM™ (Solution 2). In the case of negative controls, the CRISPR nuclease plasmid was excluded. Solutions 1 and 2 were mixed by pipetting up and down, then incubated at room temperature for 25 minutes. Following incubation, the Solution 1 and 2 mixture was added dropwise to each well of a 96-well plate containing the cells. Approximately 72 hours post transfection, cells were trypsinized by adding TrypLE™ (Thermo Fisher Scientific) to the center of each well and incubating at 37° C. for approximately 5 minutes. D10 media was then added to each well and mixed to resuspend cells. The resuspended cells were centrifuged for 10 minutes to obtain a pellet, and the supernatant was discarded. The cell pellet was then resuspended in QuickExtract™ buffer (Lucigen©), and cells were incubated at 65° C. for 15 minutes, 68° C. for 15 minutes, and 98° C. for 10 minutes.

Next Generation Sequencing (NGS) samples were prepared by two rounds of PCR. Three technical replicates were analyzed per target for the reference and each variant. The first round (PCR1) was used to amplify specific genomic regions depending on the target. Round 2 PCR (PCR2) was performed to add Illumina adapters and indices. Reactions were then pooled and purified by column purification. Sequencing runs were performed using a kit such as a 150 Cycle NextSeq® 500/550 Mid or High Output® v2.5 Kit or a 200 Cycle NovaSeq® 6000 SP or S1 Reagent Kit v1.5.

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides are mismatches were filtered out as well. Up to 50,000 reads passing those filters were used for analysis, and reads were counted as an indel read if they contained an insertion or deletion. The QC standard for the minimum number of reads passing filters was 10,000.

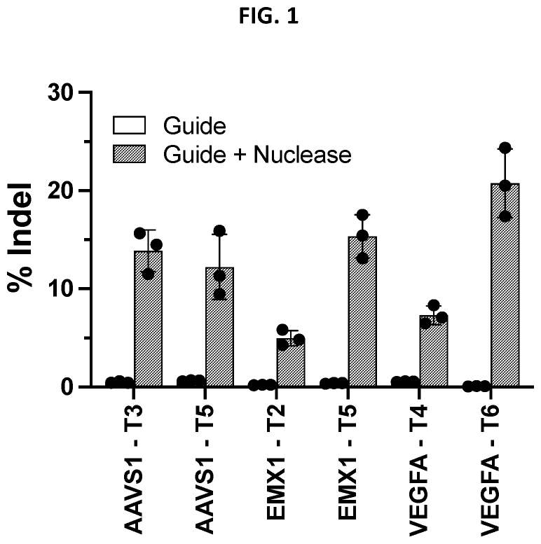

For each target, indel ratios, referring to the percentage of NGS reads comprising indels, were calculated for each sample and its cognate no protein control. Targets comprising a higher percentage of indels when Nuclease W was included in the transfection were indicative of DNA editing outcomes in the cell. As shown in FIG. 1, each of the six targets tested demonstrated a greater level of indels observed when the Nuclease W plasmid was present. This Example thus shows that Nuclease W (SEQ ID NO: 1) edited human genes.

### Example 2: Effectiveness of Variant CRISPR Nucleases for Targeting of Mammalian Genes

This Example describes indel assessment on mammalian targets using Nuclease W variants transfected into HEK293T cells following the methods disclosed in Example 1 above.

Arginine scanning mutagenesis was performed to individually substitute each non-arginine residue of Nuclease W (SEQ ID NO: 1) to arginine. SEQ ID NO: 1 is referred to herein as the reference sequence. This resulted in 858 single arginine substitution variants. Nucleic acids encoding the reference Nuclease W and each Nuclease W variant were then individually cloned into a pcDNA3.1 backbone (Invitrogen™), and the plasmids were maxi-prepped and diluted. The plasmids comprised a CMV promoter, a first NLS (KRTADGSEFESPKKKRKV; SEQ ID NO: 3) upstream of the coding sequence, and an XTEN linker (SGGSSGGSSGSETPGTSESATPESSGGSSGGSS; SEQ ID NO: 5) followed by a second NLS (KRPAATKKAGQAKKKK; SEQ ID NO: 4) downstream of the coding sequence. See also Example 1 above.

The RNA guide and target sequences are shown in Table 3. RNA guides were cloned into a pUC19 backbone (New England Biolabs©) following the U6 PolIII promoter and terminated with a 6×polyT sequence. The U6 PolIII uses a +1 G at the start of the transcript (i.e., the 5′ end of the RNA) for more efficient transcription that is excluded from the sequences described in Table 11. The plasmids were then maxi-prepped and diluted.

TABLE 3
Mammalian Targets and Corresponding crRNAs

| Target | PAM | | |
|---|---|---|---|
| Identifier | Sequence | Target Sequence | RNA Guide Sequence* |
| AAVS1 | CAG | GGTGCGTCCTAGGTGTTC | GGUGCGUCCUAGGUGUUCACguuu |
| | | AC (SEQ ID NO: 11) | cagcuacccugaguuucucaacuccguugagacuu |
| | | | ccucagguucaagcugaaaagagcaucuguccgga |
| | | | aggaccacuccggguuagggcagacccggcucuug |
| | | | ggcuuccuuggucuuugacucuggaggaaaccuu |
| | | | ccggcgugucgcaagacaagaugugagccug |
| | | | (SEQ ID NO: 12) |
| EMX1 | AAA | TAAACTACAGTGGTGCCT | UAAACUACAGUGGUGCCUGGguuu |
| | | GG (SEQ ID NO: 17) | cagcuacccugaguuucucaacuccguugagacuu |
| | | | ccucagguucaagcugaaaagagcaucuguccgga |
| | | | aggaccacuccggguuagggcagacccggcucuug |
| | | | ggcuuccuuggucuuugacucuggaggaaaccuu |
| | | | ccggcgugucgcaagacaagaugugagccug |
| | | | (SEQ ID NO: 18) |
| VEGFA | GAG | CCCAGTATGGGCAAAGG | CCCAGUAUGGGCAAAGGAAAguuu |
| | | AAA (SEQ ID NO: 21) | cagcuacccugaguuucucaacuccguugagacuu |
| | | | ccucagguucaagcugaaaagagcaucuguccgga |
| | | | aggaccacuccggguuagggcagacccggcucuug |
| | | | ggcuuccuuggucuuugacucuggaggaaaccuu |
| | | | ccggcgugucgcaagacaagaugugagccug |
| | | | (SEQ ID NO: 22) |

*spacer in upper case; scaffold in lower case

Cells were transfected, and samples were prepared for NGS as described in Example 1. Indel ratios, referring to the percentage of NGS reads comprising indels, were calculated for the reference and for each variant. The indel ratios used for fold change calculations were the average of two technical replicates. To then calculate fold change in indel ratios, the indel ratio for each variant was divided by the indel ratio for the reference. Table 4 shows fold change in indel ratios for the target. Numbering is relative to the reference nuclease of SEQ ID NO: 1 (i.e., without an NLS).

As shown in Table 4, 7 variants with single arginine substitutions (left column) were characterized as yielding at least a 2× increase in indel ratio relative to the reference indel ratio, when averaged across all targets (right column).

TABLE 4
Fold Change in Indel Ratios*

| | | | | Average Fold |
|---|---|---|---|---|
| Substitution | AAVS1 | EMX1 | VEGFA | Change |
| S925R | 2.69 | 3.44 | 3.15 | 3.09 |
| E715R | 2.35 | 3.94 | 2.93 | 3.07 |
| S695R | 2.06 | 3.34 | 2.06 | 2.48 |
| N691R | 1.94 | 2.70 | 2.28 | 2.30 |
| A657R | 2.25 | 2.10 | 2.40 | 2.25 |
| V117R | 1.78 | 2.29 | 2.47 | 2.18 |
| E848R | 1.71 | 2.86 | 1.90 | 2.16 |

*Variant indel ratio/Reference indel ratio

206 variants were analyzed as having indel ratios 1×-2× of the reference indel ratios, when averaged across all targets: A801R, E114R, A861R, E702R, E116R, D479R, A65R, L134R, V51R, A854R, E102R, F118R, G294R, V717R, E277R, S905R, T483R, E711R, A380R, N131R, A173R, N698R, D707R, I794R, V85R, F859R, E386R, A177R, N288R, Q292R, E271R, S44R, E270R, E113R, A115R, D382R, E112R, E124R, E48R, L37R, Q769R, A694R, E139R, D267R, A68R, A478R, D475R, A743R, A770R, P106R, S76R, S127R, S747R, S666R, Q826R, S326R, V523R, L268R, E739R, A788R, Q811R, N605R, I143R, A896R, E518R, A742R, V856R, G108R, K930R, I746R, G783R, M56R, T272R, L833R, S764R, Q266R, Q245R, I273R, A265R, G858R, P417R, E71R, H69R, E897R, Q206R, A785R, A690R, K701R, S620R, P269R, S612R, K60R, T280R, T599R, G529R, Q389R, T733R, E864R, L75R, L264R, E109R, T59R, Q627R, S895R, S863R, E419R, K617R, V577R, 1953R, H911R, N432R, D759R, A603R, F260R, L248R, V88R, D777R, A688R, P532R, V548R, A613R, G947R, K751R, V718R, I610R, S835R, H151R, S468R, A684R, Q390R, S238R, L142R, Y394R, S210R, E135R, H635R, D39R, A621R, G579R, T420R, K455R, E680R, A891R, K710R, A456R, G263R, E602R, D189R, K45R, L214R, K405R, N408R, P705R, Q814R, V84R, K64R, V235R, K703R, L679R, Q624R, A460R, T324R, E274R, G395R, P951R, P857R, W609R, S457R, H665R, 1950R, Q871R, F120R, K397R, Q543R, S740R, D780R, D211R, A305R, E291R, A632R, K416R, D447R, E784R, K843R, Q894R, K480R, L400R, S293R, V615R, A726R, A772R, Q564R, T581R, G834R, Q648R, K482R, G314R, D156R, K14R, S461R, Y309R, G521R, L27R, L499R, H890R, and S262R. The remaining variants (645 variants) resulted in decreased indel ratios relative to the reference indel ratios (fold change in indel ratios of less than 1.0).

The following substitutions, which resulted in at least a 2×-increase in indel ratios relative to the reference (Nuclease W of SEQ ID NO: 1), were selected to engineer combination variants: S925R, E715R, S695R, N691R, A657R, V117R, and A65R.

### Example 3: Effectiveness of Combination CRISPR Nuclease Variants for Targeting of Mammalian Genes

This Example describes indel assessment on mammalian targets using Nuclease W variants comprising two or more substitutions identified as increasing indel activity in Example 2. 56 combination Nuclease W variants were tested.

Each Nuclease W variant was cloned as described in Example 2. HEK293T cells were further transfected with the Nuclease W variant and an RNA guide of Table 3 above, followed by NGS analysis, as described in Example 2. For each target, indel ratios, referring to the percentage of NGS reads comprising indels, were calculated for the reference Nuclease W (SEQ ID NO: 1) and for each Nuclease W variant. The indel ratios shown in Table 5 were calculated as the average of two bioreplicates, each of which contained two technical replicates.

TABLE 5
Fold Change in Indel Ratios*

| | | | | Average |
|---|---|---|---|---|
| | | | | Fold |
| Substitutions | AAVS1 | EMX1 | VEGFA | Change |
| S925R, E715R, A65R | 3.03 | 4.84 | 3.78 | 3.88 |
| S925R, A657R, A65R | 3.20 | 4.67 | 3.71 | 3.86 |
| S925R, S695R, A657R | 3.14 | 4.67 | 3.63 | 3.82 |
| S925R, S695R, N691R | 2.97 | 4.73 | 3.70 | 3.80 |
| S925R, N691R, A65R | 3.00 | 4.59 | 3.58 | 3.72 |
| S925R, S695R, V117R | 2.96 | 4.44 | 3.73 | 3.71 |
| S925R, E715R, N691R | 2.97 | 4.60 | 3.52 | 3.70 |
| S925R, E715R, V117R | 2.92 | 4.47 | 3.65 | 3.68 |
| S925R, E715R, S695R | 2.90 | 4.65 | 3.45 | 3.67 |
| S925R, S695R, A65R | 2.91 | 4.62 | 3.47 | 3.67 |
| S925R, N691R, A657R | 3.03 | 4.45 | 3.39 | 3.62 |
| S695R, N691R, A657R | 2.99 | 4.33 | 3.46 | 3.59 |
| E715R, V117R, A65R | 2.73 | 4.49 | 3.54 | 3.59 |
| S925R, A657R, V117R | 3.03 | 4.13 | 3.56 | 3.57 |
| E715R, S695R, V117R | 2.81 | 4.38 | 3.49 | 3.56 |
| S925R, S695R | 2.81 | 4.28 | 3.53 | 3.54 |
| E715R, A657R, A65R | 2.96 | 4.30 | 3.29 | 3.52 |
| S925R, E715R | 2.77 | 4.25 | 3.50 | 3.51 |
| S925R, E715R, A657R | 3.01 | 4.14 | 3.31 | 3.49 |
| S925R, N691R | 2.72 | 4.13 | 3.50 | 3.45 |
| S925R, V117R, A65R | 2.69 | 4.19 | 3.41 | 3.43 |
| E715R, S695R, N691R | 2.89 | 4.17 | 3.23 | 3.43 |
| E715R, S695R, A65R | 2.68 | 4.46 | 3.12 | 3.42 |
| S925R, N691R, V117R | 2.81 | 3.95 | 3.42 | 3.39 |
| S925R, A657R | 2.83 | 3.79 | 3.47 | 3.36 |
| S925R, V117R | 2.70 | 3.66 | 3.68 | 3.35 |
| E715R, S695R, A657R | 2.88 | 4.18 | 2.96 | 3.34 |
| E715R, N691R, A657R | 2.86 | 4.13 | 3.03 | 3.34 |
| E715R, A657R, V117R | 2.84 | 3.88 | 3.28 | 3.33 |
| E715R, N691R | 2.60 | 4.06 | 3.18 | 3.28 |
| N691R, A657R | 2.82 | 3.68 | 3.30 | 3.27 |
| S695R, N691R, A65R | 2.66 | 4.09 | 3.06 | 3.27 |
| E715R, A657R | 2.82 | 3.72 | 3.25 | 3.26 |
| N691R, A657R, A65R | 2.75 | 3.95 | 3.06 | 3.25 |
| E715R, N691R, A65R | 2.54 | 4.13 | 3.07 | 3.25 |
| E715R, S695R | 2.53 | 4.14 | 3.04 | 3.24 |
| E715R, V117R | 2.47 | 3.79 | 3.45 | 3.24 |
| A657R, V117R, A65R | 2.74 | 3.74 | 3.23 | 3.24 |
| S695R, N691R, V117R | 2.61 | 3.67 | 3.40 | 3.23 |
| S695R, A657R, A65R | 2.80 | 3.88 | 2.94 | 3.21 |
| N691R, V117R, A65R | 2.50 | 3.94 | 3.15 | 3.20 |
| S695R, A657R, V117R | 2.76 | 3.45 | 3.28 | 3.16 |
| N691R, A657R, V117R | 2.77 | 3.49 | 3.20 | 3.15 |
| S925R, A65R | 2.55 | 3.60 | 3.06 | 3.07 |
| E715R, N691R, V117R | 2.46 | 3.63 | 2.98 | 3.02 |
| S695R, N691R | 2.44 | 3.61 | 2.93 | 2.99 |
| S695R, V117R, A65R | 2.30 | 3.70 | 2.95 | 2.98 |
| E715R, A65R | 2.44 | 3.68 | 2.77 | 2.97 |
| N691R, V117R | 2.32 | 3.27 | 3.22 | 2.94 |
| N691R, A65R | 2.36 | 3.63 | 2.73 | 2.91 |
| S695R, A657R | 2.64 | 3.11 | 2.91 | 2.89 |
| A657R, V117R | 2.46 | 2.63 | 3.16 | 2.75 |
| V117R, A65R | 2.02 | 3.23 | 2.84 | 2.70 |
| A657R, A65R | 2.57 | 2.93 | 2.56 | 2.69 |
| S695R, V117R | 2.02 | 3.01 | 3.00 | 2.68 |
| S695R, A65R | 2.04 | 3.00 | 1.92 | 2.32 |

*Variant indel ratio/Reference indel ratio

As shown in Table 5, all 56 Nuclease W variants with combinations of amino acid substitutions exhibited higher indel activity than Nuclease W (SEQ ID NO: 1). 18 Nuclease W variants were analyzed as yielding at least a 3.5× increase in indel ratio relative to the reference indel ratio, when averaged across all targets: a) S925R, E715R, and A65R, b) S925R, A657R, and A65R, c) S925R, S695R, and A657R, d) S925R, S695R, and N691R, e) S925R, N691R, and A65R, f) S925R, S695R, and V117R, g) S925R, E715R, and N691R, h) S925R, E715R, and V117R, I) S925R, E715R, and S695R, j) S925R, S695R, and A65R, k) S925R, N691R, and A657R, l) S695R, N691R, and A657R, m) E715R, V117R, and A65R, n) S925R, A657R, and V117R, o) E715R, S695R, and V117R, p) S925R and S695R, q) E715R, A657R, and A65R, and r) S925R and E715R. The remaining variants (38) were analyzed as having indel ratios 2×-3.5× of the reference indel ratios, when averaged across all targets.

The two Nuclease W variants that resulted in the highest increase in indel ratio relative to the reference indel ratio were selected for further experimentation. These Nuclease W variants comprised the following substitutions relative to SEQ ID NO: 1: a) S925R, E715R, and A65R and b) S925R, A657R, and A65R.

### Example 4: Engineering and Effectiveness of CRISPR Nickase Variants for Targeting Mammalian Genes

This Example describes introducing mutations into the CRISPR nuclease of SEQ ID NO: 1 (Nuclease W) that disrupt either the HNH or RuvC domains to produce a functional nickase. E425, H426, and N449 were identified as putative catalytic residues of the HNH domain. D10, E362, and D487 positions were identified as putative catalytic residues of the RuvC domain. These positions were identified by analyzing models generated with AlphaFold2 (Jumper et al., Nature 596: 583-9 (2021)) for structural regions resembling known HNH and RuvC active sites and/or by performing sequence alignments to other nucleases for which candidate positions had been previously identified. Examples of reference structures used to identify the HNH and RuvC active sites are represented with the following PDB IDs: ShOm, 7eu9, 61tu, 7odf, 7lys, 8dc2, 4cmp, 4oo8, 7z4j, 5axw, 5b2o, 6kc8, 7utn, 8csz, 8ctl, 8dmb.

The coding sequence of Nuclease W was converted into an E. coli-codon optimized DNA sequence, synthesized, and cloned into a pET-28a(+) vector (Novagen) containing lac and T7 RNA polymerase promoters for gene expression. To test for nickase activity, individual alanine mutants were cloned for each of the positions identified as putative active site residues of the HNH and RuvC domains. A leucine mutant was also cloned for position H426. Research grade plasmids were received from GenScript. The engineered nickase sequences are shown in Table 6. The codon encoding the substituted residue is capitalized, bold, and underlined in the nucleotide sequence, and the substituted residue is shown in bold and underlined in the amino acid sequence. The putative HNH-knockout nickases were anticipated to cleave the non-target strand but not the target strand. The putative RuvC-knockout nickases were anticipated to cleave the target strand but not the non-target strand.

TABLE 6
CRISPR Nuclease and Nickase Sequences

| CRISPR | | |
|---|---|---|
| Nuclease- | Nucleotide sequence | |
| Nickase | (codon optimized for E. coli cells) | Amino acid sequence |
| Reference | atgagttcaacacacactctagctatagatttcggtagcaaaaacattgg | SEQ ID NO: 1 |
| Nuclease W | catcgctctggtgcgcaaccaggacagccttaacaccccgctgttcgcg | |
| | ggtaccgcactgtacgaccggtttaccctgtcgaaaaaagtggagcca | |
| | cgtgttcagatgcgacgcatgcgtcggacgaaaaagacccgtaaggct | |
| | agattggcacatctggaagcgggattgctgagccacggtctggagccg | |
| | gacgtcgtcgtttggctggttaatttctgcaggaggagaggcttcaagag | |
| | cctgttcgatgaaggtcgtgaaccacgtggtgaaaaaaaagaggagga | |
| | ggcggaagtgttccgttttagccgtgaggaattcttcagcgcgttagaga | |
| | acgaactgctggagcgcctgccggaagagaagcttatcccggttctag | |
| | acctctgcgaacacgggttgaaccgtgatggcgatcgctttaaggaggt | |
| | tagattgatccgtattgataaccgtggtgcttcccgttgtgcatgggatgg | |
| | ctgcaatagcgtcaccccgcgtagagacaatgcactacgtgatgcactg | |
| | gcgcagttcgtttttacggtgtatgctcaaaaggtgcgctccgatccgga | |
| | tctgtacgaaaaaattcagcgcatgttagatcaactggcccagctaggta | |
| | agcgtctgcgccacgttggtggttccgacccgaaaaaagagcgtcaag | |
| | ttctgctgaagcgcatccgtgaagagctgaagttgctcaaattcctgtctg | |
| | gcttagcccaggacctgccggaagaaacgatcgaaccgaaagaggc | |
| | ctggacccatattcgtcgtaacctgatgaatctgattgagcagagcggg | |
| | ggccgtaaccgtttttgccgtcatcatagcgcgcaatatgtttaccatctg | |
| | atcgagggcaaaccgatcccgtttaagagatctcttaccgaaagcgaca | |
| | tgacgagccgtcgtgaagagattctgtttcaaaaactgtggcgttatatcg | |
| | aagcccgtgtgctcccgctggctccagcgggcatcgatagagtcgtgg | |
| | tggaacgaaccgcgtttgacatgctggcgggtagccgtaaacagcgtc | |
| | aaaaagttgcggacgactatttggaggaaatgtatcagcagggtccgc | |
| | gttacggctttaaggacgatctggaaatgctgcgcaaggaattcaacgg | |
| | tttgtgcgcatactgcggtaagccgagcgagaccattgttgaacgcgaa | |
| | cacattctgccgaaagcgaacttcttcttcgacagctatctgaacalggtt | |
| | ccggcgtgcccggactgtaaccgtatgtggaaagccaaggcgtcccc | |
| | gggtgcaagcggtttacgtatcaacgagagtgcataccaagcttacagc | |
| | gattacctggctgacaagtttaagacccgtccaccgcatttgttccacac | |
| | gattaaaaaaggaattctgaagctgatgacccagaaagatcgcgtgttg | |
| | gaagcggagaggttgctgggcatgatcgctgaaaacttgggccgtgtt | |
| | gtggagagccagcgtggcccgcgtccgctggctcgctacctgtgtgaa | |
| | aagctccgtcaacgttacgatcgtgtgacccagatcgcatttcgttctggt | |
| | agacacacggcgatctggcgtcaagcggcttacccggactttgataaa | |
| | gcgaaagagaaagtgtcgggtggaaccatcaatcatgcattggacgcc | |
| | atgattctggcctgcgatttgccagacgttaccgcccttgaggcgcgca | |
| | acctgccaccgtggatcatttctgcgtgggttgataaagttcgttccgcg | |
| | gcaccgcaggctggccaagagggcattccggccatcccgcatccgga | |
| | ctttgcggtaccggaatttgaaaaagttttacaaggtaacttcatcgaaac | |
| | cgacttggctaagctgaactggaaccgcaagcacagccgtgtgcagc | |
| | gccaagatgcatacggctggtgtggtctggaggacgtgccggctaagc | |
| | gcatcgccgcggcgaatctggcggcgtccctccgcaacgcagataag | |
| | gagaaaactccggaggaccgccaaaaggaggtgaaaaaagaagtgg | |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc (SEQ ID NO: 23) | |
| E425A | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| HNH- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVVE |
| | gcttagcccaggacctgccggaagaaacgatcgaaccgaaagaggc | RTAFDMLAGSRKQRQKVADDY |
| | ctggacccatattcgtcgtaacctgatgaatctgattgagcagagcggg | LEEMYQQGPRYGFKDDLEMLR |
| | ggccgtaaccgtttttgccgtcatcatagcgcgcaatatgtttaccatctg | KEFNGLCAYCGKPSETIVERAHI |
| | atcgagggcaaaccgatcccgtttaagagatctcttaccgaaagcgaca | LPKANFFFDSYLNMVPACPDCN |
| | tgacgagccgtcgtgaagagattctgtttcaaaaactgtggcgttatatcg | RMWKAKASPGASGLRINESAYQ |
| | aagcccgtgtgctcccgctggctccagcgggcatcgatagagtcgtgg | AYSDYLADKFKTRPPHLFHTIKK |
| | tggaacgaaccgcgtttgacatgctggcgggtagccgtaaacagcgtc | GILKLMTQKDRVLEAERLLGMI |
| | aaaaagttgcggacgactatttggaggaaatgtatcagcagggtccgc | AENLGRVVESQRGPRPLARYLC |
| | gttacggctttaaggacgatctggaaatgctgcgcaaggaattcaacgg | EKLRQRYDRVTQIAFRSGRHTAI |
| | tttgtgcgcatactgcggtaagccgagcgagaccattgttgaacgcGC | WRQAAYPDFDKAKEKVSGGTIN |
| | Gcacattctgccgaaagcgaacttcttcttcgacagctatctgaacatgg | HALDAMILACDLPDVTALEARN |
| | ttccggcgtgcccggactgtaaccgtatgtggaaagccaaggcgtccc | LPPWIISAWVDKVRSAAPQAGQ |
| | cgggtgcaagcggtttacgtatcaacgagagtgcataccaagcttacag | EGIPAIPHPDFAVPEFEKVLQGNF |
| | cgattacctggctgacaagtttaagacccgtccaccgcatttgttccaca | IETDLAKLNWNRKHSRVQRQDA |
| | cgattaaaaaaggaattctgaagctgatgacccagaaagatcgcgtgtt | YGWCGLEDVPAKRIAAANLAAS |
| | ggaagcggagaggttgctgggcatgatcgctgaaaacttgggccgtgt | LRNADKEKTPEDRQKEVKKEVV |
| | tgtggagagccagcgtggcccgcgtccgctggctcgctacctgtgtga | VVVHPRLRAVLEAANTGERPGE |
| | aaagctccgtcaacgttacgatcgtgtgacccagatcgcatttcgttctg | SSAAALISWLRKAVKGSLKDAR |
| | gtagacacacggcgatctggcgtcaagcggcttacccggactttgataa | LSSHPADQARAKALRDFADGTG |
| | agcgaaagagaaagtgtcgggtggaaccatcaatcatgcattggacgc | EAIPAVIGVRIRYPWLKANMDLH |
| | catgattctggcctgcgatttgccagacgttaccgcccttgaggcgcgc | RVERQTGQIVHRYVADPANQAL |
| | aacctgccaccgtggatcatttctgcgtgggttgataaagttcgttccgcg | IVAYLGSNGKVRRDKPMTLELR |
| | gcaccgcaggctggccaagagggcattccggccatcccgcatccgga | QSGAVVPGFKALSEVPAGPLQG |
| | ctttgcggtaccggaatttgaaaaagttttacaaggtaacttcatcgaaac | RALGQPRPDPGEWQAALHAYLQ |
| | cgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 25) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 24) | |
| H426A | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| HNH- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVVE |
| | gcttagcccaggacctgccggaagaaacgatcgaaccgaaagaggc | RTAFDMLAGSRKQRQKVADDY |
| | ctggacccatattcgtcgtaacctgatgaatctgattgagcagagcggg | LEEMYQQGPRYGFKDDLEMLR |
| | ggccgtaaccgtttttgccgtcatcatagcgcgcaatatgtttaccatctg | KEFNGLCAYCGKPSETIVEREAI |
| | atcgagggcaaaccgatcccgtttaagagatctcttaccgaaagcgaca | LPKANFFFDSYLNMVPACPDCN |
| | tgacgagccgtcgtgaagagattctgtttcaaaaactgtggcgttatatcg | RMWKAKASPGASGLRINESAYQ |
| | aagcccgtgtgctcccgctggctccagcgggcatcgatagagtcgtgg | AYSDYLADKFKTRPPHLFHTIKK |
| | tggaacgaaccgcgtttgacatgctggcgggtagccgtaaacagcgtc | GILKLMTQKDRVLEAERLLGMI |
| | aaaaagttgcggacgactatttggaggaaatgtatcagcagggtccgc | AENLGRVVESQRGPRPLARYLC |
| | gttacggctttaaggacgatctggaaatgctgcgcaaggaattcaacgg | EKLRQRYDRVTQIAFRSGRHTAI |
| | tttgtgcgcatactgcggtaagccgagcgagaccattgttgaacgcgaa | WRQAAYPDFDKAKEKVSGGTIN |
| | GCGattctgccgaaagcgaacttcttcttcgacagctatctgaacatg | HALDAMILACDLPDVTALEARN |
| | gttccggcgtgcccggactgtaaccgtatgtggaaagccaaggcgtcc | LPPWIISAWVDKVRSAAPQAGQ |
| | ccgggtgcaagcggtttacgtatcaacgagagtgcataccaagcttaca | EGIPAIPHPDFAVPEFEKVLQGNF |
| | gcgattacctggctgacaagtttaagacccgtccaccgcatttgttccac | IETDLAKLNWNRKHSRVQRQDA |
| | acgattaaaaaaggaattctgaagctgatgacccagaaagatcgcgtgt | YGWCGLEDVPAKRIAAANLAAS |
| | tggaagcggagaggttgctgggcatgatcgctgaaaacttgggccgtg | LRNADKEKTPEDRQKEVKKEVV |
| | ttgtggagagccagcgtggcccgcgtccgctggctcgctacctgtgtga | VVVHPRLRAVLEAANTGERPGE |
| | aaagctccgtcaacgttacgatcgtgtgacccagatcgcatttcgttctg | SSAAALISWLRKAVKGSLKDAR |
| | gtagacacacggcgatctggcgtcaagcggcttacccggactttgataa | LSSHPADQARAKALRDFADGTG |
| | agcgaaagagaaagtgtcgggtggaaccatcaatcatgcattggacgc | EAIPAVIGVRIRYPWLKANMDLH |
| | catgattctggcctgcgatttgccagacgttaccgcccttgaggcgcgc | RVERQTGQIVHRYVADPANQAL |
| | aacctgccaccgtggatcatttctgcgtgggttgataaagttcgttccgcg | IVAYLGSNGKVRRDKPMTLELR |
| | gcaccgcaggctggccaagagggcattccggccatcccgcatccgga | QSGAVVPGFKALSEVPAGPLQG |
| | ctttgcggtaccggaatttgaaaaagttttacaaggtaacttcatcgaaac | RALGQPRPDPGEWQAALHAYLQ |
| | cgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 27) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 26) | |
| H426L | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| HNH- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVVE |
| | gcttagcccaggacctgccggaagaaacgatcgaaccgaaagaggc | RTAFDMLAGSRKQRQKVADDY |
| | ctggacccatattcgtcgtaacctgatgaatctgattgagcagagcggg | LEEMYQQGPRYGFKDDLEMLR |
| | ggccgtaaccgtttttgccgtcatcatagcgcgcaatatgtttaccatctg | KEFNGLCAYCGKPSETIVERELIL |
| | atcgagggcaaaccgatcccgtttaagagatctcttaccgaaagcgaca | PKANFFFDSYLNMVPACPDCNR |
| | tgacgagccgtcgtgaagagattctgtttcaaaaactgtggcgttatatcg | MWKAKASPGASGLRINESAYQA |
| | aagcccgtgtgctcccgctggctccagcgggcatcgatagagtcgtgg | YSDYLADKFKTRPPHLFHTIKKG |
| | tggaacgaaccgcgtttgacatgctggcgggtagccgtaaacagcgtc | ILKLMTQKDRVLEAERLLGMIAE |
| | aaaaagttgcggacgactatttggaggaaatgtatcagcagggtccgc | NLGRVVESQRGPRPLARYLCEK |
| | gttacggctttaaggacgatctggaaatgctgcgcaaggaattcaacgg | LRQRYDRVTQIAFRSGRHTAIWR |
| | tttgtgcgcatactgcggtaagccgagcgagaccattgttgaacgcgaa | QAAYPDFDKAKEKVSGGTINHA |
| | CTGattctgccgaaagcgaacttcttcttcgacagctatctgaacatgg | LDAMILACDLPDVTALEARNLPP |
| | ttccggcgtgcccggactgtaaccgtatgtggaaagccaaggcgtccc | WIISAWVDKVRSAAPQAGQEGIP |
| | cgggtgcaagcggtttacgtatcaacgagagtgcataccaagcttacag | AIPHPDFAVPEFEKVLQGNFIETD |
| | cgattacctggctgacaagtttaagacccgtccaccgcatttgttccaca | LAKLNWNRKHSRVQRQDAYGW |
| | cgattaaaaaaggaattctgaagctgatgacccagaaagatcgcgtgtt | CGLEDVPAKRIAAANLAASLRN |
| | ggaagcggagaggttgctgggcatgatcgctgaaaacttgggccgtgt | ADKEKTPEDRQKEVKKEVVVV |
| | tgtggagagccagcgtggcccgcgtccgctggctcgctacctgtgtga | VHPRLRAVLEAANTGERPGESS |
| | aaagctccgtcaacgttacgatcgtgtgacccagatcgcatttcgttctg | AAALISWLRKAVKGSLKDARLS |
| | gtagacacacggcgatctggcgtcaagcggcttacccggactttgataa | SHPADQARAKALRDFADGTGEA |
| | agcgaaagagaaagtgtcgggtggaaccatcaatcatgcattggacgc | IPAVIGVRIRYPWLKANMDLHR |
| | catgattctggcctgcgatttgccagacgttaccgcccttgaggcgcgc | VERQTGQIVHRYVADPANQALI |
| | aacctgccaccgtggatcatttctgcgtgggttgataaagttcgttccgcg | VAYLGSNGKVRRDKPMTLELRQ |
| | gcaccgcaggctggccaagagggcattccggccatcccgcatccgga | SGAVVPGFKALSEVPAGPLQGR |
| | ctttgcggtaccggaatttgaaaaagttttacaaggtaacttcatcgaaac | ALGQPRPDPGEWQAALHAYLQS |
| | cgacttggctaagctgaactggaaccgcaagcacagccgtgtgcagc | AEVAEYAMVSQGCVVHYEDGS |
| | gccaagatgcatacggctggtgtggtctggaggacgtgccggctaagc | ERYIRNFSSSYGFKKSLFKGMVG |
| | gcatcgccgcggcgaatctggcggcgtccctccgcaacgcagataag | VRRSPLSGRVIPNIRI (SEQ ID |
| | gagaaaactccggaggaccgccaaaaggaggtgaaaaaagaagtgg | NO: 29) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 28) | |
| N449A | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| HNH- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVVE |
| | gcttagcccaggacctgccggaagaaacgatcgaaccgaaagaggc | RTAFDMLAGSRKQRQKVADDY |
| | ctggacccatattcgtcgtaacctgatgaatctgattgagcagagcggg | LEEMYQQGPRYGFKDDLEMLR |
| | ggccgtaaccgtttttgccgtcatcatagcgcgcaatatgtttaccatctg | KEFNGLCAYCGKPSETIVEREHI |
| | atcgagggcaaaccgatcccgtttaagagatctcttaccgaaagcgaca | LPKANFFFDSYLNMVPACPDCA |
| | tgacgagccgtcgtgaagagattctgtttcaaaaactgtggcgttatatcg | RMWKAKASPGASGLRINESAYQ |
| | aagcccgtgtgctcccgctggctccagcgggcatcgatagagtcgtgg | AYSDYLADKFKTRPPHLFHTIKK |
| | tggaacgaaccgcgtttgacatgctggcgggtagccgtaaacagcgtc | GILKLMTQKDRVLEAERLLGMI |
| | aaaaagttgcggacgactatttggaggaaatgtatcagcagggtccgc | AENLGRVVESQRGPRPLARYLC |
| | gttacggctttaaggacgatctggaaatgctgcgcaaggaattcaacgg | EKLRQRYDRVTQIAFRSGRHTAI |
| | tttgtgcgcatactgcggtaagccgagcgagaccattgttgaacgcgaa | WRQAAYPDFDKAKEKVSGGTIN |
| | cacattctgccgaaagcgaacttcttcttcgacagctatctgaacatggtt | HALDAMILACDLPDVTALEARN |
| | ccggcgtgcccggactgtGCGcgtatgtggaaagccaaggcgtcc | LPPWIISAWVDKVRSAAPQAGQ |
| | ccgggtgcaagcggtttacgtatcaacgagagtgcataccaagcttaca | EGIPAIPHPDFAVPEFEKVLQGNF |
| | gcgattacctggctgacaagtttaagacccgtccaccgcatttgttccac | IETDLAKLNWNRKHSRVQRQDA |
| | acgattaaaaaaggaattctgaagctgatgacccagaaagatcgcgtgt | YGWCGLEDVPAKRIAAANLAAS |
| | tggaagcggagaggttgctgggcatgatcgctgaaaacttgggccgtg | LRNADKEKTPEDRQKEVKKEVV |
| | ttgtggagagccagcgtggcccgcgtccgctggctcgctacctgtgtga | VVVHPRLRAVLEAANTGERPGE |
| | aaagctccgtcaacgttacgatcgtgtgacccagatcgcatttcgttctg | SSAAALISWLRKAVKGSLKDAR |
| | gtagacacacggcgatctggcgtcaagcggcttacccggactttgataa | LSSHPADQARAKALRDFADGTG |
| | agcgaaagagaaagtgtcgggtggaaccatcaatcatgcattggacgc | EAIPAVIGVRIRYPWLKANMDLH |
| | catgattctggcctgcgatttgccagacgttaccgcccttgaggcgcgc | RVERQTGQIVHRYVADPANQAL |
| | aacctgccaccgtggatcatttctgcgtgggttgataaagttcgttccgcg | IVAYLGSNGKVRRDKPMTLELR |
| | gcaccgcaggctggccaagagggcattccggccatcccgcatccgga | QSGAVVPGFKALSEVPAGPLQG |
| | ctttgcggtaccggaatttgaaaaagttttacaaggtaacttcatcgaaac | RALGQPRPDPGEWQAALHAYLQ |
| | cgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 31) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 30) | |
| D10A | atgagttcaacacacactctagctataGCGttcggtagcaaaaacatt | MSSTHTLAIAFGSKNIGIALVRN |
| (putative | ggcatcgctctggtgcgcaaccaggacagccttaacaccccgctgttc | QDSLNTPLFAGTALYDRFTLSKK |
| RuvC- | gcgggtaccgcactgtacgaccggtttaccctgtcgaaaaaagtggag | VEPRVQMRRMRRTKKTRKARL |
| knockout) | ccacgtgttcagatgcgacgcatgcgtcggacgaaaaagacccgtaag | AHLEAGLLSHGLEPDVVVWLVN |
| | gctagattggcacatctggaagcgggattgctgagccacggtctggag | FCRRRGFKSLFDEGREPRGEKKE |
| | ccggacgtcgtcgtttggctggttaatttctgcaggaggagaggcttcaa | EEAEVFRFSREEFFSALENELLER |
| | gagcctgttcgatgaaggtcgtgaaccacgtggtgaaaaaaaagagga | LPEEKLIPVLDLCEHGLNRDGDR |
| | ggaggcggaagtgttccgttttagccgtgaggaattcttcagcgcgttag | FKEVRLIRIDNRGASRCAWDGC |
| | agaacgaactgctggagcgcctgccggaagagaagcttatcccggttc | NSVTPRRDNALRDALAQFVFTV |
| | tagacctctgcgaacacgggttgaaccgtgatggcgatcgctttaagga | YAQKVRSDPDLYEKIQRMLDQL |
| | ggttagattgatccgtattgataaccgtggtgcttcccgttgtgcatggga | AQLGKRLRHVGGSDPKKERQVL |
| | tggctgcaatagcgtcaccccgcgtagagacaatgcactacgtgatgc | LKRIREELKLLKFLSGLAQDLPE |
| | actggcgcagttcgtttttacggtgtatgctcaaaaggtgcgctccgatcc | ETIEPKEAWTHIRRNLMNLIEQS |
| | ggatctgtacgaaaaaattcagcgcatgttagatcaactggcccagcta | GGRNRFCRHHSAQYVYHLIEGK |
| | ggtaagcgtctgcgccacgttggtggttccgacccgaaaaaagagcgt | PIPFKRSLTESDMTSRREEILFQK |
| | caagttctgctgaagcgcatccgtgaagagctgaagttgctcaaattcct | LWRYIEARVLPLAPAGIDRVVVE |
| | gtctggcttagcccaggacctgccggaagaaacgatcgaaccgaaag | RTAFDMLAGSRKQRQKVADDY |
| | aggcctggacccatattcgtcgtaacctgatgaatctgattgagcagagc | LEEMYQQGPRYGFKDDLEMLR |
| | gggggccgtaaccgtttttgccgtcatcatagcgcgcaatatgtttaccat | KEFNGLCAYCGKPSETIVEREHI |
| | ctgatcgagggcaaaccgatcccgtttaagagatctcttaccgaaagcg | LPKANFFFDSYLNMVPACPDCN |
| | acatgacgagccgtcgtgaagagattctgtttcaaaaactgtggcgttat | RMWKAKASPGASGLRINESAYQ |
| | atcgaagcccgtgtgctcccgctggctccaggggcatcgatagagtc | AYSDYLADKFKTRPPHLFHTIKK |
| | gtggtggaacgaaccgcgtttgacatgctggcgggtagccgtaaacag | GILKLMTQKDRVLEAERLLGMI |
| | cgtcaaaaagttgcggacgactatttggaggaaatgtatcagcagggtc | AENLGRVVESQRGPRPLARYLC |
| | cgcgttacggctttaaggacgatctggaaatgctgcgcaaggaattcaa | EKLRQRYDRVTQIAFRSGRHTAI |
| | cggtttgtgcgcatactgcggtaagccgagcgagaccattgttgaacgc | WRQAAYPDFDKAKEKVSGGTIN |
| | gaacacattctgccgaaagcgaacttcttcttcgacagctatctgaacat | HALDAMILACDLPDVTALEARN |
| | ggttccggcgtgcccggactgtaaccgtatgtggaaagccaaggcgtc | LPPWIISAWVDKVRSAAPQAGQ |
| | cccgggtgcaagcggtttacgtatcaacgagagtgcataccaagcttac | EGIPAIPHPDFAVPEFEKVLQGNF |
| | agcgattacctggctgacaagtttaagacccgtccaccgcatttgttcca | IETDLAKLNWNRKHSRVQRQDA |
| | cacgattaaaaaaggaattctgaagctgatgacccagaaagatcgcgtg | YGWCGLEDVPAKRIAAANLAAS |
| | ttggaagcggagaggttgctgggcatgatcgctgaaaacttgggccgt | LRNADKEKTPEDRQKEVKKEVV |
| | gttgtggagagccagcgtggcccgcgtccgctggctcgctacctgtgt | VVVHPRLRAVLEAANTGERPGE |
| | gaaaagctccgtcaacgttacgatcgtgtgacccagatcgcatttcgttct | SSAAALISWLRKAVKGSLKDAR |
| | ggtagacacacggcgatctggcgtcaagcggcttacccggactttgata | LSSHPADQARAKALRDFADGTG |
| | aagcgaaagagaaagtgtcgggtggaaccatcaatcatgcattggacg | EAIPAVIGVRIRYPWLKANMDLH |
| | ccatgattctggcctgcgatttgccagacgttaccgcccttgaggcgcg | RVERQTGQIVHRYVADPANQAL |
| | caacctgccaccgtggatcatttctgcgtgggttgataaagttcgttccgc | IVAYLGSNGKVRRDKPMTLELR |
| | ggcaccgcaggctggccaagagggcattccggccatcccgcatccgg | QSGAVVPGFKALSEVPAGPLQG |
| | actttgcggtaccggaatttgaaaaagttttacaaggtaacttcatcgaaa | RALGQPRPDPGEWQAALHAYLQ |
| | ccgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 33) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 32) | |
| E362A | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| RuvC- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVV |
| | gcttagcccaggacctgccggaagaaacgatcgaaccgaaagaggc | ARTAFDMLAGSRKQRQKVADD |
| | ctggacccatattcgtcgtaacctgatgaatctgattgagcagagcggg | YLEEMYQQGPRYGFKDDLEML |
| | ggccgtaaccgtttttgccgtcatcatagcgcgcaatatgtttaccatctg | RKEFNGLCAYCGKPSETIVEREH |
| | atcgagggcaaaccgatcccgtttaagagatctcttaccgaaagcgaca | ILPKANFFFDSYLNMVPACPDCN |
| | tgacgagccgtcgtgaagagattctgtttcaaaaactgtggcgttatatcg | RMWKAKASPGASGLRINESAYQ |
| | aagcccgtgtgctcccgctggctccagcgggcatcgatagagtcgtgg | AYSDYLADKFKTRPPHLFHTIKK |
| | tgGCGcgaaccgcgtttgacatgctggcgggtagccgtaaacagcg | GILKLMTQKDRVLEAERLLGMI |
| | tcaaaaagttgcggacgactatttggaggaaatgtatcagcagggtccg | AENLGRVVESQRGPRPLARYLC |
| | cgttacggctttaaggacgatctggaaatgctgcgcaaggaattcaacg | EKLRQRYDRVTQIAFRSGRHTAI |
| | gtttgtgcgcatactgcggtaagccgagcgagaccattgttgaacgcga | WRQAAYPDFDKAKEKVSGGTIN |
| | acacattctgccgaaagcgaacttcttcttcgacagctatctgaacatggt | HALDAMILACDLPDVTALEARN |
| | tccggcgtgcccggactgtaaccgtatgtggaaagccaaggcgtcccc | LPPWIISAWVDKVRSAAPQAGQ |
| | gggtgcaagcggtttacgtatcaacgagagtgcataccaagcttacagc | EGIPAIPHPDFAVPEFEKVLQGNF |
| | gattacctggctgacaagtttaagacccgtccaccgcatttgttccacac | IETDLAKLNWNRKHSRVQRQDA |
| | gattaaaaaaggaattctgaagctgatgacccagaaagatcgcgtgttg | YGWCGLEDVPAKRIAAANLAAS |
| | gaagcggagaggttgctgggcatgatcgctgaaaacttgggccgtgtt | LRNADKEKTPEDRQKEVKKEVV |
| | gtggagagccagcgtggcccgcgtccgctggctcgctacctgtgtgaa | VVVHPRLRAVLEAANTGERPGE |
| | aagctccgtcaacgttacgatcgtgtgacccagatcgcatttcgttctggt | SSAAALISWLRKAVKGSLKDAR |
| | agacacacggcgatctggcgtcaagcggcttacccggactttgataaa | LSSHPADQARAKALRDFADGTG |
| | gcgaaagagaaagtgtcgggtggaaccatcaatcatgcattggacgcc | EAIPAVIGVRIRYPWLKANMDLH |
| | atgattctggcctgcgatttgccagacgttaccgcccttgaggcgcgca | RVERQTGQIVHRYVADPANQAL |
| | acctgccaccgtggatcatttctgcgtgggttgataaagttcgttccgcg | IVAYLGSNGKVRRDKPMTLELR |
| | gcaccgcaggctggccaagagggcattccggccatcccgcatccgga | QSGAVVPGFKALSEVPAGPLQG |
| | ctttgcggtaccggaatttgaaaaagttttacaaggtaacttcatcgaaac | RALGQPRPDPGEWQAALHAYLQ |
| | cgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 35) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 34) | |
| D487A | atgagttcaacacacactctagctatagatttcggtagcaaaaacattgg | MSSTHTLAIDFGSKNIGIALVRN |
| (putative | catcgctctggtgcgcaaccaggacagccttaacaccccgctgttcgcg | QDSLNTPLFAGTALYDRFTLSKK |
| RuvC- | ggtaccgcactgtacgaccggtttaccctgtcgaaaaaagtggagcca | VEPRVQMRRMRRTKKTRKARL |
| knockout) | cgtgttcagatgcgacgcatgcgtcggacgaaaaagacccgtaaggct | AHLEAGLLSHGLEPDVVVWLVN |
| | agattggcacatctggaagcgggattgctgagccacggtctggagccg | FCRRRGFKSLFDEGREPRGEKKE |
| | gacgtcgtcgtttggctggttaatttctgcaggaggagaggcttcaagag | EEAEVFRFSREEFFSALENELLER |
| | cctgttcgatgaaggtcgtgaaccacgtggtgaaaaaaaagaggagga | LPEEKLIPVLDLCEHGLNRDGDR |
| | ggcggaagtgttccgttttagccgtgaggaattcttcagcgcgttagaga | FKEVRLIRIDNRGASRCAWDGC |
| | acgaactgctggagcgcctgccggaagagaagcttatcccggttctag | NSVTPRRDNALRDALAQFVFTV |
| | acctctgcgaacacgggttgaaccgtgatggcgatcgctttaaggaggt | YAQKVRSDPDLYEKIQRMLDQL |
| | tagattgatccgtattgataaccgtggtgcttcccgttgtgcatgggatgg | AQLGKRLRHVGGSDPKKERQVL |
| | ctgcaatagcgtcaccccgcgtagagacaatgcactacgtgatgcactg | LKRIREELKLLKFLSGLAQDLPE |
| | gcgcagttcgtttttacggtgtatgctcaaaaggtgcgctccgatccgga | ETIEPKEAWTHIRRNLMNLIEQS |
| | tctgtacgaaaaaattcagcgcatgttagatcaactggcccagctaggta | GGRNRFCRHHSAQYVYHLIEGK |
| | agcgtctgcgccacgttggtggttccgacccgaaaaaagagcgtcaag | PIPFKRSLTESDMTSRREEILFQK |
| | ttctgctgaagcgcatccgtgaagagctgaagttgctcaaattcctgtctg | LWRYIEARVLPLAPAGIDRVVVE |
| | gcttagcccaggacctgccggaagaaacgatcgaaccgaaagaggc | RTAFDMLAGSRKQRQKVADDY |
| | ctggacccatattcgtcgtaacctgatgaatctgattgagcagagcggg | LEEMYQQGPRYGFKDDLEMLR |
| | ggccgtaaccgtttttgccgtcatcatagcgcgcaatatgtttaccatctg | KEFNGLCAYCGKPSETIVEREHI |
| | atcgagggcaaaccgatcccgtttaagagatctcttaccgaaagcgaca | LPKANFFFDSYLNMVPACPDCN |
| | tgacgagccgtcgtgaagagattctgtttcaaaaactgtggcgttatatcg | RMWKAKASPGASGLRINESAYQ |
| | aagcccgtgtgctcccgctggctccagcgggcatcgatagagtcgtgg | AYSDYLADKFKTRPPHLFHTIKK |
| | tggaacgaaccgcgtttgacatgctggcgggtagccgtaaacagcgtc | GILKLMTQKDRVLEAERLLGMI |
| | aaaaagttgcggacgactatttggaggaaatgtatcagcagggtccgc | AENLGRVVESQRGPRPLARYLC |
| | gttacggctttaaggacgatctggaaatgctgcgcaaggaattcaacgg | EKLRQRYDRVTQIAFRSGRHTAI |
| | tttgtgcgcatactgcggtaagccgagcgagaccattgttgaacgcgaa | WRQAAYPDFDKAKEKVSGGTIN |
| | cacattctgccgaaagcgaacttcttcttcgacagctatctgaacatggtt | HALAAMILACDLPDVTALEARN |
| | ccggcgtgcccggactgtaaccgtatgtggaaagccaaggcgtcccc | LPPWIISAWVDKVRSAAPQAGQ |
| | gggtgcaagcggtttacgtatcaacgagagtgcataccaagcttacagc | EGIPAIPHPDFAVPEFEKVLQGNF |
| | gattacctggctgacaagtttaagacccgtccaccgcatttgttccacac | IETDLAKLNWNRKHSRVQRQDA |
| | gattaaaaaaggaattctgaagctgatgacccagaaagatcgcgtgttg | YGWCGLEDVPAKRIAAANLAAS |
| | gaagcggagaggttgctgggcatgatcgctgaaaacttgggccgtgtt | LRNADKEKTPEDRQKEVKKEVV |
| | gtggagagccagcgtggcccgcgtccgctggctcgctacctgtgtgaa | VVVHPRLRAVLEAANTGERPGE |
| | aagctccgtcaacgttacgatcgtgtgacccagatcgcatttcgttctggt | SSAAALISWLRKAVKGSLKDAR |
| | agacacacggcgatctggcgtcaagcggcttacccggactttgataaa | LSSHPADQARAKALRDFADGTG |
| | gcgaaagagaaagtgtcgggtggaaccatcaatcatgcattgGCGg | EAIPAVIGVRIRYPWLKANMDLH |
| | ccatgattctggcctgcgatttgccagacgttaccgcccttgaggcgcg | RVERQTGQIVHRYVADPANQAL |
| | caacctgccaccgtggatcatttctgcgtgggttgataaagttcgttccgc | IVAYLGSNGKVRRDKPMTLELR |
| | ggcaccgcaggctggccaagagggcattccggccatcccgcatccgg | QSGAVVPGFKALSEVPAGPLQG |
| | actttgcggtaccggaatttgaaaaagttttacaaggtaacttcatcgaaa | RALGQPRPDPGEWQAALHAYLQ |
| | ccgacttggctaagctgaactggaaccgcaagcacagccgtgtgcagc | SAEVAEYAMVSQGCVVHYEDG |
| | gccaagatgcatacggctggtgtggtctggaggacgtgccggctaagc | SERYIRNFSSSYGFKKSLFKGMV |
| | gcatcgccgcggcgaatctggcggcgtccctccgcaacgcagataag | GVRRSPLSGRVIPNIRI (SEQ ID |
| | gagaaaactccggaggaccgccaaaaggaggtgaaaaaagaagtgg | NO: 37) |
| | ttgtcgttgtgcacccgcgtctacgtgctgttttggaggcggcgaatacc | |
| | ggtgagcgtccgggcgagtctagcgcggctgcgctcatcagctggctg | |
| | agaaaggccgtaaaaggttctctcaaggatgcgcgcctgtcatctcacc | |
| | cggcagaccaggcgcgtgcaaaggccctcagagattttgccgacggt | |
| | actggtgaggccataccggctgtgattggcgttcgtattcgctacccgtg | |
| | gctgaaagcgaacatggatttgcaccgcgtagagcgccaaaccggtca | |
| | aattgtgcaccgctacgtggctgatccggcgaaccaggccctcatcgtc | |
| | gcgtacctgggcagcaatggtaaggtgcgtcgagataaacctatgactt | |
| | tggaactgaggcagtcgggtgccgtggtgcccggcttcaaggcgctca | |
| | gcgaagttccggcgggccctctgcaaggtcgtgcattgggccagccg | |
| | cgtccggacccgggcgagtggcaggcggcgctgcacgcgtatctgca | |
| | atccgcggaggtggccgagtatgcaatggtttcccagggttgcgtcgtg | |
| | cactatgaggacggttcggaacgttatatccgtaatttctctagctcatatg | |
| | gttttaagaagagcctgttcaagggcatggtcggtgtgagacgctcccc | |
| | gctgtccggccgcgttattccgaatatccgcatc | |
| | (SEQ ID NO: 36) | |

A linear DNA template sequence encoding an RNA guide was designed and ordered (IDT) with a T7 promoter upstream and a T7Te terminator sequence downstream of the guide. The RNA guide was designed to be specific to a previously tested target sequence, described in Example 1, within the coding exon of VEGFA with a 5′-NRV-3′ PAM sequence (the PAM is 3′ of the target sequence). The sequence of the encoded RNA guide and its individual components are shown in Table 7. The T7 promoter uses a +1 G at the start of the transcript (i.e., the 5′ end of the RNA) for more efficient transcription. This +1 G is shown for SEQ ID NO: 40.

TABLE 7
RNA Sequences

| Components | RNA sequence | SEQ ID NO |
|---|---|---|
| Reference | guuucagcuacccugaguuucucaacuccguugagacuuccucagguucaagcugaa | 2 |
| sgRNA | aagagcaucuguccggaaggaccacuccggguuagggcagacccggcucuugggcu | |
| scaffold | uccuuggucuuugacucuggaggaaaccuuccggcgugucgcaagacaagauguga | |
| | gccug | |
| VEGFA-T6 | CCCAGUAUGGGCAAAGGAAA | 38 |
| spacer | | |
| T7Te | ggcucaccuucgggugggccuuucugcg | 39 |
| terminator | | |
| T7 transcribed | gCCCAGUAUGGGCAAAGGAAAguuucagcuacccugaguuucucaa | 40 |
| VEGFA-T6 | cuccguugagacuuccucagguucaagcugaaaagagcaucuguccggaaggaccac | |
| sgRNA | uccggguuagggcagacccggcucuugggcuuccuuggucuuugacucuggaggaa | |
| | accuuccggcgugucgcaagacaagaugugagccugggcucaccuucgggugggcc | |
| | uuucugcg | |

A DNA target was designed and ordered as a synthesized linear DNA fragment. The target sequence from VEGFA and 10 bases upstream and downstream within the exon was flanked by 100 bases of unrelated sequence upstream and 200 bases of unrelated sequence downstream. The extra sequence was added so that the cleaved and uncleaved products would separate well on a gel. The target and non-target strands were labelled with 5′ IR800 and 5′ IR700 labels, respectively, through PCR amplification using labelled primers. The sequences of the DNA target, the individual components of the DNA target, and the labelled PCR primers are in Table 8.

TABLE 8
Target gBlock and Primer Sequences

| Components | DNA sequence | SEQ ID NO |
|---|---|---|
| VEGFA-T6 target | CCCAGTATGGGCAAAGGAAA | 21 |
| VEGFA-T6 | TTCCTTGGTC | 41 |
| upstream 10 nt | | |
| VEGFA-T6 | GAGGTACAGA | 42 |
| downstream 10 nt | | |
| 100 nt upstream | gctcaaagctttagactggaggtaccgtagtactatgagcttaatcgcgctgagggtgat | 43 |
| sequence | ttgggtgttcgtctcatatcttcgattatcggacagtagt | |
| 200 nt | agtagtatcggaccggttggcgaaccacagcgtataacgagacatggactcgcattcc | 44 |
| downstream | ctaccttaagcgggcggttttggagctcggaattcatgtaaactcgcccgtttaacataat | |
| sequence | ctagatggatcttgcgcgcaccgcccgaacaccactcgccacaaaaaaccgccggaa | |
| | cgtccaaaagtacgggttttgctg | |
| Full target gblock | gctcaaagctttagactggaggtaccgtagtactatgagcttaatcgcgctgagggtgat | 45 |
| | ttgggtgttcgtctcatatcttcgattatcggacagtagtTTCCTTGGTCCCCA | |
| | GTATGGGCAAAGGAAAGAGGTACAGAagtagtatcggaccggt | |
| | tggcgaaccacagcgtataacgagacatggactcgcattccctaccttaagcgggcgg | |
| | ttttggagctcggaattcatgtaaactcgcccgtttaacataatctagatggatcttgcgcg | |
| | caccgcccgaacaccactcgccacaaaaaaccgccggaacgtccaaaagtacgggt | |
| | tttgctg | |
| Forward primer | 5IRD700/GCTCAAAGCTTTAGACTGGAGGTACCG | 46 |
| Reverse primer | /5IRD800/CAGCAAAACCCGTACTTTTGGACG | 47 |

Cleavage activity of the reference Nuclease W (SEQ ID NO: 1) and each of the putative nickases was assessed using in vitro cleavage assays. Each polypeptide was individually co-expressed with the RNA guide in vitro by incubating the plasmid encoding the protein of interest from Table 6 and linear DNA template for the T7 transcribed VEGFA-T6 sgRNA from Table 7 in a PURExpress® solution (NEB) containing SUPERase⋅In™ RNase Inhibitor (Invitrogen) for 2 hours at 37° C. The unpurified polypeptide/RNA solution was then diluted into a solution of 1×NEB Buffer 2 (NEB) containing approximately 1 ng/μl of the labelled DNA target amplicon. The solution was then incubated for 1 hour at 37° C. Reactions were stopped by incubating with RNase Cocktail™ (Invitrogen; approximately 1 U/μl final concentration) at 37° C. for 15 minutes, followed by incubating with Proteinase K (NEB; approximately 0.04 U/μl final concentration) at 55° C. for 30 minutes. The DNA was then purified using CleanNGS DNA & RNA Clean-Up Magnetic Beads (Bulldog Bio).

The cleaved and uncleaved products of the target and non-target strands were separated by running the samples on a 10% TBE-Urea PAGE gel. The gel was imaged using a LI-COR Odysssey M imaging system using the 800 nm and 700 nm channels to visualize the 5′ IR800 and 5′ IR700 labels on the target and non-target strands of the target DNA substrate. Band intensities were quantified using ImageJ software.

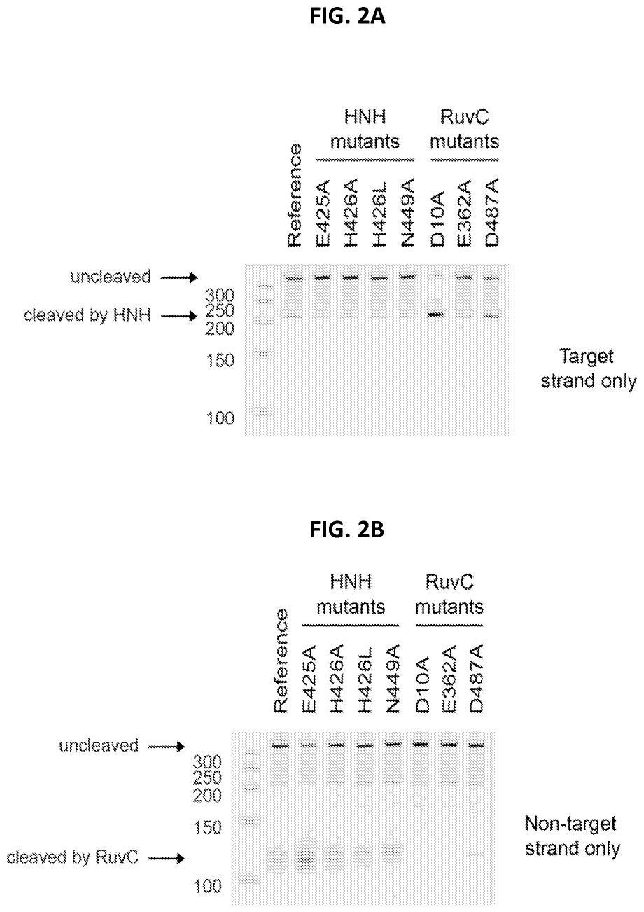

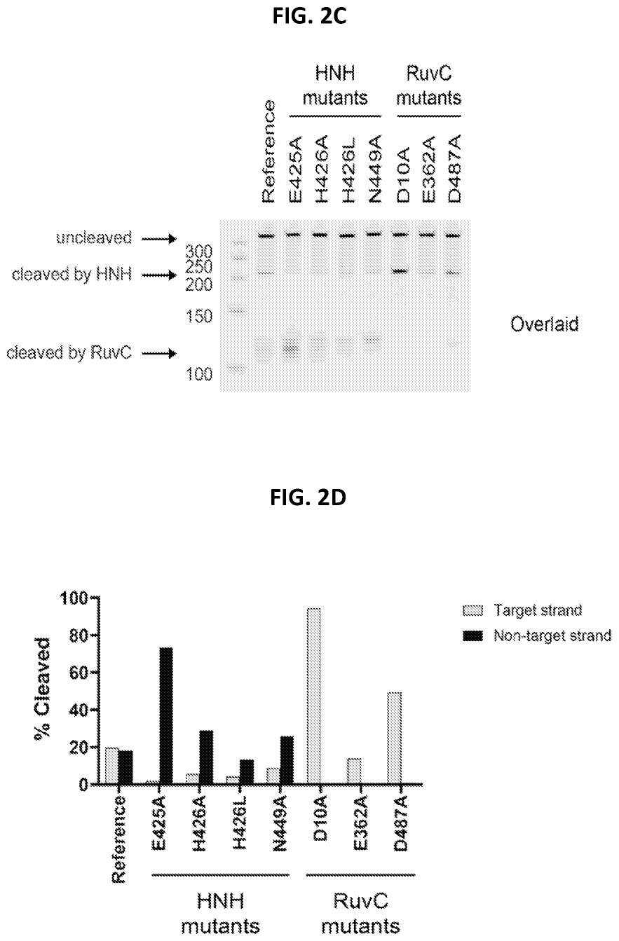

Gel images are shown in FIGS. 2A-2C, and quantification of the percent of cleaved target and non-target strands are shown in FIG. 2D. The uncleaved, HNH-cleaved, and RuvC-cleaved strands are indicated. FIG. 2A is a gel image captured using the 800 nm channel showing cleavage of the target strand. FIG. 2B is a gel image captured using the 700 nm channel showing cleavage of the non-target strand. FIG. 2C is an overlay of the gel images from FIG. 2A and FIG. 2B. As shown in FIG. 2A-D, the reference Nuclease W (SEQ ID NO: 1) cleaved both the target strand and the non-target strand, as expected. Each of the four HNH-knockout nickase constructs (E425A, H426A, H426L, and N449A) showed significantly decreased activity on the target strand while retaining activity that is similar to or greater than the reference protein on the non-target strand. Each of the three RuvC-knockout nickase constructs (D10A, E362A, and D487A) showed significantly decreased activity on the non-target strand while retaining activity on the target strand (FIG. 2A-2D).

This Example thus shows that HNH-knockout nickases and RuvC-knockout nickases were successfully engineered. The E425A variant of Nuclease W was chosen to install edits into human gene targets.

### Example 5: CRISPR Nuclease Mediated Editing of Human Target Genes in HEK293T Cells

This Example describes the genomic editing of the AAVS1, EMX1, and VEGFA genes by the CRISPR Nuclease S (SEQ ID NO: 49) introduced into cells by lipid-based transient transfection into the HEK293T cell line.

The CRISPR Nuclease S was tagged with N-terminal SV40 nuclear localization signal (NLS) and a C-terminal nucleoplasmin NLS, and its coding sequence was converted to a human codon-optimized DNA sequence, synthesized, and cloned into a pcDNA3.1 vector (Invitrogen), containing a CMV promoter for expression. The reference and NLS-tagged sequences used are in Table 9. Plasmids were purified using a midiprep kit.

RNA guides were designed and cloned and into a pUC19 plasmid following the U6 PolIII promoter and terminated with a 6×polyT sequence. RNA guides were designed to be specific to target sequences within the coding exons of AAVS1, EMX1, and VEGFA with 5′-ARCC-3′ PAM sequences (the PAM sequence is on the 3′ end of the target sequence). See all RNA guide sequences in Table 10. The U6 PolIII promoter uses a +1 G at the start of the transcript (i.e., the 5′ end of the RNA) for more efficient transcription that is excluded from the sequences described in Table 10. Plasmids were purified using a midiprep kit.

TABLE 9
Amino Acid Sequences

| Component | Amino acid sequence | SEQ ID NO |
|---|---|---|
| Nuclease S | MDREYLLEKIRDCNPDEIFTSFQIDTGAKYYGCTVLVKLPG | 49 |
| | VQKPIMVIADVLTAIPKIEDIVAARSLNRGTRRTNKSKKRR | |
| | NRKLRAAIGHIDGADKVLAYCKRRGVSYAFDQDEEETSAE | |
| | DKRTRKSREEVLDKLKVVVRNEIPDEQQDFVYKTCEKLLN | |
| | AEERKCRIKNRNPSKCDWEGCNQNVPASRNAIFETLEQTLY | |
| | PWLTQIFRSSKSYVRLENELNECVKYLNSLDYRNKYSIEQT | |
| | DDEEKQLDKQLNNELNKIRDIVKSHASKEHYREFSHYWTK | |
| | YYRKQLANILKSSGTGRTTFCRQHSSMYVAKLIAGEVIPVK | |
| | PEIYLKDIEGAAQKLTYAKLESYLLHSVLPTLKTIGITKIDSV | |
| | CVESASGSSPSKAKKQKRSDKAKKQKVSNKTKKQKCSDEE | |
| | FQQGPMFKFDSRKDMLLAEFDKLCAYCGEPLTGSYIENEHI | |
| | LPRSRFPYDSYFNILPACEKCNRRKGARTPYEAGMTISTKA | |
| | LDAFDRYIKKKKFVHPSIWVKRNLLSGMTHQTNGDAEREL | |
| | RFLCRKLFISGKHTQTPKRLARLIATSLSNATGIRPQIKSIPP | |
| | KFVYFYRNAVLPDFNKYEDKKNGGVVNHIIDSALLGCDFP | |
| | SPHYLLSGRYSGCEKYREFVDRLKDSFVAVDDSGMPICIPIR | |
| | SETQFFQEQIDKRLPGYSKIDMSKMNWNSKRKIVSHYQPQ | |
| | RKVGYKLVAARDSIESIKDRLLDLNKKQGTDTVIRDATIESI | |
| | RDEKLKSRMLNNPDRAAYYLIEYVQQSIKYGSNEEVYHPT | |
| | AIEMKQMRDEFVNDDPELYVTEDKELRKQVPNIFGMRIQT | |
| | GQSESQISFHRVCTQGDTVHHHHYMQDIGIRHIAIVVHIDK | |
| | KGKKQNQAITVTRQGKVYIGLGTARKSVDRSFGMTLQGR | |
| | AFGEKGSRKKWDRQWHREFRAFCKATWNVERIHILTAGC | |
| | VFIDTDGTTYFVRNMDSQDKGIRIATAKRKFVEIERVFKSP | |
| | HAYQKYLSKMNAITK | |
| SV40 NLS | KRTADGSEFESPKKKRKV | 3 |
| Nucleoplasmin | KRPAATKKAGQAKKKK | 4 |
| NLS | | |
| XTEN linker | SGGSSGGSSGSETPGTSESATPESSGGSSGGSS | 5 |
| NLS-tagged | MKRTADGSEFESPKKKRKVDREYLLEKIRDCNPDEIFTSFQID | 50 |
| Nuclease S | TGAKYYGCTVLVKLPGVQKPIMVIADVLTAIPKIEDIVAAR | |
| | SLNRGTRRTNKSKKRRNRKLRAAIGHIDGADKVLAYCKRR | |
| | GVSYAFDQDEEETSAEDKRTRKSREEVLDKLKVVVRNEIP | |
| | DEQQDFVYKTCEKLLNAEERKCRIKNRNPSKCDWEGCNQ | |
| | NVPASRNAIFETLEQTLYPWLTQIFRSSKSYVRLENELNECV | |
| | KYLNSLDYRNKYSIEQTDDEEKQLDKQLNNELNKIRDIVKS | |
| | HASKEHYREFSHYWTKYYRKQLANILKSSGTGRTTFCRQH | |
| | SSMYVAKLIAGEVIPVKPEIYLKDIEGAAQKLTYAKLESYL | |
| | LHSVLPTLKTIGITKIDSVCVESASGSSPSKAKKQKRSDKAK | |
| | KQKVSNKTKKQKCSDEEFQQGPMFKFDSRKDMLLAEFDK | |
| | LCAYCGEPLTGSYIENEHILPRSRFPYDSYFNILPACEKCNR | |
| | RKGARTPYEAGMTISTKALDAFDRYIKKKKFVHPSIWVKR | |
| | NLLSGMTHQTNGDAERELRFLCRKLFISGKHTQTPKRLARL | |
| | IATSLSNATGIRPQIKSIPPKFVYFYRNAVLPDFNKYEDKKN | |
| | GGVVNHIIDSALLGCDFPSPHYLLSGRYSGCEKYREFVDRL | |
| | KDSFVAVDDSGMPICIPIRSETQFFQEQIDKRLPGYSKIDMS | |
| | KMNWNSKRKIVSHYQPQRKVGYKLVAARDSIESIKDRLLD | |
| | LNKKQGTDTVIRDATIESIRDEKLKSRMLNNPDRAAYYLIE | |
| | YVQQSIKYGSNEEVYHPTAIEMKQMRDEFVNDDPELYVTE | |
| | DKELRKQVPNIFGMRIQTGQSESQISFHRVCTQGDTVHHHH | |
| | YMQDIGIRHIAIVVHIDKKGKKQNQAITVTRQGKVYIGLGT | |
| | ARKSVDRSFGMTLQGRAFGEKGSRKKWDRQWHREFRAFC | |
| | KATWNVERIHILTAGCVFIDTDGTTYFVRNMDSQDKGIRIA | |
| | TAKRKFVEIERVFKSPHAYQKYLSKMNAITKSGGSSGGSSG | |
| | SETPGTSESATPESSGGSSGGSSKRPAATKKAGQAKKKK | |

TABLE 10
Target and RNA Guide Sequences

| Guide | PAM | Target Sequence | RNA Guide Sequence* |
|---|---|---|---|
| AAVS1-T6 | AACC | GGATCCCTAG | GGAUCCCUAGCUGGGCCCUGguuguauucggaugggagua |
| 6 | | CTGGGCCCTG | aaagguugaguacaccuggaaacaaaacgacauaaagguguucucccaucu |
| | | (SEQ ID NO: 51) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 52) |
| AAVS1-T7 | AACC | GGCTATAATC | GGCUAUAAUCCAUGCUGACCguuguauucggaugggagua |
| | | CATGCTGACC | aaagguugaguacaccuggaaacaaaacgacauaaagguguucucccaucu |
| | | (SEQ ID NO: 53) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 54) |
| EMX1-T4 | AGCC | ATGACTCCAG | AUGACUCCAGGCCUCCCCAAguuguauucggaugggagua |
| | | GCCTCCCCAA | aaagguugaguacaccuggaaacaaaacgacauaaagguguucucccaucu |
| | | (SEQ ID NO: 55) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 56) |
| EMX1-T6 | AGCC | TCCAAAGCTT | UCCAAAGCUUUCCUUCUUUGguuguauucggaugggagua |
| | | TCCTTCTTTG | aaagguugaguacaccuggaaacaaaacgacauaaagguguucucccaucu |
| | | (SEQ ID NO: 57) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 58) |
| VEGFA-T2 | AACC | CCTCCACCAG | CCUCCACCAGUCAUGGUGACguuguauucggaugggagua |
| | | TCATGGTGAC | aaagguugaguacaccuggaaacaaaacgacauaaagguguucucccaucu |
| | | (SEQ ID NO: 59) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 60) |
| VEGFA-T4 | AGCC | CTCTACACAA | CUCUACACAAAUCUCUCCAGguuguauucggaugggagua |
| | | ATCTCTCCAG | aaagguugaguacaccuggaaacaaaacgacauaaagguguucucccaucu |
| | | (SEQ ID NO: 61) | guguacaaagagcagauauaugaguugauaaguaauauguugcacguaug |
| | | | gacuuugacagucuguaccucugcgugaaagacauauugcguuauucaua |
| | | | uaucugugagucuguucuaaauuuaaagguuuagggaa |
| | | | (SEQ ID NO: 62) |

*Spacer in upper case and scaffold (SEQ ID NO: 63) in lower case

HEK293T cells were transfected with plasmids expressing the Nuclease S polypeptides and the gRNAs provided herein following the methods provided in Example 1 above. Gene editing efficiencies were determined by NGS following the methods also provided in Example 1. The NGS results were analyzed also following the methods provided in Example 1 above.

For each target, indel ratios, referring to the percentage of NGS reads comprising indels, were calculated for each sample and its cognate no protein control. Targets comprising a higher percentage of indels when Nuclease S was included in the transfection were indicative of DNA editing outcomes in the cell.

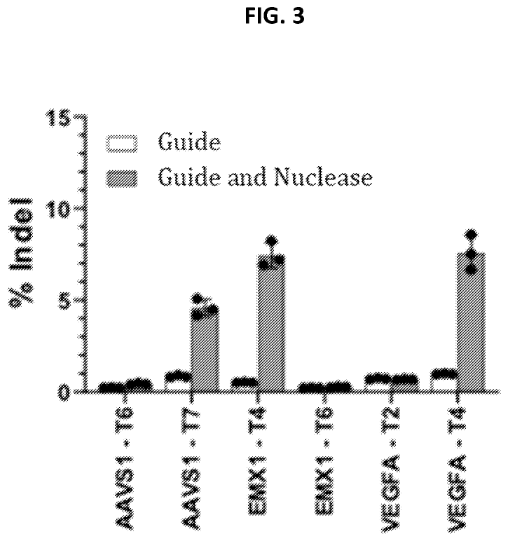

As shown in FIG. 3, three of the six targets tested demonstrated a greater level of indels observed when the Nuclease S (SEQ ID NO: 49) plasmid was present.

### Example 6: Effectiveness Variant CRISPR Nucleases S for Targeting of Mammalian Genes

This Example describes indel assessment on mammalian targets using variants of CRISPR Nuclease S transfected into HEK293T cells following the methods disclosed in Example 5 above.

Arginine scanning mutagenesis was performed to individually substitute each non-arginine residue of the reference CRISPR nuclease (Nuclease S; SEQ ID NO: 49) to arginine. SEQ ID NO: 49 is referred to herein as the reference sequence. This resulted in 916 single arginine substitution variants. Nucleic acids encoding the reference and each Nuclease S variant were then individually cloned into a pcDNA3.1 backbone (Invitrogen), and the plasmids were maxi-prepped and diluted. The plasmids comprised a CMV promoter, a first NLS (KRTADGSEFESPKKKRKV; SEQ ID NO: 3) upstream of the coding sequence, and an XTEN linker (SGGSSGGSSGSETPGTSESATPESSGGSSGGSS; SEQ ID NO: 5) followed by a second NLS (KRPAATKKAGQAKKKK; SEQ ID NO: 4) downstream of the coding sequence. See also Example 5 above.

The RNA guide and target sequences are shown in Table 11. RNA guides were cloned into a pUC19 backbone (New England Biolabs®) following the U6 PolIII promoter and terminated with a 6×polyT sequence. The U6 PolIII uses a +1 G at the start of the transcript (i.e., the 5′ end of the RNA) for more efficient transcription that is excluded from the sequences described in Table 11. The plasmids were purified using a maxi-prep kit and diluted.

TABLE 11
Mammalian Targets and Corresponding RNA guides*

| Target | | PAM | Target | RNA guide |
|---|---|---|---|---|
| identifier | Guide | Sequence | sequence | sequence |
| AAVS1 | AAVS1-T7 | AACC | SEQ ID NO: 53 | SEQ ID NO: 54 |
| EMX1 | EMX1-T4 | AGCC | SEQ ID NO: 55 | SEQ ID NO: 56 |
| VEGFA | VEGFA-T4 | AGCC | SEQ ID NO: 61 | SEQ ID NO: 62 |

*See Table 10 above for sequence details

For each target, indel ratios, referring to the percentage of NGS reads comprising indels, were calculated for the reference and for each variant. The indel ratios used for fold change calculations were the average of two technical replicates. To then calculate fold change in indel ratios at a particular target, the indel ratio for each variant was divided by the indel ratio for the reference. Table 12 shows fold change in indel ratios for each target and as an average of all targets. Numbering is relative to the reference nuclease of SEQ ID NO: 49 (i.e., without an NLS). For example, at the AAVS1 target, the indel ratio for the D844R variant was 6.37 times that of the reference indel ratio; at the EMX1 target, the indel ratio for the D844R variant was 5.15 times that of the reference indel ratio; and at the VEGFA target, the indel ratio for the D844R variant was 5.51 times that of the reference indel ratio. The average fold change in indel ratio for the D844R variant at all targets was 5.27 times that of the indel ratio for the reference at all targets.

As shown in Table 12, 29 variants with single arginine substitutions (left column) were characterized as yielding at least a 1.5× increase in indel ratio relative to the reference indel ratio, when averaged across the three targets (right column).

TABLE 12
Fold Change in Indel Ratios*

| | | | | AAVS1, EMX1, and |
|---|---|---|---|---|
| | | | | VEGFA average fold |
| Substitution | AAVS1 | EMX1 | VEGFA | change |
| D844R | 6.37 | 5.15 | 5.51 | 5.27 |
| D179R | 6.38 | 4.46 | 5.23 | 4.72 |
| H280R | 5.45 | 4.23 | 3.05 | 3.84 |
| W509R | 2.92 | 3.24 | 3.79 | 3.43 |
| I322R | 2.52 | 2.34 | 3.60 | 2.76 |
| V324R | 1.88 | 2.06 | 3.46 | 2.53 |
| T72R | 4.63 | 3.05 | 1.37 | 2.49 |
| N76R | 3.27 | 2.61 | 2.07 | 2.43 |
| I845R | 2.62 | 2.19 | 2.09 | 2.15 |
| Q200R | 2.74 | 2.14 | 2.01 | 2.10 |
| E277R | 2.40 | 2.11 | 1.92 | 2.05 |
| V504R | 1.92 | 1.97 | 2.06 | 2.00 |
| A65R | 2.74 | 2.13 | 1.62 | 1.96 |
| E334R | 1.48 | 1.63 | 2.45 | 1.90 |
| A336R | 1.55 | 1.71 | 2.24 | 1.89 |
| G298R | 1.96 | 1.62 | 2.18 | 1.81 |
| L316R | 1.42 | 1.57 | 2.29 | 1.81 |
| Y281R | 1.98 | 1.82 | 1.76 | 1.80 |
| I62R | 2.76 | 1.98 | 1.44 | 1.80 |
| E199R | 1.96 | 1.59 | 1.94 | 1.70 |
| G71R | 1.96 | 1.73 | 1.52 | 1.66 |
| L294R | 2.36 | 1.86 | 1.08 | 1.60 |
| I328R | 1.02 | 1.27 | 2.23 | 1.59 |
| L68R | 2.27 | 1.85 | 1.02 | 1.57 |
| C13R | 2.21 | 1.77 | 1.10 | 1.55 |
| S67R | 2.20 | 1.71 | 1.21 | 1.55 |
| L879R | 1.64 | 1.39 | 1.83 | 1.53 |
| V698R | 0.92 | 1.53 | 1.49 | 1.52 |
| N83R | 1.85 | 1.63 | 1.27 | 1.51 |

*Variant indel ratio/Reference indel ratio

241 variants were analyzed as having indel ratios 1×-1.5× of the reference indel ratios, when averaged across the three targets: E273R, P15R, D707R, I10R, Q783R, S190R, S297R, W180R, T197R, E247R, L7R, I688R, Y312R, F210R, A64R, K858R, F575R, T303R, Y983R, S296R, E117R, T299R, T75R, I293R, Q43R, I929R, L340R, N14R, K315R, D97R, S516R, E8R, P323R, D800R, S711R, N237R, D61R, D495R, I265R, N40R, L6R, D2R, Y203R, L251R, Q208R, I856R, D332R, N809R, P326R, T302R, G943R, E122R, E802R, K58R, T941R, S557R, E242R, T727R, K782R, V187R, E969R, 939R, S310R, E799R, S120R, L255R, K29R, I568R, K9R, P649R, K956R, T835R, S21R, Q545R, S379R, E927R, E4R, D12R, Q723R, E60R, F967R, P506R, A732R, Y5R, V321R, L36R, K295R, T207R, A555R, E740R, G361R, A88R, H855R, E181R, E118R, S377R, G319R, H930R, E248R, Q307R, I209R, F304R, A121R, V48R, W205R, D942R, K861R, I46R, E17R, Q23R, Q243R, E131R, A111R, Y285R, K38R, N749R, P378R, S309R, G724R, K966R, L219R, D113R, S708R, E116R, M414R, P456R, T725R, N184R, N864R, D714R, S540R, C369R, K288R, Q763R, P572R, Y239R, V42R, E772R, V728R, A582R, D406R, F19R, E196R, Y329R, H274R, I508R, A314R, E794R, D123R, K228R, E220R, S240R, L39R, K403R, D366R, F890R, I241R, Y216R, T944R, K801R, I498R, D94R, K862R, S684R, L984R, N790R, E222R, T562R, G483R, L233R, G860R, A28R, K593R, D786R, L636R, G182R, I194R, E971R, A989R, A882R, K594R, C226R, V313R, K272R, Q114R, K104R, V633R, K261R, T798R, A979R, N221R, V968R, L795R, I362R, Q289R, N174R, E735R, D234R, N988R, K238R, K385R, K986R, A189R, N446R, Y235R, K859R, F578R, D792R, T519R, K398R, K805R, Y946R, L354R, L160R, N925R, T488R, S442R, N224R, I990R, A89R, G41R, F195R, K80R, Y443R, T485R, V227R, A922R, Q393R, K159R, K267R, D644R, L803R, N231R, Q806R, K992R, Q662R, S212R, Q411R, K500R, M987R, and A490R.

The remaining variants (646 variants) resulted in decreased indel ratios relative to the reference indel ratios (fold change in indel ratios of less than 1.0): P613R, D388R, S387R, K392R, T991R, D264R, K98R, I877R, N560R, K430R, L669R, D115R, K489R, Q150R, K364R, H269R, Q832R, V217R, S215R, K921R, T544R, K44R, K249R, P451R, Q254R, N257R, N292R, N595R, K964R, K501R, K667R, Q410R, E258R, Q185R, I652R, D246R, G563R, P615R, S889R, D16R, P670R, D418R, L202R, K400R, P146R, K569R, K574R, T363R, K85R, L350R, L425R, K766R, S904R, C920R, T399R, A963R, D586R, S656R, N581R, I675R, I317R, I360R, T359R, Y980R, N720R, I650R, T119R, K391R, T244R, A642R, S232R, D666R, S268R, F112R, Q250R, T440R, Y480R, K679R, P750R, E710R, K155R, Y767R, Q954R, H520R, I564R, K389R, Q384R, F612R, D726R, K628R, T556R, L431R, S367R, D252R, V926R, K469R, H913R, Y229R, K502R, A390R, Q402R, K214R, D634R, L439R, K339R, A270R, A163R, Q865R, V395R, E468R, I18R, S646R, K697R, T20R, D611R, K253R, E759R, Q664R, K721R, K499R, I779R, M311R, N143R, S985R, A381R, P45R, N770R, Y579R, S376R, K741R, K331R, K975R, G335R, G596R, S109R, K674R, I463R, S396R, A918R, K358R, T923R, A193R, N260R, Q659R, K138R, K982R, K542R, L491R, S744R, E144R, Q762R, N513R, E320R, T962R, Y286R, K284R, S405R, D791R, G647R, G300R, K548R, K171R, S559R, F640R, G671R, K325R, A526R, C404R, K637R, H616R, M784R, E158R, K907R, K79R, T870R, K701R, Q693R, D718R, G625R, S673R, S271R, K902R, F503R, K136R, K380R, E787R, P653R, D834R, S213R, E631R, V599R, Y275R, I960R, Q338R, S374R, S736R, M892R, T156R, E346R, S614R, K383R, S372R, L230R, D940R, G95R, Q521R, Y154R, V510R, S639R, K177R, K687R, A851R, S453R, L553R, H308R, K382R, K124R, I56R, Y577R, T522R, D492R, K416R, T283R, K685R, S620R, A318R, E407R, L53R, K401R, Q567R, G441R, V370R, F974R, M648R, D135R, D638R, Y774R, A476R, P188R, K906R, G818R, Y755R, D147R, C626R, H691R, N256R, Y590R, T54R, S176R, D645R, V854R, A561R, G524R, A96R, L330R, F632R, V583R, Q863R, P438R, S487R, K874R, L747R, V37R, Q911R, I333R, E657R, Y692R, N192R, E225R, K344R, N523R, E327R, G541R, T126R, I654R, P204R, W924R, A291R, E148R, K167R, L100R, E481R, D458R, D751R, A426R, S769R, D731R, K511R, I729R, G621R, N186R, S885R, P585R, Y342R, L702R, M484R, G375R, H92R, K394R, H849R, G873R, N397R, S279R, D857R, C651R, N748R, F409R, S419R, E591R, A55R, K884R, A373R, E627R, Y348R, D245R, L223R, Q149R, K743R, E437R, Y617R, S570R, T933R, N588R, I444R, Y700R, Y629R, L259R, F278R, E445R, I815R, E164R, V368R, L618R, K421R, T881R, A482R, D151R, Y756R, A551R, D525R, Y110R, F152R, K722R, S129R, K536R, K128R, I49R, Q816R, Q695R, V108R, Y672R, I571R, I262R, G436R, I852R, T893R, L198R, L619R, S624R, H351R, K713R, N162R, D592R, A704R, D429R, G475R, A337R, P355R, V139R, F415R, M781R, E408R, I665R, I931R, I59R, T478R, N681R, G833R, V836R, Y623R, L717R, A705R, E529R, S822R, S953R, Y102R, F538R, C936R, A934R, V353R, Y30R, G27R, E771R, I539R, V52R, N69R, P566R, P573R, E901R, G597R, V140R, G609R, P175R, T945R, Q895R, V773R, T356R, D51R, V789R, G878R, D422R, H978R, N172R, F532R, E780R, G903R, I486R, F494R, F417R, L584R, L719R, D887R, P808R, V63R, P465R, L537R, V598R, K474R, L86R, G91R, A101R, V973R, I170R, S678R, T546R, F660R, W282R, H827R, A866R, Q981R, G880R, V643R, A343R, N471R, C467R, L558R, F461R, N462R, D676R, L290R, D955R, L206R, P57R, F428R, C103R, E663R, T868R, G412R, L345R, P793R, G517R, T341R, S78R, C168R, C178R, P413R, C157R, C305R, P479R, F587R, L161R, V576R, A493R, I734R, T658R, C432R, P694R, V948R, F455R, C183R, P547R, L533R, V689R, Y434R, A778R, S459R, C610R, K77R, E132R, V869R, E371R, C33R, C470R, E527R, Y460R, W908R, M423R, F22R, L464R, I824R, H448R, A50R, W682R, A433R, N683R, V797R, S352R, D604R, E447R, S690R, V641R, I554R, F916R, V761R, E165R, I810R, T34R, T26R, Y497R, Q823R, I145R, G891R, L894R, V141R, K589R, H775R, P776R, L757R, L450R, E427R, S820R, A753R, A898R, Y457R, P977R, S507R, I449R, L514R, I970R, I603R, Y796R, E915R, I93R, M680R, G32R, C435R, M951R, M746R, D909R, L515R, L608R, L349R, V829R, A606R, L424R, G107R, I709R, I365R, A754R, T733R, I850R, H543R, L742R, M518R, H505R, I602R, L607R, D739R, I765R, T777R, V35R, Y876R, V703R, G846R, N950R, L137R, S347R, G768R, V266R, I24R, M47R, D952R, I847R, G935R, L134R, C534R, F661R, S976R, H837R, I712R, L357R, M677R, H838R, L716R, A961R, L530R, T201R, V937R, L550R, I958R, F947R, I867R, G957R, T831R, D25R, C830R, V153R, H601R, N600R, A466R, Q872R, L932R, G699R, T817R, Y760R, I758R, V853R, Y31R, S605R, S764R, I737R, W912R, F899R, V99R, H839R, V807R, Y841R, Q843R, E821R, M842R, F919R, M813R, I90R, F811R, F788R, G900R, V886R, H840R, V133R, S825R, F938R, Q819R, G812R, G896R, V875R, and F826R.

Based on this experiment, the following variants of Nuclease S of SEQ ID NO: 49 were selected for further engineering: D844R, D179R, H280R, W509R, I322R, T72R, N76R, and I845R.

### Example 7: Effectiveness of Combination CRISPR Nuclease Variants for Targeting of Mammalian Genes

This Example describes indel assessment on mammalian targets using CRISPR nuclease variants derived from Nuclease S (SEQ ID NO: 49), comprising two or more substitutions identified as increasing indel activity in Example 6. 90 Nuclease S variants with combinations of arginine substitutions were tested.

Each Nuclease S variant was cloned as described in Example 6. HEK293T cells were further transfected with a plasmid encoding a Nuclease S variant and a plasmid encoding an RNA guide of Table 11, followed by NGS analysis, as described in Example 5. For each target, indel ratios, referring to the percentage of NGS reads comprising indels, were calculated for the reference Nuclease S (SEQ ID NO: 49) and for each Nuclease S variant. The indel ratios shown in Table 13 were calculated as the average of two bioreplicates, each of which contained two technical replicates.

TABLE 13
Indel Ratios for Mammalian Targets

| | AAVS1 | EMX1 | VEGFA | Average |
|---|---|---|---|---|
| | average | average | average | indel |
| | indel | indel | indel | ratio |
| Substitutions | ratio | ratio | ratio | targets |
| H280R, T72R, I845R | 0.3327 | 0.2915 | 0.2700 | 0.2981 |
| W509R, T72R, I845R | 0.3052 | 0.3053 | 0.2811 | 0.2972 |
| W509R, I845R | 0.2470 | 0.2936 | 0.2790 | 0.2732 |
| H280R, W509R, T72R, I845R | 0.3026 | 0.2567 | 0.2559 | 0.2718 |
| D844R, W509R | 0.2649 | 0.2757 | 0.2671 | 0.2693 |
| W509R, T72R | 0.2901 | 0.2766 | 0.2368 | 0.2678 |
| D844R, W509R, I845R | 0.2908 | 0.2394 | 0.2570 | 0.2624 |
| H280R, W509R, I845R | 0.2638 | 0.2398 | 0.2639 | 0.2558 |
| D844R, H280R | 0.2632 | 0.2532 | 0.2489 | 0.2551 |
| D179R, T72R, I845R | 0.2671 | 0.2213 | 0.2517 | 0.2467 |
| D844R, W509R, T72R | 0.2167 | 0.2522 | 0.2660 | 0.2450 |
| H280R, T72R | 0.2767 | 0.2555 | 0.1935 | 0.2419 |
| D844R, H280R, I845R | 0.2817 | 0.2131 | 0.2290 | 0.2413 |
| D179R, T72R | 0.2481 | 0.2119 | 0.2612 | 0.2404 |
| H280R, W509R, T72R | 0.2780 | 0.2150 | 0.2202 | 0.2377 |
| H280R, I845R | 0.2168 | 0.2584 | 0.2335 | 0.2362 |
| D844R, H280R, W509R | 0.2417 | 0.2190 | 0.2372 | 0.2326 |
| D844R, H280R, W509R, I845R | 0.2708 | 0.1792 | 0.2348 | 0.2283 |
| D844R, I322R | 0.2360 | 0.2027 | 0.2362 | 0.2250 |
| D844R, D179R | 0.2581 | 0.1920 | 0.2248 | 0.2250 |
| D844R, H280R, T72R | 0.1987 | 0.2310 | 0.2438 | 0.2245 |
| D844R, H280R, W509R, T72R | 0.1890 | 0.2074 | 0.2599 | 0.2188 |
| D179R, I845R | 0.2394 | 0.1880 | 0.2259 | 0.2177 |
| I322R, T72R, I845R | 0.2091 | 0.1923 | 0.2413 | 0.2142 |
| D179R, H280R, T72R, I845R | 0.2291 | 0.1429 | 0.2423 | 0.2048 |
| D179R, H280R, T72R | 0.2249 | 0.1365 | 0.2410 | 0.2008 |
| D844R, I322R, I845R | 0.2400 | 0.1468 | 0.2151 | 0.2006 |
| D844R, D179R, I845R | 0.2420 | 0.1424 | 0.2061 | 0.1968 |
| D844R, I322R, T72R | 0.1750 | 0.1765 | 0.2296 | 0.1937 |
| D844R, W509R, T72R, I845R | 0.1583 | 0.1785 | 0.2401 | 0.1923 |
| D179R, H280R, I845R | 0.2146 | 0.1248 | 0.2360 | 0.1918 |
| D844R, D179R, H280R | 0.2069 | 0.1249 | 0.2245 | 0.1854 |
| D844R, D179R, T72R | 0.1695 | 0.1765 | 0.2096 | 0.1852 |
| D179R, W509R, T72R | 0.2052 | 0.1338 | 0.2139 | 0.1843 |
| D179R, H280R, W509R, T72R | 0.1996 | 0.1300 | 0.2150 | 0.1816 |
| I322R, I845R | 0.1637 | 0.1529 | 0.2263 | 0.1810 |
| D179R, W509R, T72R, I845R | 0.1978 | 0.1316 | 0.2018 | 0.1771 |
| D179R, W509R, I845R | 0.1998 | 0.1205 | 0.2108 | 0.1770 |
| D844R, D179R, W509R | 0.1992 | 0.1195 | 0.2036 | 0.1741 |
| I322R, T72R | 0.1661 | 0.1510 | 0.2014 | 0.1729 |
| D179R, H280R, W509R, I845R | 0.1799 | 0.1146 | 0.2116 | 0.1687 |
| D844R, I845R | 0.1776 | 0.1067 | 0.1952 | 0.1598 |
| D844R, H280R, T72R, I845R | 0.1323 | 0.1520 | 0.1844 | 0.1562 |
| D844R, D179R, H280R, W509R | 0.1672 | 0.1051 | 0.1886 | 0.1536 |
| H280R, W509R | 0.0985 | 0.1784 | 0.1789 | 0.1519 |
| H280R, I322R, T72R, I845R | 0.1854 | 0.0867 | 0.1808 | 0.1510 |
| D844R, D179R, W509R, T72R | 0.1201 | 0.1227 | 0.2004 | 0.1477 |
| D844R, D179R, H280R, T72R | 0.1181 | 0.1108 | 0.2077 | 0.1455 |
| H280R, I322R, T72R | 0.1804 | 0.0755 | 0.1750 | 0.1436 |
| D844R, H280R, I322R | 0.1594 | 0.0745 | 0.1908 | 0.1416 |
| D844R, I322R, T72R, I845R | 0.1098 | 0.1116 | 0.2000 | 0.1405 |
| D179R, H280R | 0.1237 | 0.0968 | 0.1973 | 0.1392 |
| D844R, D179R, T72R, I845R | 0.1178 | 0.1104 | 0.1818 | 0.1367 |
| W509R, I322R, T72R | 0.1545 | 0.0771 | 0.1606 | 0.1307 |
| H280R, I322R, I845R | 0.1596 | 0.0634 | 0.1685 | 0.1305 |
| D844R, T72R | 0.0734 | 0.1166 | 0.1929 | 0.1276 |
| D179R, W509R | 0.1053 | 0.0977 | 0.1707 | 0.1246 |
| D179R, H280R, W509R | 0.1114 | 0.0917 | 0.1668 | 0.1233 |
| D844R, D179R, H280R, I845R | 0.1472 | 0.0680 | 0.1484 | 0.1212 |
| D179R, I322R, T72R | 0.1388 | 0.0563 | 0.1643 | 0.1198 |
| D844R, D179R, W509R, I845R | 0.1474 | 0.0768 | 0.1349 | 0.1197 |
| D179R, I322R, T72R, I845R | 0.1375 | 0.0634 | 0.1573 | 0.1194 |
| D844R, H280R, I322R, T72R | 0.1037 | 0.0656 | 0.1731 | 0.1141 |
| D844R, W509R, I322R | 0.1260 | 0.0651 | 0.1512 | 0.1141 |
| H280R, W509R, I322R, T72R | 0.1435 | 0.0624 | 0.1334 | 0.1131 |
| W509R, I322R, I845R | 0.1324 | 0.0583 | 0.1440 | 0.1116 |
| D179R, I322R, I845R | 0.1212 | 0.0499 | 0.1436 | 0.1049 |
| D179R, H280R, I322R, T72R | 0.1065 | 0.0499 | 0.1550 | 0.1038 |
| D844R, D179R, I322R | 0.1176 | 0.0519 | 0.1306 | 0.1000 |
| D844R, T72R, I845R | 0.0519 | 0.0767 | 0.1677 | 0.0988 |
| D844R, W509R, I322R, T72R | 0.0842 | 0.0597 | 0.1460 | 0.0966 |
| D844R, H280R, W509R, I322R | 0.1122 | 0.0487 | 0.1206 | 0.0938 |
| D844R, H280R, I322R, I845R | 0.1200 | 0.0413 | 0.1186 | 0.0933 |
| D844R, D179R, I322R, T72R | 0.0755 | 0.0527 | 0.1433 | 0.0905 |
| D179R, W509R, I322R, T72R | 0.1048 | 0.0485 | 0.1182 | 0.0905 |
| H280R, W509R, I322R, I845R | 0.1052 | 0.0488 | 0.1112 | 0.0884 |
| T72R, I845R | 0.0832 | 0.0784 | 0.0959 | 0.0858 |
| D179R, H280R, I322R, I845R | 0.0951 | 0.0403 | 0.1158 | 0.0837 |
| H280R, I322R | 0.0774 | 0.0466 | 0.1185 | 0.0808 |
| D844R, D179R, H280R, I322R | 0.0851 | 0.0407 | 0.1120 | 0.0793 |
| D844R, W509R, I322R, I845R | 0.1056 | 0.0363 | 0.0920 | 0.0780 |
| W509R, I322R | 0.0699 | 0.0494 | 0.1083 | 0.0759 |
| D844R, D179R, W509R, I322R | 0.0806 | 0.0407 | 0.0946 | 0.0720 |
| D179R, W509R, I322R, I845R | 0.0861 | 0.0389 | 0.0891 | 0.0714 |
| D179R, I322R | 0.0574 | 0.0345 | 0.1115 | 0.0678 |
| D179R, H280R, I322R | 0.0592 | 0.0329 | 0.1075 | 0.0665 |
| D844R, D179R, I322R, I845R | 0.0795 | 0.0299 | 0.0805 | 0.0633 |
| D179R, H280R, W509R, I322R | 0.0507 | 0.0328 | 0.0934 | 0.0590 |
| H280R, W509R, I322R | 0.0559 | 0.0346 | 0.0818 | 0.0574 |
| D179R, W509R, I322R | 0.0426 | 0.0335 | 0.0858 | 0.0540 |
| Reference | 0.0048 | 0.0265 | 0.0333 | 0.0215 |

As shown in Table 13, each of the Nuclease S variants with combinations of amino acid substitutions exhibited higher indel activity than the reference CRISPR nuclease (SEQ ID NO: 49). 9 Nuclease S variants resulted in indel ratios of over 0.25 when averaged across the three targets, indicating that over 25% of NGS reads comprised indels. These 9 Nuclease S variants comprised the following substitution combinations: a) H280R, T72R, I845R; b) W509R, T72R, I845R; c) W509R, I845R; d) H280R, W509R, T72R, I845R; e) D844R, W509R; f) W509R, T72R; g) D844R, W509R, I845R; h) H280R, W509R, I845R; and i) D844R, H280R. 18 Nuclease S variants resulted in indel ratios between 0.2 and 0.24 when averaged across the three targets, indicating between 20% and 24% of NGS reads comprised indels. 42 Nuclease S variants resulted in indel ratios of 0.1 to 0.19 when averaged across the three targets, indicating between 10% and 19% of NGS reads comprised indels. 21 Nuclease S variants resulted in indel ratios of 0.05 to 0.09 when averaged across the three targets, indicating between 5% and 9% of NGS reads comprised indels.

Based on this experiment, two Nuclease S variants comprising the following substitutions were selected for further testing: a) H280R, T72R, I845R and b) W509R, T72R, I845R. These two Nuclease S variants exhibited a 28-fold and 26-fold increase in indel activity compared to the reference Nuclease S (SEQ ID NO: 49), respectively.

### Example 8: Engineering and Effectiveness of Nickase Variants of Nuclease S for Targeting Mammalian Genes

This Example describes introducing mutations into the reference Nuclease S of SEQ ID NO: 49 that disrupt either the HNH or RuvC domains to produce a functional nickase. E447, H448, and N471 were identified as putative catalytic residues of the HNH domain of Nuclease S. D25, E371, and D604 positions were identified as putative catalytic residues of the RuvC domain of Nuclease S. These positions were identified by analyzing models generated with AlphaFold2 (Jumper et al., Nature 596: 583-9 (2021)) for structural regions resembling known HNH and RuvC active sites and/or by performing sequence alignments to other nucleases for which candidate positions had been previously identified. Examples of reference structures used to identify the HNH and RuvC active sites are represented with the following PDB IDs: 5h0m, 7eu9, 61tu, 7odf, 7lys, 8dc2, 4cmp, 4oo8, 7z4j, 5axw, 5b2o, 6kc8, 7utn, 8csz, 8ctl, 8dmb.

The coding sequence of Nuclease S was converted into an E. coli-codon optimized DNA sequence, synthesized, and cloned into a pET-28a(+) vector (Novagen), containing lac and T RNA polymerase promoters for gene expression. To test for nickase activity, individual alanine mutants were cloned for each of the positions identified as putative active site residues of the HNH and RuvC domains of Nuclease S. A leucine mutant was also cloned for position H448. Research grade plasmids were received from GenScript. The engineered nickase sequences are shown in Table 19. The codon encoding the substituted residue is capitalized, bold, and underlined in the nucleotide sequence, and the substituted residue is shown in bold and underlined in the amino acid sequence. The putative HNH-knockout nickases were anticipated to cleave the non-target strand but not the target strand. The putative RuvC-knockout nickases were anticipated to cleave the target strand but not the non-target strand.

TABLE 19
Sequences of Nuclease S and Nickase Variants Thereof

| Nuclease S | | |
|---|---|---|
| and Nickase | | |
| Variants | | |
| Thereof | Nucleotide sequence | Amino acid sequence |
| Nuclease S | atggatagggagtatctattagaaaaaatacgcgactgtaatccggatgaaa | SEQ ID NO: 49 |
| | tcttcactagcttccaaattgacaccggcgcgaagtactatggttgcaccgtg | (see Table 9 above) |
| | ttggttaaactaccgggggtacagaaaccgattatggtcatcgcggatgtgc | |
| | tgacggctattccaaagatcgaagacattgttgccgctcgttcgctgaatcgc | |
| | ggtacacgtcgcaccaacaagtcgaagaagcgtcgcaaccgcaagctgc | |
| | gtgcggcgattggtcatatcgacggcgcagataaagttttggcgtattgtaaa | |
| | cgtcgtggcgtgtcctatgctttcgaccaagacgaagaagaaacctcagct | |
| | gaagataagcgcacccgtaagtcgcgtgaggaggttctggacaaattgaa | |
| | ggtggttgttcgaaatgagatcccggatgagcagcaggacttcgtgtacaa | |
| | gacgtgcgagaaactgctgaacgcggaagagcggaaatgccgtatcaaa | |
| | aaccgtaacccgtcgaaatgcgactgggaaggttgcaatcaaaacgtgcc | |
| | ggcatcgcgaaacgcgatcttcgaaaccctggaacagaccttatatccgtg | |
| | gctgacgcagatttttcgcagctctaagtcctacgtacgtctggaaaacgag | |
| | cttaacgagtgcgttaaatacttaaacagcctggattatcgtaataaatacagt | |
| | atagagcagacggacgatgaggagaagcagctggataaacaattaaacaa | |
| | tgaactgaacaaaatccgcgacatcgtgaaatctcacgcatccaaagaaca | |
| | ctaccgcgaattcagtcactactggaccaagtattaccgtaaacaactagcta | |
| | atattttgaaatctagcggcaccggtcgtactacgttttgtcgtcaacattcctc | |
| | catgtatgtagcgaaacttattgccggagaagtgataccagtgaaacccgaa | |
| | atctatctgaaagatatcgagggtgcagcgcagaagttgacctatgcaaagt | |
| | tggagagttacctgctgcatagcgttctgccaaccctgaaaacgattggcat | |
| | caccaaaattgattccgtgtgtgttgaaagcgcgagcggtagctctccgagc | |
| | aaagccaaaaaacagaagcgttcagacaaggcgaagaagcaaaaagtga | |
| | gcaataaaaccaaaaaacagaagtgcagcgatgaggagttccaacaagg | |
| | cccgatgtttaagttcgacagccgcaaagacatgctgctggcggaattcgat | |
| | aagctgtgtgcatattgcggtgaaccgctcaccggtagctacattgagaacg | |
| | agcacatcctgccgcgtagtcggtttccgtatgatagctactttaatattctgc | |
| | cggcgtgtgagaagtgcaaccggcgaaagggcgcccgtaccccttatgaa | |
| | gcgggtatgaccattagcacgaaagcgctggatgcattcgatcgttatatca | |
| | agaagaaaaagttcgtgcacccgtccatctgggttaaacgtaacctgctgag | |
| | cggtatgacccatcagacgaacggcgacgccgaacgcgagctgcgttttct | |
| | gtgccgcaagctgttcatctccggcaaacacacccaaaccccgaaacgcct | |
| | ggctagactgatcgccaccagcctgagcaatgctaccggcatccgtccgc | |
| | agatcaagtctatcccgcctaagtttgtgtatttttaccgcaacgccgtgctgc | |
| | ccgactttaacaaatacgaagacaaaaagaacggtggtgtggtgaaccaca | |
| | ttattgactctgcgctgttaggctgcgattttccgtcgccgcactatctgttgtct | |
| | ggtcgttactccggttgtgagaagtaccgtgaattcgttgaccgcttgaagga | |
| | ttcctttgtcgcggtggacgacagcggtatgccgatttgcattccgattagaa | |
| | gcgaaacccaattttttcaagaacaaatcgacaagcgtttgccgggttactct | |
| | aaaattgacatgagcaagatgaactggaatagcaaacgcaagatcgttagc | |
| | cactaccaaccgcagcgtaaagttggttataaactggtcgcggcgcgtgac | |
| | tctatcgagtctatcaaggaccgtttgctggacctaaacaagaaacaaggca | |
| | cggatacggttattcgtgacgcgaccatcgagagcattcgtgatgaaaagct | |
| | caagtctcggatgctgaacaacccggaccgggctgcttactacttgattgag | |
| | tatgtgcagcaaagcatcaagtacgggtccaacgaagaggtttaccatccg | |
| | accgcaattgagatgaaacagatgcgtgacgagttcgtaaatgatgatcca | |
| | gaactgtacgtcaccgaggacaaggagttgagaaagcaggttccgaacatt | |
| | tttggcatgcgtatccagactggccagagcgaaagccagatctctttccacc | |
| | gtgtgtgcacccagggtgataccgttcatcaccaccattacatgcaggacat | |
| | cggcattcgtcatattgcgattgttgttcacattgacaaaaaaggtaaaaagc | |
| | agaaccaggcaatcactgtgacccgtcaaggtaaggtctacatcggcctgg | |
| | gtacggcgcgtaagagcgttgatcgctcctttggtatgaccctccagggcc | |
| | gtgctttcggtgaaaagggttcccgcaaaaagtgggatcgtcaatggcatc | |
| | gggagttccgtgcgttctgcaaggctacctggaatgtcgagcgtattcatatc | |
| | ctgaccgcaggctgcgtgttcatcgacactgatggcaccacctacttcgttc | |
| | gcaatatggatagccaggacaaaggcatccgcattgcgactgcgaaacgt | |
| | aaattcgtcgaaatcgagcgtgtttttaaaagcccgcacgcctatcagaaata | |
| | cctcagcaagatgaatgccatcaccaagv (SEQ ID NO: 78) | |

| | | |
|---|---|---|
| E447A (putative | atggatagggagtatctattagaaaaaatacgcgactgtaatccggatgaaa | MDREYLLEKIRDCNPDEIFTSFQI |
| HNH-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaagcgtcgcaaccgcaagctgc | DGADKVLAYCKRRGVSYAFDQD |
| | gtgcgcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVESA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | RKDMLLAEFDKLCAYCGEPLTGS |
| | atctatctgaaagatatcgagggtgcagcgcagaagttgacctatgcaaagt | YIENAHILPRSRFPYDSYFNILPAC |
| | tggagagttacctgctgcatagcgttctgccaaccctgaaaacgattggcat | EKCNRRKGARTPYEAGMTISTKA |
| | caccaaaattgattccgtgtgtgttgaaagcgcgagcggtagctctccgagc | LDAFDRYIKKKKFVHPSIWVKRN |
| | aaagccaaaaaacagaagcgttcagacaaggcgaagaagcaaaaagtga | LLSGMTHQTNGDAERELRFLCRK |
| | gcaataaaaccaaaaacagaagtgcagcgatgaggagttccaacaagg | LFISGKHTQTPKRLARLIATSLSN |
| | cccgatgtttaagttcgacagccgcaaagacatgctgctggcggaattcgat | ATGIRPQIKSIPPKFVYFYRNAVLP |
| | aagctgtgtgcatattgcggtgaaccgctcaccggtagctacattgagaac | DFNKYEDKKNGGVVNHIIDSALL |
| | GCGcacatcctgccgcgtagtcggtttccgtatgatagctactttaatattct | GCDFPSPHYLLSGRYSGCEKYRE |
| | gccggcgtgtgagaagtgcaaccggcgaaagggcgcccgtaccccttat | FVDRLKDSFVAVDDSGMPICIPIR |
| | gaagcgggtatgaccattagcacgaaagcgctggatgcattcgatcgttata | SETQFFQEQIDKRLPGYSKIDMSK |
| | tcaagaagaaaaagttcgtgcacccgtccatctgggttaaacgtaacctgct | MNWNSKRKIVSHYQPQRKVGYK |
| | gagcggtatgacccatcagacgaacggcgacgccgaacgcgagctgcgt | LVAARDSIESIKDRLLDLNKKQGT |
| | tttctgtgccgcaagctgttcatctccggcaaacacacccaaaccccgaaac | DTVIRDATIESIRDEKLKSRMLNN |
| | gcctggctagactgatcgccaccagcctgagcaatgctaccggcatccgtc | PDRAAYYLEIYVQQSIKYGSNEE |
| | cgcagatcaagtctatcccgcctaaagtttgtgtatttttaccgcaacgccgtgc | VYHPTAIEMKQMRDEFVNDDPEL |
| | tgcccgactttaacaaatacgaagacaaaaagaacggtggtgtggtgaacc | YVTEDKELRKQVPNIFGMRIQTG |
| | acattattgactctgcgctgttaggctgcgattttccgtcgccgcactatctgtt | QSESQISFHRVCTQGDTVHHHHY |
| | gtctggtcgttactccggttgtgagaagtaccgtgaattcgttgaccgcttga | MQDIGIRHIAIVVHIDKKGKKQN |
| | aggattcctttgtcgcggtggacgacagcggtatgccgatttgcattccgatt | QAITVTRQGKVYIGLGTARKSVD |
| | agaagcgaaacccaattttttcaagaacaaatcgacaagcgtttgccgggtt | RSFGMTLQGRAFGEKGSRKKWD |
| | actctaaaattgacatgagcaagatgaactggaatagcaaacgcaagatcg | RQWHREFRAFCKATWNVERIHIL |
| | ttagccactaccaaccgcagcgtaaagttggttataaactggtcgcggcgc | TAGCVFIDTDGTTYFVRNMDSQD |
| | gtgactctatcgagtctatcaaggaccgtttgctggacctaaacaagaaaca | KGIRIATAKRKFVEIERVFKSPHA |
| | aggcacggatacggttattcgtgacgcgaccatcgagagcattcgtgatga | YQKYLSKMNAITK (SEQ ID |
| | aaagctcaagtctcggatgctgaacaacccggaccgggctgcttactacttg | NO: 80) |
| | attgagtatgtgcagcaaagcatcaagtacgggtccaacgaagaggtttac | |
| | catccgaccgcaattgagatgaaacagatgcgtgacgagttcgtaaatgat | |
| | gatccagaactgtacgtcaccgaggacaaggagttgagaaagcaggttcc | |
| | gaacatttttggcatgcgtatccagactggccagagcgaaagccagatctct | |
| | ttccaccgtgtgtgcacccagggtgataccgttcatcaccaccattacatgca | |
| | ggacatcggcattcgtcatattgcgattgttgttcacattgacaaaaaaggtaa | |
| | aaagcagaaccaggcaatcactgtgacccgtcaaggtaaggtctacatcg | |
| | gcctgggtacggcgcgtaagagcgttgatcgctcctttggtatgaccctcca | |
| | gggccgtgctttcggtgaaaagggttcccgcaaaaagtgggatcgtcaatg | |
| | gcatcgggagttccgtgcgttctgcaaggctacctggaatgtcgagcgtatt | |
| | catatcctgaccgcaggctgcgtgttcatcgacactgatggcaccacctactt | |
| | cgttcgcaatatggatagccaggacaaaggcatccgcattgcgactgcgaa | |
| | acgtaaattcgtcgaaatcgagcgtgtttttaaaagcccgcacgcctatcaga | |
| | aatacctcagcaagatgaatgccatcaccaag (SEQ ID NO: 79) | |
| H448A (putative | atggatagggagtatctattagaaaaaatacgcgactgtaatccggatgaaa | MDREYLLEKIRDCNPDEIFTSFQI |
| HNH-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaagcgtcgcaaccgcaagctgc | DGADKVLAYCKRRGVSYAFDQD |
| | gtgcggcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQDFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVESA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | RKDMLLAEFDKLCAYCGEPLTGS |
| | atctatctgaaagatatcgagggtgcagcgcagaagttgacctatgcaaagt | YIENEAILPRSRFPYDSYFNILPAC |
| | tggagagttacttgctgcatagcgttctgccaaccctgaaaacgattggcat | EKCNRRKGARTPYEAGMTISTKA |
| | caccaaaattgattccgtgtgtgttgaaagcgcgagcggtagctctccgagc | LDAFDRYIKKKKFVHPSIWVKRN |
| | aaagccaaaaaacagaagcgttcagacaaggcgaagaagcaaaaagtga | LLSGMTHQTNGDAERELRFLCRK |
| | gcaataaaaccaaaaaacagaagtgcagcgatgaggagttccaacaagg | LFISGKHTQTPKRLARLIATSLSN |
| | cccgatgtttaagttcgacagccgcaaagacatgctgctggcggaattcgat | ATGIRPQIKSIPPKFVVFYRNAVLP |
| | aagctgtgtgcatattgcggtgaaccgctcaccggtagctacattgagaacg | DFNKYEDKKNGGVVNHIIDSALL |
| | agGCGatcctgccgcgtagtcggtttccgtatgatagctactttaatattct | GCDFPSPHYLLSGRYSGCEKYRE |
| | gccggcgtgtgagaagtgcaaccggcgaaagggcgcccgtaccccttat | FVDRLKDSFVAVDDSGMPICIPIR |
| | gaagcgggtatgaccattagcacgaaagcgctggatgcattcgatcgttata | SETQFFQEQIDKRLPGYSKIDMSK |
| | tcaagaagaaaaagttcgtgcacccgtccatctgggttaaacgtaacctgct | MNWNSKRKIVSHYQPQRKVGYK |
| | gagcggtatgacccatcagacgaacggcgacgccgaacgcgagctgctg | LVAARDSIESIKDRLLDLNKKQGT |
| | tttctgtgccgcaagctgttcatctccggcaaacacacccaaaccccgaaac | DTVIRDATIESIRDEKLKSRMLNN |
| | gcctggctagactgatcgccaccagcctgagcaatgctaccggcatccgtc | PDRAAYYLEIYVQQSIKYGSNEE |
| | cgcagatcaagtctatcccgcctaagtttgtgtatttttaccgcaacgccgtgc | VYHPTAIEMKQMRDEFVNDDPEL |
| | tgcccgactttaacaaatacgaagacaaaaagaacggtggtgtgtggtgaacc | YVTEDKELRKQVPNIFGMRIQTG |
| | acattattgactctgcgctgttaggctgcgattttccgtcgccgcactatctgtt | QSESQISFHRVCTQGDTVHHHHY |
| | gtctggtcgttactccggttgtgagaagtaccgtgaattcgttgaccgcttga | MQDIGIRHIAIVVHIDKKGKKQN |
| | aggattcctttgtcgcggtggacgacagcggtatgccgatttgcattccgatt | QAITVTRQGKVYIGLGTARKSVD |
| | agaagcgaaacccaattttttcaagaacaaatcgacaagcgtttgccgggtt | RSFGMTLQGRAFGEKGSRKKWD |
| | actctaaaattgacatgagcaagatgaactggaatagcaaacgcaagatcg | RQWHREFRAFCKATWNVERIHIL |
| | ttagccactaccaaccgcagcgtaaagttggttataaactggtcgcggcgc | TAGCVFIDTDGTTYFVRNMDSQD |
| | gtgactctatcgagtctatcaaggaccgtttgctggacctaaacaagaaaca | KGIRIATAKRKFVEIERVFKSPHA |
| | aggcacggatacggttattcgtgacgcgaccatcgagagcattcgtgatga | YQKYLSKMNAITK (SEQ ID |
| | aaagctcaagtctcggatgctgaacaacccggaccgggctgcttactacttg | NO: 82) |
| | attgagtatgtgcagcaaagcatcaagtacgggtccaacgaagaggtttac | |
| | catccgaccgcaattgagatgaaacagatgcgtgacgagttcgtaaatgat | |
| | gatccagaactgtacgtcaccgaggacaaggagttgagaaagcaggttcc | |
| | gaacatttttggcatgcgtatccagactggccagagcgaaagccagatctct | |
| | ttccaccgtgtgtgcacccagggtgataccgttcatcaccaccattacatgca | |
| | ggacatcggcattcgtcatattgcgattgttgttcacattgacaaaaaaggtaa | |
| | aaagcagaaccaggcaatcactgtgacccgtcaaggtaaggtctacatcg | |
| | gcctgggtacggcgcgtaagagcgttgatcgctcctttggtatgaccctcca | |
| | gggccgtgctttcggtgaaaagggttcccgcaaaaagtgggatcgtcaatg | |
| | gcatcgggagttccgtgcgttctgcaaggctacctggaatgtcgagcgtatt | |
| | catatcctgaccgcaggctgcgtgttcatcgacactgatggcaccacctactt | |
| | cgttcgcaatatggatagccaggacaaaggcatccgcattgcgactgcgaa | |
| | acgtaaattcgtcgaaatcgagcgtgtttttaaaagcccgcacgcctatcaga | |
| | aatacctcagcaagatgaatgccatcaccaag (SEQ ID NO: 81) | |
| H448L (putative | atggatagggagtatctattagaaaaaatacgcgactgtaatccggatgaaa | MDREYLLEKIRDCNPDEIFTSFQI |
| HNH-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaagcgtcgcaaccgcaagctgc | DGADKVLAYCKRRGVSYAFDQD |
| | gtgcggcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQDFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVESA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | RKDMLLAEFDKLCAYCGEPLTGS |
| | atctatctgaaagatatcgagggtgcagcgcagaagttgacctatgcaaagt | YIENELILPRSRFPYDSYFNIKPAC |
| | tggagagttacctgctgcatagcgttctgccaaccctgaaaacgattggcat | EKCNRRKGARTPYEAGMTISTKA |
| | caccaaaattgattccgtgtgtgttgaaagcgcgagcggtagctctccgagc | LDAFDRYIKKKKFVHPSIWVKRN |
| | aaagccaaaaaacagaagcgttcagacaaggcgaagaagcaaaaagtga | LLSGMTHQTNGDAERELRFLCRK |
| | gcaataaaaccaaaaaacagaagtgcagcgatgaggagttccaacaagg | LFISGKHTQTPKRLARLIATSLSN |
| | cccgatgtttaagttcgacagccgcaaagacatgctgctggcggaattcgat | ATGIRPQIKSIPPKFVYFYRNAVLP |
| | aagctgtgtgcatattgcggtgaaccgctcaccggtagctacattgagaacg | DFNKYEDKKNGGVVNHIIDSALL |
| | agCTGatcctgccgcgtagtcggtttccgtatgatagctactttaatattctg | GCDFPSPHYLLSGRYSGCEKYRE |
| | ccggcgtgtgagaagtgcaaccggcgaaagggcgcccgtaccccttatga | FVDRLKDSFVAVDDSGMPICIPIR |
| | agcgggtatgaccattagcacgaaagcgctggatgcattcgatcgttatatc | SETQFFQEQIDKRLPGYSKIDMSK |
| | aagaagaaaaagttcgtgcacccgtccatctgggttaaacgtaacctgctga | MNWNSKRKIVSHYQPQRKVGYK |
| | gcggtatgacccatcagacgaacggcgacgccgaacgcgagctgcgtttt | LVAARDSIESIKDRLLDLNKKQGT |
| | ctgtgccgcaagctgttcatctccggcaaacacacccaaaccccgaaacgc | DTVIRDATIESIRDEKLKSRMLNN |
| | ctggctagactgatcgccaccagcctgagcaatgctaccggcatccgtccg | PDRAAYYLIEYVQQSIKYGSNEE |
| | cagatcaagtctatcccgcctaagtttgtgtatttttaccgcaacgccgtgctg | VYHPTAIEMKQMRDEFVNDDPEL |
| | cccgactttaacaaatacgaagacaaaaagaacggtggtgtggtgaaccac | YVTEDKELRKQVPNIFGMRIQTG |
| | attattgactctgcgctgttaggctgcgattttccgtcgccgcactatctgttgt | QSESQISFHRVCTQGDTVHHHHY |
| | ctggtcgttactccggttgtgagaagtaccgtgaattcgttgaccgcttgaag | MQDIGIRHIAIVVHIDKKGKKQN |
| | gattcctttgtcgcggtggacgacagcggtatgccgatttgcattccgattag | QAITVTRQGKVYIGLGTARKSVD |
| | aagcgaaacccaattttttcaagaacaaatcgacaagcgtttgccgggttact | RSFGMTLQGRAFGEKGSRKKWD |
| | ctaaaattgacatgagcaagatgaactggaatagcaaacgcaagatcgtta | RQWHREFRAFCKATWNVERTHIL |
| | gccactaccaaccgcagcgtaaagttggttataaactggtcgcggcgcgtg | TAGCVFIDTDGTTYFVRNMDSQD |
| | actctatcgagtctatcaaggaccgtttgctggacctaaacaagaaacaagg | KGIRIATAKRKFVEIERVFKSPHA |
| | cacggatacggttattcgtgacgcgaccatcgagagcattcgtgatgaaaa | YQKYLSKMNAITK (SEQ ID |
| | gctcaagtctcggatgctgaacaacccggaccgggctgcttactacttgatt | NO: 84) |
| | gagtatgtgcagcaaagcatcaagtacgggtccaacgaagaggtttaccat | |
| | ccgaccgcaattgagatgaaacagatgcgtgacgagttcgtaaatgatgat | |
| | ccagaactgtacgtcaccgaggacaaggagttgagaaagcaggttccgaa | |
| | catttttggcatgcgtatccagactggccagagcgaaagccagatctctttcc | |
| | accgtgtgtgcacccagggtgataccgttcatcaccaccattacatgcagga | |
| | catcggcattcgtcatattgcgattgttgttcacattgacaaaaaaggtaaaaa | |
| | gcagaaccaggcaatcactgtgacccgtcaaggtaaggtctacatcggcct | |
| | gggtacggcgcgtaagagcgttgatcgctcctttggtatgaccctccaggg | |
| | ccgtgctttcggtgaaaagggttcccgcaaaaagtgggatcgtcaatggcat | |
| | cgggagttccgtgcgttctgcaaggctacctggaatgtcgagcgtattcatat | |
| | cctgaccgcaggctgcgtgttcatcgacactgatggcaccacctacttcgtt | |
| | cgcaatatggatagccaggacaaaggcatccgcattgcgactgcgaacg | |
| | taaattcgtcgaaatcgagcgtgtttttaaaagcccgcacgcctatcagaaat | |
| | acctcagcaagatgaatgccatcaccaag (SEQ ID NO: 83) | |
| N471A (putative | atggatagggagtatctattagaaaaaatacgcgactgtaatccggatgaaa | MDREYLLEKIRDCNPDEIFTSFQI |
| HNH-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaagcgtcgcaaccgcaagctgc | DGADKVLAYCKRRGVSYAFDQD |
| | gtgcggcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQDFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVESA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | TKDMLLAEFDKLCAYCGEPLTGS |
| | atctatctgaaagatatcgagggtgcagcgcagaagttgacctatgcaaagt | YIENEHILPRSRFPYDSYFNILPAC |
| | tggagagttacctgctgcatagcgttctgccaccctgaaaacgattggcat | EKCARRKGARTPYEAGMTISTKA |
| | caccaaaattgattccgtgtgtgttgaaagcgcgagcggtagctctccgagc | LDAFDRYIKKKKFVHPSIWVKRN |
| | aaagccaaaaaacagaagcgttcagacaaggcgaagaagcaaaaagtga | LLSGMTHQTNGDAERELRFLCRK |
| | gcaataaaaccaaaaaacagaagtgcagcgatgaggagttccaacaagg | LFISGKHTQTPKRLARLIATSLSN |
| | cccgatgtttaagttcgacagccgcaaagacatgctgctggcggaattcgat | ATGIRPQIKSIPPKFVYFYRNAVLP |
| | aagctgtgtgcatattgcggtgaaccgctcaccggtagctacattgagaacg | DFNKYEDKKNGGVVNHIIDSALL |
| | agcacatcctgccgcgtagtcggtttccgtatgatagctactttaatattctgc | GCDFPSPHYLLSGRYSGCEKYRE |
| | cggcgtgtgagaagtgcGCGcggcgaaagggcgcccgtaccccttat | FVDRLKDSFVAVDDSGMPICIPIR |
| | gaagcgggtatgaccattagcacgaaagcgctggatgcattcgatcgttata | SETQFFQEQIDKRLPGYSKIDMSK |
| | tcaagaagaaaaagttcgtgcacccgtccatctgggttaaacgtaacctgct | MNWNSKRKIVSHYQPQRKVGYK |
| | gagcggtatgacccatcagacgaacggcgacgccgaacgcgagctgcgt | LVAARDSIESIKDRLLDLNKKQGT |
| | tttctgtgccgcaagctgttcatctccggcaaacacacccaaaccccgaaac | DTVIRDATIESIRDEKLKSRMLNN |
| | gcctggctagactgatcgccaccagcctgagcaatgctaccggcatccgtc | PDRAAYYLIEYVQQSIKYGSNEE |
| | cgcagatcaagtctatcccgcctaagtttgtgtatttttaccgcaacgccgtgc | VYHPTAIEMKQMRDEFVNDDPEL |
| | tgcccgactttaacaaatacgaagacaaaaagaacggtggtgtggtgaacc | YVTEDKELRKQVPNIFGMRIQTG |
| | acattattgactctgcgctgttaggctgcgattttccgtcgccgcactatctgtt | QSESQISFHRVCTQGDTVHHHHY |
| | gtctggtcgttactccggttgtgagaagtaccgtgaattcgttgaccgcttga | MQDIGIRHIAIVVHIDKKGKKQN |
| | aggattcctttgtcgcggtggacgacagcggtatgccgatttgcattccgatt | QAITVTRQGKVYIGLGTARKSVD |
| | agaagcgaaacccaattttttcaagaacaaatcgacaagcgtttgccgggtt | RSFGMTLQGRAFGEKGSRKKWD |
| | actctaaaattgacatgagcaagatgaactggaatagcaaacgcaagatcg | RQWHREFRAFCKATWNVERIHIL |
| | ttagccactaccaaccgcagcgtaaagttggttataaactggtcgcggcgc | TAGCVFIDTDGTTYFVRNMDSQD |
| | gtgactctatcgagtctatcaaggaccgtttgctggacctaaacaagaaaca | KGIRIATAKRKFVEIERVFKSPHA |
| | aggcacggatacggttattcgtgacgcgaccatcgagagcattcgtgatga | YQKYLSKMNAITK (SEQ ID |
| | aaagctcaagtctcggatgctgaacaacccggaccgggctgcttactacttg | NO: 86) |
| | attgagtatgtgcagcaaagcatcaagtacgggtccaacgaagaggtttac | |
| | catccgaccgcaattgagatgaaacagatgcgtgacgagttcgtaaatgat | |
| | gatccagaactgtacgtcaccgaggacaaggagttgagaaagcaggttcc | |
| | gaacatttttggcatgcgtatccagactggccagagcgaaagccagatctct | |
| | ttccaccgtgtgtgcacccagggtgataccgttcatcaccaccattacatgca | |
| | ggacatcggcattcgtcatattgcgattgttgttcacattgacaaaaaaggtaa | |
| | aaagcagaaccaggcaatcactgtgacccgtcaaggtaaggtctacatcg | |
| | gcctgggtacggcgcgtaagagcgttgatcgctcctttggtatgaccctcca | |
| | gggccgtgctttcggtgaaaagggttcccgcaaaaagtgggatcgtcaatg | |
| | gcatcgggagttccgtgcgttctgcaaggctacctggaatgtcgagcgtatt | |
| | catatcctgaccgcaggctgcgtgttcatcgacactgatggcaccacctactt | |
| | cgttcgcaatatggatagccaggacaaaggcatccgcattgcgactgcgaa | |
| | acgtaaattcgtcgaaatcgagcgtgtttttaaaagcccgcacgcctatcaga | |
| | aatacctcagcaagatgaatgccatcaccaag (SEQ ID NO: 85) | |
| D25A (putative | atggatagggagtatctattagaaaaaatacgcgactgtaatccggatgaaa | MDREYLLEKIRDCNPDEIFTSFQI |
| RuvC-knockout) | tcttcactagcttccaaattGCGaccggcgcgaagtactatggttgcaccg | ATGAKYYGCTVLVKLPGVQKPI |
| | tgttggttaaactaccgggggtacagaaaccgattatggtcatcgcggatgt | MVIADVLTAIPKIEDIVAARSLNR |
| | gctgacggctattccaaagatcgaagacattgttgccgctcgttcgctgaatc | GTRRTNKSKKRRNRKLRAAIGHI |
| | gcggtacacgtcgcaccaacaagtcgaagaagcgtcgcaaccgcaagct | EEETSAEDKRTRKSREEVLDKLK |
| | gcgtgcggcgattggtcatatcgacggcgcagataaagttttggcgtattgt | VVVRNEIPDEQQDFVYKTCEKLL |
| | aaacgtcgtggcgtgtcctatgctttcgaccaagacgaagaagaaacctca | NAEERKCRIKNRNPSKCDWEGCN |
| | gctgaagataagcgcacccgtaagtcgcgtgaggaggttctggacaaattg | QNVPASRNAIFETLEQTLYPWLT |
| | aaggtggttgttcgaaatgagatcccggatgagcagcaggacttcgtgtac | QIFRSSKSYVRLENELNECVKYLN |
| | aagacgtgcgagaaactgctgaacgcggaagagcggaaatgccgtatca | SLDYRNKYSIEQTDDEEKQLDKQ |
| | aaaaccgtaacccgtcgaaatgcgactgggaaggttgcaatcaaaacgtg | LNNELNKIRDIVKSHASKEHYREF |
| | ccggcatcgcgaaacgcgatcttcgaaaccctggaacagaccttatatccg | SHYWTKYYRKQLANILKSSGTGR |
| | tggctgacgcagatttttcgcagctctaagtcctacgtacgtctggaaaacga | TTFCRQHSSMYVAKLIAGEVIPV |
| | gcttaacgagtgcgttaaatacttaaacagcctggattatcgtaataaataca | KPEIYLKDIEGAAQKLTYAKLESY |
| | gtatagagcagacggacgatgaggagaagcagctggataaacaattaaac | LLHSVLPTLKTIGITKIDSVCVESA |
| | aatgaactgaacaaaatccgcgacatcgtgaaatctcacgcatccaaagaa | SGSSPSKAKKQKRSDKAKKQKVS |
| | cactaccgcgaattcagtcactactggaccaagtattaccgtaaacaactag | NKTKKQKCSDEEFQQGPMFKFDS |
| | ctaatattttgaaatctagcggcaccggtcgtactacgttttgtcgtcaacattc | RKDMLLAEFDKLCAYCGEPLTGS |
| | ctccatgtatgtagcgaaacttattgccggagaagtgataccagtgaaaccc | YIENEHILPRSRFPYDSYFNILPAC |
| | gaaatctatctgaaagatatcgagggtgcagcgcagaagttgacctatgca | EKCNRRKGARTPYEAGMTISTKA |
| | aagttggagagttacctgctgcatagcgttctgccaaccctgaaaacgattg | LDAFDRYIKKKKFVHPSIWVKRN |
| | gcatcaccaaaattgattccgtgtgtgttgaaagcgcgagcggtagctctcc | LLSGMTHQTNGDAERELRFLCRK |
| | gagcaaagccaaaaaacagaagcgttcagacaaggcgaagaagcaaaa | LFISGKHTQTPKRLARLIATSLSN |
| | agtgagcaataaaaccaaaaaacagaagtgcagcgatgaggagttccaac | ATGIRPQIKSIPPKFVYFYRNAVLP |
| | aaggcccgatgtttaagttcgacagccgcaaagacatgctgctggcggaat | DFNKYEDKKNGGVVNHIIDSALL |
| | tcgataagctgtgtgcatattgcggtgaaccgctcaccggtagctacattga | GCDFPSPHYLLSGRYSGCEKYRE |
| | gaacgagcacatcctgccgcgtagtcggtttccgtatgatagctactttaatat | FVDRLKDSFVAVDDSGMPICIPIR |
| | tctgccggcgtgtgagaagtgcaaccggcgaaagggcgcccgtacccctt | SETQFFQEQIDKRLPGYSKIDMSK |
| | atgaagcgggtatgaccattagcacgaaagcgctggatgcattcgatcgtta | MNWNSKRKIVSHYQPQRKVGYK |
| | tatcaagaagaaaaagttcgtgcacccgtccatctgggttaaacgtaacctg | LVAARDSIESIKDRLLDLNKKQGT |
| | ctgagcggtatgacccatcagacgaacggcgacgccgaacgcgagctgc | DTVIRDATIESIRDEKLKSRMLNN |
| | gttttctgtgccgcaagctgttcatctccggcaaacacacccaaaccccgaa | PDRAAYYLIEYVQQSIKYGSNEE |
| | acgcctggctagactgatcgccaccagcctgagcaatgctaccggcatcc | VYHPTAIEMKQMRDEFVNDDPEL |
| | gtccgcagatcaagtctatcccgcctaagtttgtgtatttttaccgcaacgccg | YVTEDKELRKQVPNIFGMRIQTG |
| | tgctgcccgactttaacaaatacgaagacaaaaagaacggtggtgtggtga | QSESQISFHRVCTQGDTVHHHHY |
| | accacattattgactctgcgctgttaggctgcgattttccgtcgccgcactatc | MQDIGIRHIAIVVHIDKKGKKQN |
| | tgttgtctggtcgttactccggttgtgagaagtaccgtgaattcgttgaccgctt | QAITVTRQGKVYIGLGTARKSVD |
| | gaaggattcctttgtcgcggtggacgacagcggtatgccgatttgcattccg | RSFGMTLQGRAFGEKGSRKKWD |
| | attagaagcgaaacccaattttttcaagaacaaatcgacaagcgtttgccgg | RQWHREFTAFCKATWNVERIHIL |
| | gttactctaaaattgacatgagcaagatgaactggaatagcaaacgcaagat | TAGCVFIDTDGTTYFVRNMDSQD |
| | cgttagccactaccaaccgcagcgtaaagttggttataaactggtcgcggc | KGIRIATAKRKFVEIERVFKSPHA |
| | gcgtgactctatcgagtctatcaaggaccgtttgctggacctaaacaagaaa | YQKYLSKMNAITK (SEQ ID |
| | caaggcacggatacggttattcgtgacgcgaccatcgagagcattcgtgat | NO: 88) |
| | gaaaagctcaagtctcggatgctgaacaacccggaccgggctgcttactac | |
| | ttgattgagtatgtgcagcaaagcatcaagtacgggtccaacgaagaggttt | |
| | accatccgaccgcaattgagatgaaacagatgcgtgacgagttcgtaaatg | |
| | atgatccagaactgtacgtcaccgaggacaaggagttgagaaagcaggttc | |
| | cgaacatttttggcatgcgtatccagactggccagagcgaaagccagatctc | |
| | tttccaccgtgtgtgcacccagggtgataccgttcatcaccaccattacatgc | |
| | aggacatcggcattcgtcatattgcgattgttgttcacattgacaaaaaaggta | |
| | aaaagcagaaccaggcaatcactgtgacccgtcaaggtaaggtctacatcg | |
| | gcctgggtacggcgcgtaagagcgttgatcgctcctttggtatgaccctcca | |
| | gggccgtgctttcggtgaaaagggttcccgcaaaaagtgggatcgtcaatg | |
| | gcatcgggagttccgtgcgttctgcaaggctacctggaatgtcgagcgtatt | |
| | catatcctgaccgcaggctgcgtgttcatcgacactgatggcaccacctactt | |
| | cgttcgcaatatggatagccaggacaaaggcatccgcattgcgactgcgaa | |
| | acgtaaattcgtcgaaatcgagcgtgtttttaaaagcccgcacgcctatcaga | |
| | aatacctcagcaagatgaatgccatcaccaag (SEQ ID NO: 87) | |
| E371A (putative | atggatagggagtatctattagaaaaaatacgcgactgtaatccggatgaaa | MDREYLLEKIRDCNPDEIFTSFQI |
| RuvC-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaagcgtcgcaaccgcaagctgc | DGADKVLAYCKRRGVSYAFDQD |
| | gtgcggcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQDFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVASA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | RKDMLLAEFDKLCAYCGEPLTGS |
| | atctatctgaaagatatcgagggtgcagcgcagaagttgacctatgcaaagt | YIENEHILPRSRFPYDSYFNILPAC |
| | tggagagttacctgctgcatagcgttctgccaaccctgaaaacgattggcat | EKCNRRKGARTPYEAGMTISTKA |
| | caccaaaattgattccgtgtgtgttGCGagcgcgagcggtagctctccga | LDAFDRYIKKKKFVHPSIWVKRN |
| | gcaaagccaaaaaacagaagcgttcagacaaggcgaagaagcaaaaagt | LLSGMTHQTNGDAERELRFLCRK |
| | gagcaataaaaccaaaaaacagaagtgcagcgatgaggagttccaacaa | LFISGKHTQTPKRLARLIATSLSN |
| | ggcccgatgtttaagttcgacagccgcaaagacatgctgctggcggaattc | ATGIRPQIKSIPPKFVYFYRNAVLP |
| | gataagctgtgtgcatattgcggtgaaccgctcaccggtagctacattgaga | DFNKYEDKKNGGVVNHIIDSALL |
| | acgagcacatcctgccgcgtagtcggtttccgtatgatagctactttaatattc | GCDFPSPHYLLSGRYSGCEKYRE |
| | tgccggcgtgtgagaagtgcaaccggcgaaagggcgcccgtaccccttat | FVFRLKDSFVAVDDSGMPICIPIR |
| | gaagcgggtatgaccattagcacgaaagcgctggatgcattcgatcgttata | SETQFFQEQIDKRLPGYSKIDMSK |
| | tcaagaagaaaaagttcgtgcacccgtccatctgggttaaacgtaacctgct | MNWNSKRKIVSHYQPQRKVGYK |
| | gagcggtatgacccatcagacgaacggcgacgccgaacgcgagctgcgt | LVAARDSIESIKDRLLDLNKKQGT |
| | tttctgtgccgcaagctgttcatctccggcaaacacacccaaaccccgaaac | DTVIRDATIESIRDEKLKSRMLNN |
| | gcctggctagactgatcgccaccagcctgagcaatgctaccggcatccgtc | PDRAAYYLIEYVQQSIKYGSNEE |
| | cgcagatcaagtctatcccgcctaagtttgtgtatttttaccgcaacgccgtgc | VYHPTAIEMKQMRDEFVNDDPEL |
| | tgcccgactttaacaaatacgaagacaaaaagaacggtggtgtggtgaacc | YVTEDKELRKQVPNIFGMRIQTG |
| | acattattgactctgcgctgttaggctgcgattttccgtcgccgcactatctgtt | QSESQISFHRVCTQGDTVHHHHY |
| | gtctggtcgttactccggttgtgagaagtaccgtgaattcgttgaccgcttga | MQDIGIRHIAIVVHIDKKGKKQN |
| | aggattcctttgtcgcggtggacgacagcggtatgccgatttgcattccgatt | QAITVTRQGKVYIGLGTARKSVD |
| | agaagcgaaacccaattttttcaagaacaaatcgacaagcgtttgccgggtt | RSFGMTLQGRAFGEKGSRKKWD |
| | actctaaaattgacatgagcaagatgaactggaatagcaaacgcaagatcg | RQWHREFRAFCKATWNVERIHIL |
| | ttagccactaccaaccgcagcgtaaagttggttataaactggtcgcggcgc | TAGCVFIDTDGTTYFVRNMDSQD |
| | gtgactctatcgagtctatcaaggaccgtttgctggacctaaacaagaaaca | KGIRIATAKRKFVEIERVFKSPHA |
| | aggcacggatacggttattcgtgacgcgaccatcgagagcattcgtgatga | YQKYLSKMNAITK (SEQ ID |
| | aaagctcaagtctcggatgctgaacaacccggaccgggctgcttactacttg | NO: 90) |
| | attgagtatgtgcagcaaagcatcaagtacgggtccaacgaagaggtttac | |
| | catccgaccgcaattgagatgaaacagatgcgtgacgagttcgtaaatgat | |
| | gatccagaactgtacgtcaccgaggacaaggagttgagaaagcaggttcc | |
| | gaacatttttggcatgcgtatccagactggccagagcgaaagccagatctct | |
| | ttccaccgtgtgtgcacccagggtgataccgttcatcaccaccattacatgca | |
| | ggacatcggcattcgtcatattgcgattgttgttcacattgacaaaaaaggtaa | |
| | aaagcagaaccaggcaatcactgtgacccgtcaaggtaaggtctacatcg | |
| | gcctgggtacggcgcgtaagagcgttgatcgctcctttggtatgaccctcca | |
| | gggccgtgctttcggtgaaaagggttcccgcaaaaagtgggatcgtcaatg | |
| | gcatcgggagttccgtgcgttctgcaaggctacctggaatgtcgagcgtatt | |
| | catatcctgaccgcaggctgcgtgttcatcgacactgatggcaccacctactt | |
| | cgttcgcaatatggatagccaggacaaaggcatccgcattgcgactgcgaa | |
| | acgtaaattcgtcgaaatcgagcgtgtttttaaaagcccgcacgcctatcaga | |
| | aatacctcagcaagatgaatgccatcaccaag (SEQ ID NO: 89) | |
| D604A (putative | atggatagggagtatctattagaaaaaatacgcgactgtaatccggatgaaa | MDREYLLEKIRDCNPDEIFTSFQI |
| RuvC-knockout) | tcttcactagcttccaaattgacaccggcgcgaagtactatggttgcaccgtg | DTGAKYYGCTVLVKLPGVQKPI |
| | ttggttaaactaccgggggtacagaaaccgattatggtcatcgcggatgtgc | MVIADVLTAIPKIEDIVAARSLNR |
| | tgacggctattccaaagatcgaagacattgttgccgctcgttcgctgaatcgc | GTRRTNKSKKRRNRKLRAAIGHI |
| | ggtacacgtcgcaccaacaagtcgaagaacgtcgcaaccgcaagctgc | DGADKVLAYCKRRGVSYAFDQD |
| | gtgcggcgattggtcatatcgacggcgcagataaagttttggcgtattgtaaa | EEETSAEDKRTRKSREEVLDKLK |
| | cgtcgtggcgtgtcctatgctttcgaccaagacgaagaagaaacctcagct | VVVRNEIPDEQQDFVYKTCEKLL |
| | gaagataagcgcacccgtaagtcgcgtgaggaggttctggacaaattgaa | NAEERKCRIKNRNPSKCDWEGCN |
| | ggtggttgttcgaaatgagatcccggatgagcagcaggacttcgtgtacaa | QNVPASRNAIFETLEQTLYPWLT |
| | gacgtgcgagaaactgctgaacgcggaagagcggaaatgccgtatcaaa | QIFRSSKSYVRLENELNECVKYLN |
| | aaccgtaacccgtcgaaatgcgactgggaaggttgcaatcaaaacgtgcc | SLDYRNKYSIEQTDDEEKQLDKQ |
| | ggcatcgcgaaacgcgatcttcgaaaccctggaacagaccttatatccgtg | LNNELNKIRDIVKSHASKEHYREF |
| | gctgacgcagatttttcgcagctctaagtcctacgtacgtctggaaaacgag | SHYWTKYYRKQLANILKSSGTGR |
| | cttaacgagtgcgttaaatacttaaacagcctggattatcgtaataaatacagt | TTFCRQHSSMYVAKLIAGEVIPV |
| | atagagcagacggacgatgaggagaagcagctggataaacaattaaacaa | KPEIYLKDIEGAAQKLTYAKLESY |
| | tgaactgaacaaaatccgcgacatcgtgaaatctcacgcatccaaagaaca | LLHSVLPTLKTIGITKIDSVCVESA |
| | ctaccgcgaattcagtcactactggaccaagtattaccgtaaacaactagcta | SGSSPSKAKKQKRSDKAKKQKVS |
| | atattttgaaatctagcggcaccggtcgtactacgttttgtcgtcaacattcctc | NKTKKQKCSDEEFQQGPMFKFDS |
| | catgtatgtagcgaaacttattgccggagaagtgataccagtgaaacccgaa | RKDMLLAEFDKLCAYCGEPLTGS |
| | atctatctgaaagatatcgagggtgcagcgcagaagttgacctatgcaaagt | YIENEHILPRSRFPYDSYFNILPAC |
| | tggagagttacctgctgcatagcgttctgccaaccctgaaaacgattggcat | EKCNRRKGARTPYEAGMTISTKA |
| | caccaaaattgattccgtgtgtgttgaaagcgcgagcggtagctctccgagc | LDAFDRYIKKKKFVHPSIWVKRN |
| | aaagccaaaaaacagaagcgttcagacaaggcgaagaagcaaaaagtga | LLSGMTHQTNGDAERELRFLCRK |
| | gcaataaaaccaaaaaacagaagtgcagcgatgaggagttccaacaagg | LFISGKHTQTPKRLARLIATSLSN |
| | cccgatgtttaagttcgacagccgcaaagacatgctgctggcggaattcgat | ATGIRPQIKSIPPKFVYFYRNAVLP |
| | aagctgtgtgcatattgcggtgaaccgctcaccggtagctacattgagaacg | DFNKYEDKKNGGVVNHIIASALL |
| | agcacatcctgccgcgtagtcggtttccgtatgatagctactttaatattctgc | GCDFPSPHYLLSGRYSGCEKYRE |
| | cggcgtgtgagaagtgcaaccggcgaaagggcgcccgtaccccttatgaa | FVDRLKDSFVAVDDSGMPICIPIR |
| | gcgggtatgaccattagcacgaaagcgctggatgcattcgatcgttatatca | SETQFFQEQIDKRLPGYSKIDMSK |
| | agaagaaaaagttcgtgcacccgtccatctgggttaaacgtaacctgctgag | MNWNSKRKIVSHYQPQRKVGYK |
| | cggtatgacccatcagacgaacggcgacgccgaacgcgagctgcgttttct | LVAARDSIESIKDRLLDLNKKQGT |
| | gtgccgcaagctgttcatctccggcaaacacacccaaaccccgaaacgcct | DTVIRDATIESIRDEKLKSRMLNN |
| | ggctagactgatcgccaccagcctgagcaatgctaccggcatccgtccgc | PDRAAYYLIEYVQQSIKYGSNEE |
| | agatcaagtctatcccgcctaagtttgtgtatttttaccgcaacgccgtgctgc | VYHPTAIEMKQMRDEFVNDDPEL |
| | ccgactttaacaaatacgaagacaaaaagaacggtggtgtggtgaaccaca | YVTEDKELRKQVPNIFGMRIQTG |
| | ttattGCGtctgcgctgttaggctgcgattttccgtcgccgcactatctgttg | QSESQISFHRVCTQGDTVHHHHY |
| | tctggtcgttactccggttgtgagaagtaccgtgaattcgttgaccgcttgaa | MQDIGIRHIAIVVHIDKKGKKQN |
| | ggattcctttgtcgcggtggacgacagcggtatgccgatttgcattccgatta | QAITVTRQGKVYIGLGTARKSVD |
| | gaagcgaaacccaattttttcaagaacaaatcgacaagcgtttgccgggtta | RSFGMTLQGRAFGEKGSRKKWD |
| | ctctaaaattgacatgagcaagatgaactggaatagcaaacgcaagatcgtt | RQWHREFRAFCKATWNVERIHIL |
| | agccactaccaaccgcagcgtaaagttggttataaactggtcgcggcgcgt | TAGCVFIDTDGTTYFVRNMDSQD |
| | gactctatcgagtctatcaaggaccgtttgctggacctaaacaagaaacaag | KGIRIATAKRKFVEIERVFKSPHA |
| | gcacggatacggttattcgtgacgcgaccatcgagagcattcgtgatgaaa | YQKYLSKMNAITK (SEQ ID |
| | agctcaagtctcggatgctgaacaacccggaccgggctgcttactacttgat | NO: 92) |
| | tgagtatgtgcagcaaagcatcaatacgggtccaacgaagaggtttacca | |
| | tccgaccgcaattgagatgaaacagatgcgtgacgagttcgtaaatgatgat | |
| | ccagaactgtacgtcaccgaggacaaggagttgagaaagcaggttccgaa | |
| | catttttggcatgcgtatccagactggccagagcgaaagccagatctctttcc | |
| | accgtgtgtgcacccagggtgataccgttcatcaccaccattacatgcagga | |
| | catcggcattcgtcatattgcgattgttgttcacattgacaaaaaaggtaaaaa | |
| | gcagaaccaggcaatcactgtgacccgtcaaggtaaggtctacatcggcct | |
| | gggtacggcgcgtaagagcgttgatcgctcctttggtatgaccctccaggg | |
| | ccgtgctttcggtgaaaagggttcccgcaaaaagtgggatcgtcaatggcat | |
| | cgggagttccgtgcgttctgcaaggctacctggaatgtcgagcgtattcatat | |
| | cctgaccgcaggctgcgtgttcatcgacactgatggcaccacctacttcgtt | |
| | cgcaatatggatagccaggacaaaggcatccgcattgcgactgcgaaacg | |
| | taaattcgtcgaaatcgagcgtgtttttaaaagcccgcacgcctatcagaaat | |
| | acctcagcaagatgaatgccatcaccaag (SEQ ID NO: 91) | |

A linear DNA template sequence encoding an RNA guide was designed and ordered (IDT) with a T7 promoter upstream and a T7Te terminator sequence downstream of the guide. The RNA guide was designed to be specific to a previously tested target sequence, described in Example 5, within the coding exon of EMX1 with a 5′-ARCC-3′ PAM sequence (the PAM is 3′ of the target sequence). The sequence of the encoded RNA guide and its individual components are shown in Table 20 below.

TABLE 20
RNA Sequences

| Components | RNA sequence | SEQ ID |
|---|---|---|
| Reference | guuguauucggaugggaguaaaagguugaguacaccuggaaacaaaacgacaua | 63 |
| sgRNA scaffold | aagguguucucccaucuguguacaaagagcagauauaugaguugauaaguaaua | |
| | uguugcacguauggacuuugacagucuguaccucugcgugaaagacauauugc | |
| | guuauucauauaucugugagucuguucuaaauuuaaagguuuagggaa | |
| EMX1-T4 | AUGACUCCAGGCCUCCCCAA | 93 |
| spacer | | |
| T7Te terminator | ggcucaccuucgggugggccuuucugcg | 39 |
| T7 transcribed | gAUGACUCCAGGCCUCCCCAAguuguauucggaugggaguaaaa | 94 |
| EMX1-T4 | gguugaguacaccuggaaacaaaacgacauaaagguguucucccaucuguguac | |
| sgRNA | aaagagcagauauaugaguugauaaguaauauguugcacguauggacuuugaca | |
| | gucuguaccucugcgugaaagacauauugcguuauucauauaucugugagucu | |
| | guucuaaauuuaaagguuuagggaaggcucaccuucgggugggccuuucugcg | |

A DNA target was designed and ordered as a synthesized linear DNA fragment. The target sequence from EMX1 and 10 bases upstream and downstream within the exon was flanked by 100 bases of unrelated sequence upstream and 200 bases of unrelated sequence downstream. The extra sequence was added so that the cleaved and uncleaved products would separate well on a gel. The target and non-target strands were labelled with 5′ IR800 and 5′ IR700 labels, respectively, through PCR amplification using labelled primers. The sequences of the DNA target, the individual components of the DNA target, and the labelled PCR primers are in Table 21.

TABLE 21
Target gBlock and Primer Sequences

| Components | DNA sequence | SEQ ID |
|---|---|---|
| EMX1-T4 target | ATGACTCCAGGCCTCCCCAA | 55 |
| EMX1-T4 | CTGTGGGGCC | 95 |
| upstream 10 nt | | |
| EMX1-T4 | AGCCTGGCCA | 96 |
| downstream 10 nt | | |
| 100 nt upstream | gctcaaagctttagactggaggtaccgtagtactatgagcttaatcgcgctgagggtgattt | 97 |
| sequence | gggtgttcgtctcatatcttcgattatcggacagtagt | |
| 200 nt | agtagtatcggaccggttggcgaaccacagcgtataacgagacatggactcgcattccct | 98 |
| downstream | accttaagcgggcggttttggagctcggaattcatgtaaactcgcccgtttaacataatcta | |
| sequence | gatggatcttgcgcgcaccgcccgaacaccactcgccacaaaaaaccgccggaacgtc | |
| | caaaaglacgggttttgctg | |
| Full target | gctcaaagctttagactggaggtaccgtagtactatgagcttaatcgcgctgagggtgattt | 99 |
| gblock | gggtgttcgtctcatatcttcgattatcggacagtagtCTGTGGGGCCATGAC | |
| | TCCAGGCCTCCCCAAAGCCTGGCCAagtagtatcggaccggttgg | |
| | cgaaccacagcgtataacgagacatggactcgcattccctaccttaagcgggcggttttg | |
| | gagctcggaattcatgtaaactcgcccgtttaacataatctagatggatottgcgcgcaccg | |
| | cccgaacaccactcgccacaaaaaaccgccggaacgtccaaaagtacgggttttgctg | |
| Forward primer | /5IRD700/GCTCAAAGCTTTAGACTGGAGGTACCG | 100 |
| Reverse primer | /5IRD800/CAGCAAAACCCGTACTTTTGGACG | 101 |

Cleavage activity of the reference Nuclease S (SEQ ID NO: 49) and each of the putative nickase variants thereof was assessed using in vitro cleavage assays. Each polypeptide was individually co-expressed with the RNA guide in vitro by incubating the plasmid encoding the protein of interest from Table 19 and linear DNA template for the T7 transcribed EMX1-T4 sgRNA from Table 20 in a PURExpress® solution (NEB) containing SUPERase⋅In™ RNase Inhibitor (Invitrogen) for 2 hours at 37° C. The unpurified polypeptide/RNA solution was then diluted into a solution of 1×NEB Buffer 2 (NEB) containing approximately 1 ng/μl of the labelled DNA target amplicon. The solution was then incubated for 1 hour at 37° C. Reactions were stopped by incubating with RNase Cocktail™ (Invitrogen; approximately 1 U/μl final concentration) at 37° C. for 15 minutes, followed by incubating with Proteinase K (NEB; approximately 0.04 U/μl final concentration) at 55° C. for 30 minutes. The DNA was then purified using CleanNGS DNA & RNA Clean-Up Magnetic Beads (Bulldog Bio).

The cleaved and uncleaved products of the target and non-target strands were separated by running the samples on a 10% TBE-Urea PAGE gel. The gel was imaged using a LI-COR Odysssey M imaging system using the 800 nm and 700 nm channels to visualize the 5′ IR800 and 5′ IR700 labels on the target and non-target strands of the target DNA substrate. Band intensities were quantified using ImageJ software.

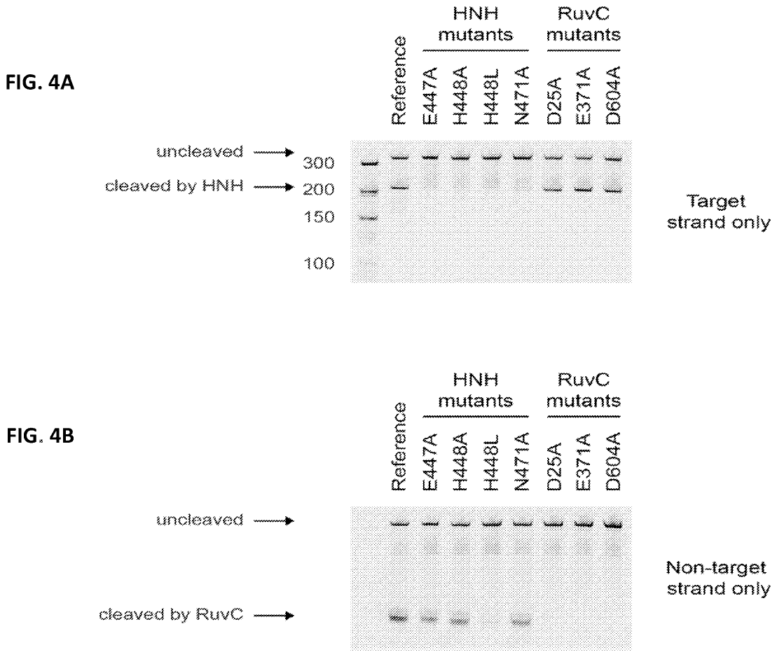

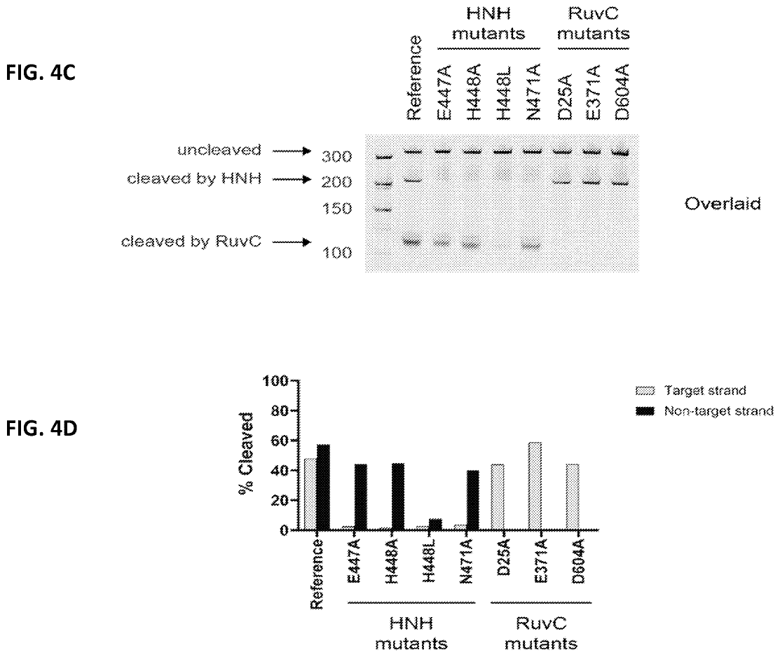

Gel images are shown in FIGS. 4A-4C, and quantification of the percent of cleaved target and non-target strands are shown in FIG. 4D. The uncleaved, HNH-cleaved, and RuvC-cleaved strands are indicated. FIG. 4A is a gel image captured using the 800 nm channel showing cleavage of the target strand. FIG. 4B is a gel image captured using the 700 nm channel showing cleavage of the non-target strand. FIG. 4C is an overlay of the gel images from FIG. 4A and FIG. 4B. As shown in FIG. 4A-4D, the reference Nuclease S (SEQ ID NO: 49) cleaved both the target strand and the non-target strand, as expected. Three of the four HNH-knockout nickase constructs (E447A, H448A, and N471A) showed significantly decreased activity on the target strand while retaining activity on the non-target strand. Each of the three RuvC-knockout nickase constructs (D25A, E371A, and D604A) showed significantly decreased activity on the non-target strand while retaining activity on the target strand (FIG. 4A-4D).

This Example thus shows that HNH-knockout nickases and RuvC-knockout nickases were successfully engineered. The H448A variant of Nuclease S (SEQ ID NO: 49) is used to install edits into human gene targets.

### Example 9: CRISPR Nuclease Mediated Editing of Human Target Sequences in HEK293T Cells

This Example describes the genomic editing of the AAVS1, EMX1, and VEGFA genes by the CRISPR nuclease of SEQ ID NO: 64 (Nuclease H) introduced into cells by lipid-based transient transfection into the HEK293T cell line.

Nuclease H was tagged with N-terminal SV40 nuclear localization sequence and a C-terminal nucleoplasmin nuclear localization sequence and converted to a human codon-optimized DNA sequence, synthesized, and cloned into a pcDNA3.1 vector (Invitrogen), containing a CMV promoter for expression. The reference and NLS-tagged sequences used are in Table 14. Plasmids were purified using a midiprep kit.

RNA guides were designed and cloned and into a pUC19 plasmid following the U6 PolIII promoter and terminated with a 6×polyT sequence. RNA guides were designed to be specific to target sequences within the coding exons of AAVS1, EMX1, and VEGFA with 5′-NRC-3′ PAM sequences (the PAM sequence is on the 3′ end of the target sequence). See all RNA guide sequences in Table 15. The U6 PolIII promoter uses a +1 G at the start of the transcript (i.e., the 5′ end of the RNA) for more efficient transcription that is excluded from the sequences described in Table 15. Plasmids were purified using a midiprep kit.

TABLE 14
Amino Acid Sequences

| Component | Amino acid sequence | SEQ ID NO |
|---|---|---|
| Reference | MITLGIDYGAAYVGIALVRNHENGHDEPLFAATMVLDARRLK | 64 |
| Nuclease H | EKVENRAALRRLRRTRKTKKRRLRELKDKLLRLGLAPETVSQI | |
| | VNFCERRGYKSLFGQEGDPDDKSRDELTYRFTREEFFQTLEGH | |
| | LSRLLLSPEAKTAALAICERVLNRAGDRRLEVRPLRIENRGASR | |
| | CAWEGCDRVTPRRSNAIEDAITQQLVTYFQSTLKQHQDLLDTL | |
| | KVATAEMGRISHQLRSAVEKGLAPEVKALRKRARDVLKRLRV | |
| | EFTEPEAAEENDEKAWKYVKEGIMNILEKARGRNAYCREHSA | |
| | EYVQTVLAGRAVPFKKTIAESDILSRREQIAFSKLWRYIEARVL | |
| | PLAPEGIDRIVVERTAFDVLAGNRRKIRTTSEKRVEEMYQQGPR | |
| | FGFKDDGAMLREEFGGLCAYCGKPSPDLIEREHVLPRRDFFFDS | |
| | YLNILPACPDCNRQKGRSRPGSAALHISEEAYANYDRYLKRIAG | |
| | QRPVHFLHYEKKGILNLMRDPKRAWDAERYLGLLANNFAAIA | |
| | QSQRGPRPFARLLFSKLAARQNKPPTISFFSGRHTAMYRTIAYP | |
| | GFEKHKDKAEGGTMNHALDAVLLACELPNASVLEARGINVQD | |
| | LETWRRRVRSRAPVPGKDGVPDIPLYEWCVPGFEEVGLNGYV | |
| | EVQMAAMNWNQKDSSTHKQDPYGWSEESNKPTKRSSAVELY | |
| | EKLMNEKADEKVKNIVQTIHHPSLRSAMSQGLKARPPGPAVAE | |
| | AMKEWLRKSVRNSLGKSSCTSHPGDLRRRTDLQNFAENHDAK | |
| | IPRVIGVKRLDTGVQGKIDLERLDPMNGRTLHRYMSDPVNREV | |
| | LLAYRVGSNGQPDFLKPFTVKVKQNLALKTDLRTFQPIPPDLA | |
| | CGVTWGSAASLGDGWSSKLETYLSERGFCSYARIKPCCVVCY | |
| | QDGSQLFIRNFDDNKDFKPSKILKNVVGVKRTPFALRMVPLKV | |
| | LTPQRPK | |
| SV40 NLS | KRTADGSEFESPKKKRKV | 3 |
| Nucleoplasmin | KRPAATKKAGQAKKKK | 4 |
| NLS | | |
| XTEN linker | SGGSSGGSSGSETPGTSESATPESSGGSSGGSS | 5 |
| NLS-tagged | MKRTADGSEFESPKKKRKVITLGIDYGAAYVGIALVRNHENGHD | 65 |
| Reference | EPLFAATMVLDARRLKEKVENRAALRRLRRTRKTKKRRLREL | |
| Nuclease H | KDKLLRLGLAPETVSQIVNFCERRGYKSLFGQEGDPDDKSRDE | |
| | LTYRFTREEFFQTLEGHLSRLLLSPEAKTAALAICERVLNRAGD | |
| | RRLEVRPLRIENRGASRCAWEGCDRVTPRRSNAIEDAITQQLVT | |
| | YFQSTLKQHQDLLDTLKVATAEMGRISHQLRSAVEKGLAPEVK | |
| | ALRKRARDVLKRLRVEFTEPEAAEENDEKAWKYVKEGIMNIL | |
| | EKARGRNAYCREHSAEYVQTVLAGRAVPFKKTIAESDILSRRE | |
| | QIAFSKLWRYIEARVLPLAPEGIDRIVVERTAFDVLAGNRRKIRT | |
| | TSEKRVEEMYQQGPRFGFKDDGAMLREEFGGLCAYCGKPSPD | |
| | LIEREHVLPRRDFFFDSYLNILPACPDCNRQKGRSRPGSAALHIS | |
| | EEAYANYDRYLKRIAGQRPVHFLHYEKKGILNLMRDPKRAWD | |
| | AERYLGLLANNFAAIAQSQRGPRPFARLLFSKLAARQNKPPTIS | |
| | FFSGRHTAMYRTIAYPGFEKHKDKAEGGTMNHALDAVLLACE | |
| | LPNASVLEARGINVQDLETWRRRVRSRAPVPGKDGVPDIPLYE | |
| | WCVPGFEEVGLNGYVEVQMAAMNWNQKDSSTHKQDPYGWS | |
| | EESNKPTKRSSAVELYEKLMNEKADEKVKNIVQTIHHPSLRSA | |
| | MSQGLKARPPGPAVAEAMKEWLRKSVRNSLGKSSCTSHPGDL | |
| | RRRTDLQNFAENHDAKIPRVIGVKRLDTGVQGKIDLERLDPMN | |
| | GRTLHRYMSDPVNREVLLAYRVGSNGQPDFLKPFTVKVKQNL | |
| | ALKTDLRTFQPIPPDLACGVTWGSAASLGDGWSSKLETYLSER | |
| | GFCSYARIKPCCVVCYQDGSQLFIRNFDDNKDFKPSKILKNVVG | |
| | VKRTPFALRMVPLKVLTPQRPKSGGSSGGSSGSETPGTSESATP | |
| | ESSGGSSGGSSKRPAATKKAGQAKKKK | |

TABLE 15
Target and RNA Guide Sequences

| | | Target/Spacer | |
|---|---|---|---|
| Guide | PAM | Sequence | RNA Guide Sequence* |
| AAVS1- | GAC | ACGACCTGGT | ACGACCUGGUGAACACCUAGguuucaaucaaccugaucucu |
| T3 | | GAACACCTAG | cugcuugcgcugcaggcuucauaagaucaguagguugaaaagagcauccg |
| | | (SEQ ID NO: 66) | uccgacaugcacuccgggauggggcagucccggcucuuggguucccgau |
| | | | gcaguuauguguccgcggaacacggugucggacacgucgcaagacaggau |
| | | | gugagcccauuauccacauuaggaucaauguacgcaugagcaguccgcaau |
| | | | gccggggacgcu (SEQ ID NO: 67) |
| AAVSI- | AAC | GGCTATAATC | GGCUAUAAUCCAUGCUGACCguuucaaucaaccugaucucu |
| T5 | | CATGCTGACC | cugcuugcgcugcaggcuucauaagaucaguagguugaaaagagcauccg |
| | | (SEQ ID NO: 53) | uccgacaugcacuccgggauggggcagucccggcucuuggguucccgau |
| | | | gcaguuauguguccgcggaacacggugucggacacgucgcaagacaggau |
| | | | gugagcccauuauccacauuaggaucaauguacgcaugagcaguccgcaau |
| | | | gccggggacgcu (SEQ ID NO: 68) |
| EMX1-T2 | TAC | AGGTCAGATG | AGGUCAGAUGAUAGCAUAGGguuucaaucaaccugaucuc |
| | | ATAGCATAGG | ucugcuugcgcugcaggcuucauaagaucaguagguugaaaagagcaucc |
| | | (SEQ ID NO: 69) | guccgacaugcacuccgggauggggcagucccggcucuuggguucccga |
| | | | ugcaguuauguguccgcggaacacggugucggacacgucgcaagacagga |
| | | | ugugagcccauuauccacauuaggaucaauguacgcaugagcaguccgcaa |
| | | | ugccggggacgcu (SEQ ID NO: 70) |
| EMX1-T5 | CAC | GAGGCCTGGA | GAGGCCUGGAGUCAUGGCCCguuucaaucaaccugaucucu |
| | | GTCATGGCCC | cugcuugcgcugcaggcuucauaagaucaguagguugaaaagagcauccg |
| | | (SEQ ID NO: 71) | uccgacaugcacuccgggauggggcagucccggcucuuggguucccgau |
| | | | gcaguuauguguccgcggaacacggugucggacacgucgcaagacaggau |
| | | | gugagcccauuauccacauuaggaucaauguacgcaugagcaguccgcaau |
| | | | gccggggacgcu (SEQ ID NO: 72) |
| VEGFA- | AAC | TTCCAGGTGT | UUCCAGGUGUGGACAAUGUCguuucaaucaaccugaucuc |
| T4 | | GGACAATGTC | ucugcuugcgcugcaggcuucauaagaucaguagguugaaaagagcaucc |
| | | (SEQ ID NO: 73) | guccgacaugcacuccgggauggggcagucccggcucuugcgguucccga |
| | | | ugcaguuauguguccgcggaacacggugucggacacgucgcaagacagga |
| | | | ugugagcccauuauccacauuaggaucaauguacgcaugagcaguccgcaa |
| | | | ugccggggacgcu (SEQ ID NO: 74) |
| VEGFA- | GAC | GACTCTTCAA | GACUCUUCAAAUCCUCCCUUguuucaaucaaccugaucucu |
| T6 | | ATCCTCCCTT | cugcuugcgcugcaggcuucauaagaucaguagguugaaaagagcauccg |
| | | (SEQ ID NO: 75) | uccgacaugcacuccgggauggggcagucccggcucuuggguucccgau |
| | | | gcaguuauguguccgcggaacacggugucggacacgucgcaagacaggau |
| | | | gugagcccauuauccacauuaggaucaauguacgcaugagcaguccgcaau |
| | | | gccggggacgcu (SEQ ID NO: 76) |

*Spacer in upper case and scaffold (SEQ ID NO: 77) in lower case

HEK293T cells were transfected with plasmids expressing the Nuclease H polypeptides and the gRNAs provided herein following the methods provided in Example 1 above. Gene editing efficiencies were determined by NGS following the methods also provided in Example 1. The NGS results were analyzed also following the methods provided in Example 1 above.

For each target, indel ratios, referring to the percentage of NGS reads comprising indels, were calculated for each sample and its cognate no protein control. Targets comprising a higher percentage of indels when Nuclease H was included in the transfection were indicative of DNA editing outcomes in the cell.

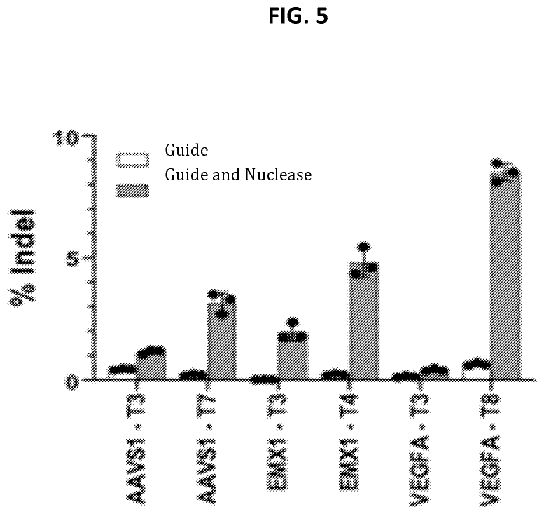

As shown in FIG. 5, each of the six targets tested demonstrated a greater level of indels observed when the Nuclease H (SEQ ID NO: 64) plasmid was present.

### Example 10: Effectiveness of Variant CRISPR Nucleases for Targeting of Mammalian Genes

This Example describes indel assessment on mammalian targets using variants of Nuclease H transfected into HEK293T cells following the methods disclosed in Example 9 above.

Arginine scanning mutagenesis was performed to individually substitute non-arginine residues of the reference Nuclease H of SEQ ID NO: 64 to arginine. SEQ ID NO: 64 is referred to herein as the reference sequence. This resulted in 849 single arginine substitution variants.

Nucleic acids encoding the reference Nuclease H and each Nuclease H variant were then individually cloned into a pcDNA3.1 backbone (Invitrogen™), and the plasmids were maxi-prepped and diluted. The plasmids comprised a CMV promoter, a first NLS (KRTADGSEFESPKKKRKV; SEQ ID NO: 3) upstream of the coding sequence, and an XTEN linker (SGGSSGGSSGSETPGTSESATPESSGGSSGGSS; SEQ ID NO: 5) followed by a second NLS (KRPAATKKAGQAKKKK; SEQ ID NO:4) downstream of the coding sequence. See also Example 9 above.

The RNA guide and target sequences are shown in Table 16. RNA guides were cloned into a pUC19 backbone (New England Biolabs®) following the U6 PolIII promoter and terminated with a 6×polyT sequence. The U6 PolIII uses a +1 G at the start of the transcript (i.e., the 5′ end of the RNA) for more efficient transcription that is excluded from the sequences described in Table 11. The plasmids were then maxi-prepped and diluted.

TABLE 16
Mammalian Targets and Corresponding crRNAs*

| Target | PAM | | RNA Guide |
|---|---|---|---|
| Identifier | Sequence | Target Sequence | Sequence |
| AAVS1 | AAC | SEQ ID NO: 53 | SEQ ID NO: 67 |
| EMX1 | CAC | SEQ ID NO: 70 | SEQ ID NO: 71 |
| VEGFA | GAC | SEQ ID NO: 74 | SEQ ID NO: 75 |

*see Table 15 for sequence details

Indel ratios, referring to the percentage of NGS reads comprising indels, were calculated for the reference and for each variant. The indel ratios used for fold change calculations were the average of two technical replicates. To then calculate fold change in indel ratios, the indel ratio for each variant was divided by the indel ratio for the reference. Table 17 shows fold change in indel ratios for each target. Numbering is relative to the reference nuclease of SEQ ID NO: 64 (i.e., without an NLS).

As shown in Table 17, 9 variants with single arginine substitutions (left column) were characterized as yielding at least a 2.5× increase in indel ratio relative to the reference indel ratio, when averaged across all targets (right column).

TABLE 17
Fold Change in Indel Ratios*

| | | | | Average Fold |
|---|---|---|---|---|
| Substitution | AAVS1 | EMX1 | VEGFA | Change |
| L54R | 16.62 | 2.58 | 1.05 | 6.75 |
| L323R | 5.91 | 3.18 | 2.57 | 3.89 |
| D912R | 4.21 | 3.69 | 2.09 | 3.33 |
| D321R | 4.48 | 1.82 | 2.98 | 3.09 |
| T783R | 2.48 | 4.33 | 1.44 | 2.75 |
| L51R | 4.24 | 2.07 | 1.70 | 2.67 |
| E43R | 3.40 | 2.65 | 1.74 | 2.59 |
| S680R | 3.45 | 2.00 | 2.18 | 2.55 |
| A170R | 3.24 | 1.73 | 2.57 | 2.51 |

* Variant indel ratio/Reference indel ratio

20 variants were analyzed as having indel ratios 2-2.5× of the reference indel ratios, when averaged across all targets: N700R, Q664R, T704R, V311R, A50R, D179R, E638R, E46R, F547R, D845R, N913R, L905R, G869R, A38R, E792R, P893R, A11R, A174R, T661R, and E111R.

329 variants were analyzed as having indel ratios 1-1.9× of the reference indel ratios, when averaged across all targets: E587R, N47R, N674R, N600R, E646R, Q648R, G394R, T124R, S673R, A292R, A857R, G507R, A841R, L846R, L794R, G475R, P262R, Q786R, D37R, S660R, V860R, A318R, T260R, D603R, Q703R, L73R, Q123R, N23R, E571R, A694R, D501R, S135R, V257R, S659R, D110R, K286R, L840R, N590R, N366R, E266R, L112R, L935R, E67R, E672R, A596R, N512R, D625R, K62R, Q476R, K663R, L628R, Q656R, T861R, H128R, A265R, Q602R, S130R, M797R, A515R, F116R, S712R, K693R, A651R, Q84R, N763R, T574R, E258R, L755R, T759R, K675R, G640R, C858R, S186R, K71R, V35R, N87R, E90R, K699R, E380R, S867R, L868R, D782R, S747R, A865R, K843R, D870R, E120R, E630R, A299R, E766R, H21R, A287R, E595R, V305R, D105R, S545R, V617R, S750R, Q384R, S231R, A866R, D665R, F313R, K246R, D103R, A730R, K771R, S374R, S592R, H227R, S108R, S919R, Q716R, T878R, S864R, L134R, K44R, N511R, G744R, S873R, V241R, K688R, E300R, D426R, K95R, T848R, C748R, S320R, T140R, K253R, K369R, Q100R, S887R, D106R, L781R, A144R, G573R, K69R, K929R, H768R, K376R, K737R, D250R, T606R, P854R, E126R, P136R, T557R, Y114R, G236R, S874R, S881R, L133R, E22R, K569R, G562R, A570R, K620R, Y484R, G127R, E564R, E733R, S613R, N798R, Q825R, S203R, A474R, K657R, M575R, P411R, K745R, K567R, S453R, Y12R, E605R, K920R, K875R, K42R, T316R, N767R, T543R, K410R, D414R, P104R, V942R, H551R, V593R, V82R, A141R, S83R, L244R, D621R, L132R, K892R, Q518R, E729R, D769R, T372R, P413R, A307R, K139R, K837R, E101R, G572R, V820R, K314R, T944R, K923R, T749R, E671R, E461R, L829R, A264R, D70R, V302R, A364R, K949R, H566R, D210R, P541R, G799R, K274R, E119R, A535R, P634R, E263R, G76R, T194R, A78R, G488R, K107R, T113R, Q303R, K315R, Q900R, K540R, D393R, A770R, E234R, N741R, A536R, S822R, P79R, K719R, S412R, W669R, T117R, L237R, Y275R, E270R, E696R, N642R, P722R, A232R, N691R, F828R, D467R, G753R, E278R, H457R, K277R, V149R, L508R, G102R, G24R, G99R, K497R, A591R, K830R, I317R, A650R, V938R, P945R, I599R, V601R, P796R, D695R, K271R, K697R, S715R, Q762R, K914R, A330R, E375R, E80R, W862R, K917R, P948R, A720R, N268R, S298R, M690R, F531R, V86R, L211R, N539R, V233R, A726R, D269R, E296R, G365R, E137R, Q228R, E285R, K941R, T373R, S332R, A220R, L143R, A713R, K61R, E348R, Q946R, A310R, T81R, V354R, S903R, I189R, A218R, G821R, K391R, G598R, G871R, G308R, K471R, P725R, H706R, G635R, and N924R.

The remaining variants (491 variants) resulted in decreased indel ratios relative to the reference indel ratios (fold change in indel ratios of less than 1.0).

Based on this experiment, variants of Nuclease H having the following substitutions (in SEQ ID NO: 64) were selected for further engineering: L54R, L323R, D912R, T783R, E43R, S680R, and A170R.

### Example 11: Effectiveness of Combination CRISPR Nuclease Variants for Targeting of Mammalian Genes

This Example describes indel assessment on mammalian targets using Nuclease H variants, comprising two or more substitutions identified as increasing indel activity in Example 10. 56 combination Nuclease H variants were tested.

Each Nuclease H variant and RNA guide (see Table 16) was cloned as described in Example 9. HEK293T cells were further transfected, followed by NGS analysis, as described in Example 10. For each target, indel ratios, referring to the percentage of NGS reads comprising indels, were calculated for the reference Nuclease H (SEQ ID NO: 64) and for each Nuclease H variant. The indel ratios shown in Table 18 were calculated as the average of two bioreplicates, each of which contained two technical replicates.

TABLE 18
Indel Ratios for Mammalian Targets

| | AAVS1 | EMX1 | VEGFA | Average |
|---|---|---|---|---|
| | Average | Average | Average | Indel Ratio |
| | Indel | Indel | Indel | (all |
| Substitutions | Ratio | Ratio | Ratio | targets) |
| T783R, E43R, A170R | 0.1839 | 0.2632 | 0.2415 | 0.2295 |
| L54R, L323R, T783R | 0.3995 | 0.1618 | 0.0969 | 0.2194 |
| L323R, T783R, E43R | 0.1924 | 0.2955 | 0.1507 | 0.2129 |
| L323R, E43R, A170R | 0.2242 | 0.1711 | 0.2380 | 0.2111 |
| L54R, T783R, A170R | 0.3455 | 0.0825 | 0.1859 | 0.2046 |
| L323R, D912R, A170R | 0.1817 | 0.2397 | 0.1644 | 0.1953 |
| D912R, A170R | 0.1370 | 0.2474 | 0.1867 | 0.1904 |
| E43R, A170R | 0.1530 | 0.1635 | 0.2531 | 0.1899 |
| L54R, E43R, A170R | 0.2804 | 0.0359 | 0.2516 | 0.1893 |
| L54R, D912R, A170R | 0.2552 | 0.1513 | 0.1609 | 0.1891 |
| L54R, L323R, D912R | 0.2920 | 0.1941 | 0.0745 | 0.1869 |
| L323R, E43R, S680R | 0.2117 | 0.1845 | 0.1557 | 0.1840 |
| L54R, L323R, E43R | 0.3291 | 0.0595 | 0.1604 | 0.1830 |
| D912R, T783R, A170R | 0.1325 | 0.2607 | 0.1463 | 0.1798 |
| E43R, S680R, A170R | 0.2010 | 0.1257 | 0.2029 | 0.1765 |
| L54R, L323R, S680R | 0.3150 | 0.0550 | 0.1047 | 0.1582 |
| L54R, S680R, A170R | 0.2844 | 0.0253 | 0.1622 | 0.1573 |
| L323R, T783R, A170R | 0.1355 | 0.1979 | 0.1376 | 0.1570 |
| L54R, L323R | 0.3148 | 0.0785 | 0.0726 | 0.1553 |
| L54R, A170R | 0.2958 | 0.0414 | 0.1286 | 0.1553 |
| L323R, D912R, T783R | 0.1347 | 0.2222 | 0.1061 | 0.1543 |
| L54R, T783R, E43R | 0.2861 | 0.0926 | 0.0825 | 0.1537 |
| D912R, S680R, A170R | 0.1359 | 0.2058 | 0.1166 | 0.1528 |
| L323R, E43R | 0.1402 | 0.1749 | 0.1421 | 0.1524 |
| T783R, A170R | 0.0941 | 0.2077 | 0.1530 | 0.1516 |
| L54R, L323R, A170R | 0.2755 | 0.0462 | 0.1239 | 0.1485 |
| L54R, T783R | 0.2600 | 0.1419 | 0.0390 | 0.1470 |
| L323R, T783R, S680R | 0.1286 | 0.2115 | 0.0890 | 0.1430 |
| D912R, E43R, A170R | 0.1039 | 0.1892 | 0.1307 | 0.1413 |
| T783R, S680R, A170R | 0.1360 | 0.1599 | 0.1248 | 0.1402 |
| L323R, D912R | 0.1244 | 0.1864 | 0.1094 | 0.1401 |
| L323R, S680R, A170R | 0.1782 | 0.0773 | 0.1432 | 0.1329 |
| L54R, D912R | 0.1862 | 0.1730 | 0.0274 | 0.1289 |
| T783R, E43R | 0.0600 | 0.2295 | 0.0915 | 0.1270 |
| L54R, E43R | 0.2369 | 0.0628 | 0.0794 | 0.1263 |
| L54R, T783R, S680R | 0.2619 | 0.0842 | 0.0324 | 0.1261 |
| S680R, A170R | 0.1305 | 0.0867 | 0.1596 | 0.1256 |
| L323R, D912R, E43R | 0.1123 | 0.1605 | 0.0972 | 0.1234 |
| L54R, D912R, T783R | 0.1460 | 0.1862 | 0.0268 | 0.1197 |
| L323R, T783R | 0.0871 | 0.1840 | 0.0815 | 0.1175 |
| L323R, D912R, S680R | 0.0997 | 0.1688 | 0.0839 | 0.1174 |
| T783R, E43R, S680R | 0.0713 | 0.2055 | 0.0742 | 0.1170 |
| L54R, S680R | 0.2369 | 0.0521 | 0.0395 | 0.1095 |
| E43R, S680R | 0.0707 | 0.1364 | 0.0913 | 0.0995 |
| L54R, E43R, S680R | 0.1716 | 0.0324 | 0.0767 | 0.0936 |
| L323R, S680R | 0.1110 | 0.0800 | 0.0857 | 0.0922 |
| D912R, T783R | 0.0413 | 0.1343 | 0.0674 | 0.0810 |
| L323R, A170R | 0.0801 | 0.0634 | 0.0990 | 0.0808 |
| D912R, S680R | 0.0458 | 0.1383 | 0.0563 | 0.0801 |
| T783R, S680R | 0.0339 | 0.1513 | 0.0397 | 0.0750 |
| D912R, E43R | 0.0460 | 0.1043 | 0.0716 | 0.0740 |
| L54R, D912R, E43R | 0.0858 | 0.0929 | 0.0302 | 0.0696 |
| D912R, T783R, E43R | 0.0426 | 0.1049 | 0.0596 | 0.0690 |
| L54R, D912R, S680R | 0.0727 | 0.0874 | 0.0207 | 0.0603 |
| D912R, T783R, S680R | 0.0327 | 0.1117 | 0.0353 | 0.0599 |
| D912R, E43R, S680R | 0.0283 | 0.0789 | 0.0359 | 0.0477 |
| Reference | 0.0105 | 0.0320 | 0.0378 | 0.0268 |

As shown in Table 18, the average indel ratio across all targets exceeded that of the reference for all variants tested (SEQ ID NO: 64). 5 Nuclease H variants resulted in indel ratios of over 0.20 when averaged across all targets, indicating that over 20% of NGS reads comprised indels. These 5 Nuclease H variants comprised the following substitution combinations: a) T783R, E43R, A170R; b) L54R, L323R, T783R; c) L323R, T783R, E43R; d) L323R, E43R, A170R; and e) L54R, T783R, A170R.

38 Nuclease H variants resulted in indel ratios between 0.10 and 0.20 when averaged across all targets, indicating between 10% and 20% of NGS reads comprised indels. 13 Nuclease H variants resulted in indel ratios of less than 0.10 when averaged across all targets, indicating less than 10% of NGS reads comprised indels.

Based on this experiment, two exemplary variants of Nuclease H, comprising the following substitutions T783R/E43R/A170R and L54R/L323R/T783R, exhibited an 8.6-fold and 8.2-fold increase in average indel activity compared to the reference Nuclease H (SEQ ID NO:64), respectively.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A CRISPR nuclease polypeptide, which comprises an amino acid sequence at least 85%, optionally 90%, identical to a reference CRISPR nuclease set forth as SEQ ID NO: 1, SEQ ID NO: 49, or SEQ ID NO: 64, wherein the CRISPR nuclease polypeptide comprises a RuvC nuclease domain and an HNH nuclease domain.

2. The CRISPR nuclease polypeptide of claim 1, which comprises an amino acid sequence at least 90% identical to a reference CRISPR nuclease set forth as SEQ ID NO: 1.

3. The CRISPR nuclease polypeptide of claim 2, wherein the CRISPR nuclease polypeptide is a variant of the reference CRISPR nuclease set forth as SEQ ID NO: 1; and wherein relative to the reference CRISPR nuclease, the CRISPR nuclease polypeptide comprises:
(i) one or more mutations in the HNH nuclease domain or in the RuvC nuclease domain that reduce or eliminate the nuclease activity thereof;
(ii) one or more arginine and/or lysine substitutions, optionally one or more arginine substitutions; or
(iii) a combination of (i) and (ii).

4. The CRISPR nuclease polypeptide of claim 3, wherein the CRISPR nuclease polypeptide comprises one or more mutations in the HNH nuclease domain at positions E425, H426, and/or N449 relative to SEQ ID NO: 1; optionally wherein the mutation is at position E425.

5. The CRISPR nuclease polypeptide of claim 4, wherein:
(a) the mutation at E425 is an amino acid substitution of E425A, E425G, E425L, or E425S;
(b) the mutation at H426 is an amino acid substitution of H426A, H426G, H426L, or H426S; and
(c) the mutation at N449 is an amino acid substitution of N449A, N449G, N449L, or N449S.

6. The CRISPR nuclease polypeptide of claim 3, wherein the CRISPR nuclease polypeptide comprises one or more mutations in the RuvC nuclease domain at positions D10, E362, and/or D487 relative to SEQ ID NO: 1.

7. The CRISPR nuclease polypeptide of claim 3, which comprises a bridge helix (BH) domain, a nucleic acid recognition (REC) domain, a phosphate lock loop (PLL) domain, a wedge (WED) domain, and a PAM-interacting (PID) domain, and wherein the one or more arginine and/or lysine substitutions, optionally arginine substitutions, are located in the BH domain, in the REC domain, in the PLL domain, in the WED domain, in the PID domain, or a combination thereof.

8. The CRISPR nuclease polypeptide of claim 3, wherein the CRISPR nuclease polypeptide contains up to 20 arginine and/or lysine substitutions, optionally arginine substitutions, relative to the reference CRISPR nuclease;
optionally wherein the CRISPR nuclease polypeptide contains up to 15 arginine and/or lysine substitutions, optionally arginine substitutions, relative to the reference CRISPR nuclease.

9. The CRISPR nuclease polypeptide of claim 8, wherein the one or more arginine and/or lysine substitutions, optionally arginine substitutions, are at positions A65, V117, A657, N691, S695, S925, and/or E715 in SEQ ID NO: 1.

10. The CRISPR nuclease polypeptide of claim 3, wherein the CRISPR nuclease polypeptide comprises an amino acid sequence at least 90% identical to SEQ ID NO: 1.

11. The CRISPR nuclease polypeptide of claim 10, wherein the CRISPR nuclease polypeptide comprises an amino acid sequence at least 95% identical to SEQ ID NO: 1.

12. The CRISPR nuclease polypeptide of claim 11, wherein the CRISPR nuclease polypeptide comprises an amino acid sequence at least 98% identical to SEQ ID NO: 1.

13. The CRISPR nuclease polypeptide of claim 2, wherein the CRISPR nuclease polypeptide comprises the mutations set forth in Table 5 or Table 6.

14. The CRISPR nuclease polypeptide of claim 2, which is a fusion polypeptide.

15. The CRISPR nuclease polypeptide of claim 14, wherein the fusion polypeptide comprises one or more nuclear localization signals (NLSs) fused to the CRISPR nuclease polypeptide at its N-terminus and/or its C-terminus, and optionally a peptide linker connecting the NLS and the CRISPR nuclease moiety in the fusion polypeptide.

16. A nucleic acid, comprising a nucleotide sequence encoding the CRISPR nuclease polypeptide of claim 2.

17. The nucleic acid of claim 16, wherein the nucleic acid is an expression vector, in which the nucleotide sequence encoding the CRISPR nuclease polypeptide is in operable linkage to a promoter.

18. The nucleic acid of claim 17, which is a messenger RNA (mRNA).

19. A host cell comprising the nucleic acid of claim 16.

20. A gene editing system, comprising:
(a) a CRISPR nuclease polypeptide or a first nucleic acid encoding the CRISPR nuclease polypeptide, wherein the CRISPR nuclease polypeptide is set forth in claim 2; and
(b) a guide RNA (gRNA), or a second nucleic acid encoding the gRNA; wherein the gRNA comprises a scaffold sequence recognizable by the CRISPR nuclease polypeptide and a spacer sequence specific to a target sequence within a genomic site of interest, the target sequence being upstream to a protospacer motif (PAM).

21. The gene editing system of claim 20, wherein the scaffold sequence comprises a nucleotide sequence at least 85% identical to SEQ ID NO: 2.

22. The gene editing system of claim 21, wherein the scaffold sequence comprises SEQ ID NO: 2.

23. The gene editing system of claim 20, wherein the scaffold sequence comprises one or more deletions, one or more nucleotide substitutions, or a combination thereof, as compared with SEQ ID NO: 2.

24. The gene editing system of claim 20, wherein the PAM is 5′-NRV-3′; optionally wherein the PAM is 5′-NAV-3′, 5′-NRR′3′, preferably 5′-RRR-3′; wherein N represents any nucleotide, R represents A or G, and V represents A, C, or G.

25. The gene editing system of claim 20, further comprise one or more lipid excipients, which are associated with the element (a) and/or element (b); optionally wherein the one or more lipid excipients form lipid nanoparticles (LNPs) associated with or encapsulating element (a) and/or element (b).

26. A gene editing method, comprising delivering the gene editing system of claim 20 to a host cell to edit a genomic site targeted by the gRNA of the gene editing system.

27. The gene editing method of claim 26, wherein the host cell is cultured in vitro.

28. The gene editing method of claim 26, wherein the host cell is located in a subject who needs the gene editing.

29. The CRISPR nuclease polypeptide of claim 1, wherein the CRISPR nuclease polypeptide comprises an amino acid sequence at least 90% identical to SEQ ID NO: 49.

30. The CRISPR nuclease polypeptide of claim 29, wherein the CRISPR nuclease polypeptide is a variant of the reference CRISPR nuclease set forth as SEQ ID NO: 49; and wherein relative to the reference CRISPR nuclease, the CRISPR nuclease polypeptide comprises:
(i) one or more mutations in the HNH nuclease domain or in the RuvC nuclease domain that reduce or eliminate the nuclease activity thereof;
(ii) one or more arginine and/or lysine substitutions, optionally one or more arginine substitutions; or
(iii) a combination of (i) and (ii).

31. The CRISPR nuclease polypeptide of claim 30, wherein the CRISPR nuclease polypeptide comprises one or more arginine and/or lysine substitutions, optionally one or more arginine substitutions, relative to the reference CRISPR nuclease.

32. The CRISPR nuclease polypeptide of claim 31, which comprises a bridge helix (BH) domain, a nucleic acid recognition (REC) domain, and a PAM-interacting (PID) domain, and wherein the one or more arginine and/or lysine substitutions are located in the BH domain, in the REC domain, in the HNH nuclease domain, in the PID domain, or a combination thereof.

33. The CRISPR nuclease polypeptide of claim 31, wherein the one or more arginine and/or lysine substitutions are located at one or more of positions T72, N76, D179, Q200, E277, H280, I322, V324, V504, W509, D844, and I845 in SEQ ID NO: 49.

34. The CRISPR nuclease polypeptide of claim 33, wherein the CRISPR nuclease polypeptide comprises arginine and/or lysine substitutions at the following positions relative to SEQ ID NO: 49:
a) W509, T72, and I845;
b) H280, T72, and I845;
c) W509 and I845;
d) H280, W509, T72, and I845;
e) D844 and W509;
f) W509 and T72;
g) D844, W509, and I845;
h) H280, W509, and I845; or
i) D844 and H280.

35. The CRISPR nuclease polypeptide of claim 34, wherein the CRISPR nuclease polypeptide comprises the following arginine substitution:
a) W509R, T72R, and I845R; or
b) H280R, T72R, and I845R.

36. The CRISPR nuclease of claim 31, wherein the CRISPR nuclease polypeptide contains up to 20 arginine and/or lysine substitutions, optionally arginine substitutions, relative to the reference CRISPR nuclease; optionally wherein the CRISPR nuclease polypeptide contains up to 15 arginine and/or lysine substitutions, optionally arginine substitutions, relative to the reference CRISPR nuclease.

37. The CRISPR nuclease of claim 31, wherein the CRISPR nuclease polypeptide comprises one or more mutations in the HNH nuclease domain at position E447, H448, and/or N471 of SEQ ID NO: 49 or in the RuvC nuclease domain at positions D25, E371, and/or D604 of SEQ ID NO:49.

38. The CRISPR nuclease of claim 37, wherein the CRISPR nuclease polypeptide comprises a mutation at position H448, which optionally is an amino acid substitution of H448A.

39. The CRISPR nuclease polypeptide of claim 29, wherein the CRISPR nuclease polypeptide comprises an amino acid sequence at least 95% identical to SEQ ID NO: 49.

40. The CRISPR nuclease polypeptide of claim 39, wherein the CRISPR nuclease polypeptide comprises an amino acid sequence at least 98% identical to SEQ ID NO: 49.

41. The CRISPR nuclease polypeptide of claim 29, wherein the CRISPR nuclease polypeptide is in a fusion polypeptide format, which further comprises one or more functional fragments.

42. The CRISPR nuclease polypeptide of claim 41, wherein the fusion polypeptide comprises one or more nuclear localization signals (NLSs) fused to the CRISPR nuclease polypeptide at its N-terminus and/or its C-terminus, and optionally a peptide linker connecting the NLS and the CRISPR nuclease moiety in the fusion polypeptide.

43. A nucleic acid, comprising a nucleotide sequence encoding the CRISPR nuclease of claim 29.

44. The nucleic acid of claim 43, wherein the nucleic acid is an expression vector, in which the nucleotide sequence encoding the CRISPR nuclease is in operable linkage to a promoter.

45. The nucleic acid of claim 43, which is a messenger RNA (mRNA).

46. A host cell comprising the nucleic acid of claim 43.

47. A gene editing system, comprising:
(a) a CRISPR nuclease polypeptide or a first nucleic acid encoding the CRISPR nuclease; wherein the CRISPR nuclease polypeptide is set forth in claim 29; and
(b) a guide RNA (gRNA), or a second nucleic acid encoding the gRNA, wherein the gRNA comprises a scaffold sequence recognizable by the CRISPR nuclease and a spacer sequence specific to a target sequence within a genomic site of interest, the target sequence being upstream to a protospacer motif (PAM).

48. The gene editing system of claim 47, wherein the scaffold sequence comprises a nucleotide sequence at least 85% identical to SEQ ID NO: 63.

49. The gene editing system of claim 48, wherein the scaffold sequence is a fragment of SEQ ID NO: 63.

50. The gene editing system of claim 47, wherein the scaffold sequence comprises one or more deletions, one or more nucleotide substitutions, or a combination thereof, as compared with SEQ ID NO: 63.

51. The gene editing system of claim 47, wherein the target sequence is adjacent to a protospacer adjacent motif of 5′-NRNC-3′, optionally 5′-NRCC-3′ or 5′-ARCC-3′, or 5′-NRYM-3′, in which N represents any nucleotide, R represents A or G, Y represents C or T, and M represents A or C.

52. The gene editing system of claim 47, further comprise one or more lipid excipients, which are associated with the element (a) and/or element (b); optionally wherein the one or more lipid excipients form lipid nanoparticles (LNPs) associated with or encapsulating element (a) and/or element (b).

53. A gene editing method, comprising delivering the gene editing system of claim 47 to a host cell to edit a genomic site targeted by the gRNA of the gene editing system.

54. A CRISPR nuclease polypeptide, which comprises an amino acid sequence at least 90% identical to SEQ ID NO: 64.

55. The CRISPR nuclease polypeptide of claim 54, wherein the CRISPR nuclease polypeptide is a variant of the reference CRISPR nuclease set forth as SEQ ID NO: 64; and wherein relative to the reference CRISPR nuclease, the CRISPR nuclease polypeptide comprises:
(i) one or more mutations in the HNH nuclease domain or in the RuvC nuclease domain that reduce or eliminate the nuclease activity thereof;
(ii) one or more arginine and/or lysine substitutions, optionally one or more arginine substitutions; or
(iii) a combination of (i) and (ii).

56. The CRISPR nuclease polypeptide of claim 55, wherein the at least one mutation comprises one or more arginine substitutions, lysine substitutions, or a combination thereof, relative to the reference CRISPR nuclease.

57. The CRISPR nuclease polypeptide of claim 56, wherein the at least one mutation is located in a bridge helix (BH) domain, in a nucleic acid recognition (REC) domain, in a phosphate lock loop (PLL) domain, in a wedge (WED) domain, in a PAM-interacting (PID) domain, in a nuclease domain, or a combination thereof.

58. The CRISPR nuclease polypeptide of claim 56, wherein the CRISPR nuclease polypeptide contains up to 20 arginine and/or lysine substitutions, optionally up to arginine substitutions, relative to the reference CRISPR nuclease.

59. The CRISPR nuclease polypeptide of claim 58, wherein the CRISPR nuclease polypeptide contains up to 15 arginine and/or lysine substitutions, optionally up to 15 arginine substitutions, relative to the reference CRISPR nuclease.

60. The CRISPR nuclease polypeptide of claim 55, wherein the one or more arginine and/or lysine substitutions are at position E43, L54, A170, L323, S680, T783, and/or D912 of SEQ ID NO: 64; optionally wherein the CRISPR nuclease polypeptide comprises one or more arginine substitutions E43R, L54R, A170R, L323R, S680R, T783R, and/or D912R.

61. The CRISPR nuclease polypeptide of claim 60, which comprises arginine and/or lysine substitutions at positions:
(a) E43, A170, and T783, or
(b) L54, L323, and T783;
optionally wherein the CRISPR nuclease polypeptide comprises arginine substitutions (a) E43R, A170R, and T783R; or (b) L54R, L323R, and T783R.

62. The CRISPR nuclease polypeptide of claim 55, wherein the CRISPR nuclease polypeptide comprises the one or more nickase mutations of (b), which optionally are located at D7, R329, H338, E356, E419, H420, N443, and/or D580 of SEQ ID NO: 64.

63. The CRISPR nuclease polypeptide of claim 54, wherein the CRISPR nuclease polypeptide comprises an amino acid sequence at least 95% identical to SEQ ID NO: 64; optionally wherein the CRISPR nuclease polypeptide comprises an amino acid sequence at least 98% identical to SEQ ID NO: 64.

64. The CRISPR nuclease polypeptide of claim 54, wherein the CRISPR nuclease polypeptide is in a fusion polypeptide format, which further comprises one or more functional fragments.

65. The CRISPR nuclease polypeptide of claim 64, wherein the fusion polypeptide comprises one or more nuclear localization signals (NLSs) fused to the CRISPR nuclease polypeptide at its N-terminus and/or its C-terminus, and optionally a peptide linker connecting the NLS and the CRISPR nuclease moiety in the fusion polypeptide.

66. A nucleic acid, comprising a nucleotide sequence encoding the CRISPR nuclease polypeptide of claim 55.

67. The nucleic acid of claim 66, wherein the nucleic acid is an expression vector, in which the nucleotide sequence encoding the CRISPR nuclease is in operable linkage to a promoter.

68. The nucleic acid of claim 66, which is a messenger RNA (mRNA).

69. A host cell comprising the nucleic acid of claim 66.

70. A gene editing system, comprising:
(a) a CRISPR nuclease polypeptide or a first nucleic acid encoding the CRISPR nuclease, wherein the CRISPR nuclease polypeptide is set forth in claim 54; and
(b) a guide RNA (gRNA), or a second nucleic acid encoding the gRNA, wherein the gRNA comprises a scaffold sequence recognizable by the CRISPR nuclease polypeptide and a spacer sequence specific to a target sequence within a genomic site of interest, the target sequence being upstream to a protospacer motif (PAM).

71. The gene editing system of claim 70, wherein the scaffold sequence comprises a nucleotide sequence at least 85% identical to SEQ ID NO: 77.

72. The gene editing system of claim 71, wherein the scaffold sequence is a fragment of SEQ ID NO: 77.

73. The gene editing system of claim 70, wherein the scaffold sequence comprises one or more deletions, one or more nucleotide substitutions, or a combination thereof, as compared with SEQ ID NO: 77.

74. The gene editing system of claim 70, wherein the target sequence is adjacent to a protospacer adjacent motif of 5′-NRM-3′, optionally 5′-NRC-3′, or 5′-NRMY-3′, optionally 5′-NRCY-3′, in which N represents any nucleotide, R represents A or G, M represents A or C, and Y represents C.

75. The gene editing system of claim 70, further comprise one or more lipid excipients, which are associated with the element (a) and/or element (b); optionally wherein the one or more lipid excipients form lipid nanoparticles (LNPs) associated with or encapsulating element (a) and/or element (b).

76. A gene editing method, comprising delivering the gene editing system of claim 70 to a host cell to edit a genomic site targeted by the gRNA of the gene editing system.


SEQUENCE LISTING (101 sequences)

SEQ ID NO: 1 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 2 - RNA, 174 nt, synthetic construct
  source 1..174: mol_type = other RNA
gtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 3 - protein, 18 aa, synthetic construct
KRTADGSEFESPKKKRKV

SEQ ID NO: 4 - protein, 16 aa, synthetic construct
KRPAATKKAGQAKKKK

SEQ ID NO: 5 - protein, 33 aa, synthetic construct
SGGSSGGSSGSETPGTSESATPESSGGSSGGSS

SEQ ID NO: 6 - DNA, 54 bp, synthetic construct
  source 1..54: mol_type = other DNA
aaacggacagccgacggaagcgagttcgagtcaccaaagaagaagcggaaagtc

SEQ ID NO: 7 - DNA, 2865 bp, synthetic construct
  source 1..2865: mol_type = other DNA
atgagctcaacacacacactggccatcgatttcggcagcaagaacatcggcatcgccctggtgcggaatcaggacagcctgaacacccctctgttcgctggcacagccctgtacgacagattcacactatccaagaaggtggaacctcgggtgcagatgcgacggatgagaagaaccaagaagacaagaaaggccaggctggcccacctggaggccggcctgctctctcacggcctggagcctgacgttgtggtgtggctggtgaacttctgcagaagaagaggcttcaaaagcctgtttgacgagggccgggaaccccggggcgaaaagaaggaggaagaggctgaggtttttcggtttagcagagaagaattcttctccgccctggagaacgagctgctggaaagactaccagaggaaaagctgatccctgtgctggacctgtgcgagcatggcctgaatagagatggcgaccgtttcaaggaggtgcgtcttatcagaatcgacaaccggggcgctagcagatgcgcctgggacggctgtaacagcgtgacccctagaagagataatgccctgagagacgcccttgcccagttcgtgttcaccgtgtacgcccagaaggtgagatctgatcccgacctgtacgagaaaatccagaggatgctggaccagctagcccagctgggcaagagactgagacatgtgggcggatctgatcctaagaaggaacgccaggtgctgctgaagcggatccgggaagagctgaaactcctgaagttcctgagcggcctggcccaagatctgccagaggagacaatcgagcctaaagaggcctggacccacatcagaagaaacctgatgaacctgatcgagcagtccggcggcagaaacagattttgcagacaccacagcgctcaatacgtgtaccacctcatcgaaggcaagcctatccccttcaagagatccctgaccgagagcgacatgaccagcagacgggaagaaatcctgttccagaaactgtggcgatatatcgaggccagagtgctgccactggcccctgccggaattgacagggtggtcgtggaaagaacagccttcgacatgctggccggatctcggaaacagaggcagaaagtggccgacgactacctggaagaaatgtaccagcagggcccccggtacggcttcaaggatgacctggagatgctgagaaaggaatttaacggcctttgcgcctactgcggtaaacctagcgaaaccatcgtggagagagagcacatcctgccgaaggccaacttcttcttcgacagctacctgaacatggtgcccgcgtgtcctgattgtaacagaatgtggaaggccaaggccagccctggagcctctgggctgcggattaacgagtctgcttaccaggcttacagcgattacctcgctgacaaattcaagaccagaccaccacacctgttccacaccatcaagaaaggcatcctgaagctgatgacacaaaaggacagggtgctggaggccgagagactgctgggcatgatcgccgaaaacctgggaagagtggtggaaagccagcggggacctagacctttggctcgctacctgtgcgagaaactgagacagcggtacgaccgggtgacccagatcgcctttagaagcggacggcacaccgccatctggagacaggctgcttatcctgatttcgacaaggccaaggaaaaggtcagcggcggcaccatcaatcacgccctcgacgctatgatcctggcctgtgatctgcccgacgtgaccgccctggaggccagaaacctgcctccttggatcatcagcgcgtgggtggacaaggtgagaagcgccgcacctcaggccggccaggagggaatcccagctatccctcatcctgatttcgccgtgcccgagttcgagaaggtcctgcagggcaactttatcgagacagacctggctaagctgaactggaacagaaagcactctagagtgcagcgacaggacgcctatggctggtgcggcctggaggacgtgcccgccaagagaatcgccgctgcaaatctggctgctagtctgagaaacgccgacaaggagaagacccccgaggacagacagaaagaggtgaagaaggaggtggtggtggtggttcaccctagactgcgggctgttctggaggccgccaataccggcgaaagacctggtgaaagctccgccgccgccctgattagctggctgagaaaggccgtgaagggaagcctgaaagatgctcgcctcagcagccaccccgccgaccaggccagagctaaagccctgagagacttcgccgacggcacgggcgaggccatccctgccgtgatcggcgtgagaatccggtacccctggctgaaggccaacatggacctgcaccgggtggaacggcagacaggccagatcgtgcacagatatgtggccgaccccgctaaccaggccctgatcgttgcctacctgggcagcaacggcaaggtgagaagagacaagcccatgaccctggagctgcgccaatctggagccgtggtgcctggcttcaaggcactgtctgaagtccccgccggccctctgcagggcagagccctgggccaacctcggcctgatcctggagagtggcaggccgctctgcacgcctacctgcagtccgccgaagtggccgagtacgccatggtttcccagggctgcgtggtgcactacgaggatggctctgagcggtacataagaaatttcagcagcagctacggctttaaaaagagcctgttcaagggcatggtcggagtgcggcggtctccactgagcgggagagtgatccccaacatcagaatt

SEQ ID NO: 8 - DNA, 48 bp, synthetic construct
  source 1..48: mol_type = other DNA
aaaaggccggcggccacgaaaaaggccggccaggcaaaaaagaaaaag

SEQ ID NO: 9 - DNA, 99 bp, synthetic construct
  source 1..99: mol_type = other DNA
tctggaggatctagcggaggatcctctggcagcgagacaccaggaacaagcgagtcagcaacaccagagagcagtggcggcagcagcggcggcagcagc

SEQ ID NO: 10 - protein, 1022 aa, synthetic construct
MKRTADGSEFESPKKKRKVSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRISGGSSGGSSGSETPGTSESATPESSGGSSGGSSKRPAATKKAGQAKKKK

SEQ ID NO: 11 - DNA, 20 bp, synthetic construct
  source 1..20: mol_type = other DNA
ggtgcgtcctaggtgttcac

SEQ ID NO: 12 - RNA, 194 nt, synthetic construct
  source 1..194: mol_type = other RNA
ggtgcgtcctaggtgttcacgtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 13 - DNA, 20 bp, synthetic construct
  source 1..20: mol_type = other DNA
ttaaagcgactccaatgcgg

SEQ ID NO: 14 - RNA, 194 nt, synthetic construct
  source 1..194: mol_type = other RNA
ttaaagcgactccaatgcgggtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 15 - DNA, 20 bp, synthetic construct
  source 1..20: mol_type = other DNA
ataggccagaggcagcttcc

SEQ ID NO: 16 - RNA, 194 nt, synthetic construct
  source 1..194: mol_type = other RNA
ataggccagaggcagcttccgtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 17 - DNA, 20 bp, synthetic construct
  source 1..20: mol_type = other DNA
taaactacagtggtgcctgg

SEQ ID NO: 18 - RNA, 194 nt, synthetic construct
  source 1..194: mol_type = other RNA
taaactacagtggtgcctgggtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 19 - DNA, 20 bp, synthetic construct
  source 1..20: mol_type = other DNA
cccagtatgggcaaaggaaa

SEQ ID NO: 20 - RNA, 194 nt, synthetic construct
  source 1..194: mol_type = other RNA
aggctctggagagatttgtggtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 21 - DNA, 20 bp, synthetic construct
  source 1..20: mol_type = other DNA
cccagtatgggcaaaggaaa

SEQ ID NO: 22 - RNA, 194 nt, synthetic construct
  source 1..194: mol_type = other RNA
cccagtatgggcaaaggaaagtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctg

SEQ ID NO: 23 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaacacattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 24 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgcgcacattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 25 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVERAHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 26 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaagcgattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 27 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREAILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 28 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaactgattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 29 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVERELILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 30 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaacacattctgccgaaagcgaacttcttcttcgacagctatctgaacatggttccggcgtgcccggactgtgcgcgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 31 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREHILPKANFFFDSYLNMVPACPDCARMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 32 - DNA, 2865 bp, synthetic construct
  source 1..2865: mol_type = other DNA
atgagttcaacacacactctagctatagcgttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaacacattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 33 - protein, 955 aa, synthetic construct
MSSTHTLAIAFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 34 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggcgcgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaacacattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggacgccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 35 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVARTAFDMLAGSRKQRQKVADDYLEEMYQQGPRYGFKDDLEMLRKEFNGLCAYCGKPSETIVEREHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALDAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 36 - DNA, 2865 bp, synthetic construct
  source 1..2865: mol_type = other DNA
atgagttcaacacacactctagctatagatttcggtagcaaaaacattggcatcgctctggtgcgcaaccaggacagccttaacaccccgctgttcgcgggtaccgcactgtacgaccggtttaccctgtcgaaaaaagtggagccacgtgttcagatgcgacgcatgcgtcggacgaaaaagacccgtaaggctagattggcacatctggaagcgggattgctgagccacggtctggagccggacgtcgtcgtttggctggttaatttctgcaggaggagaggcttcaagagcctgttcgatgaaggtcgtgaaccacgtggtgaaaaaaaagaggaggaggcggaagtgttccgttttagccgtgaggaattcttcagcgcgttagagaacgaactgctggagcgcctgccggaagagaagcttatcccggttctagacctctgcgaacacgggttgaaccgtgatggcgatcgctttaaggaggttagattgatccgtattgataaccgtggtgcttcccgttgtgcatgggatggctgcaatagcgtcaccccgcgtagagacaatgcactacgtgatgcactggcgcagttcgtttttacggtgtatgctcaaaaggtgcgctccgatccggatctgtacgaaaaaattcagcgcatgttagatcaactggcccagctaggtaagcgtctgcgccacgttggtggttccgacccgaaaaaagagcgtcaagttctgctgaagcgcatccgtgaagagctgaagttgctcaaattcctgtctggcttagcccaggacctgccggaagaaacgatcgaaccgaaagaggcctggacccatattcgtcgtaacctgatgaatctgattgagcagagcgggggccgtaaccgtttttgccgtcatcatagcgcgcaatatgtttaccatctgatcgagggcaaaccgatcccgtttaagagatctcttaccgaaagcgacatgacgagccgtcgtgaagagattctgtttcaaaaactgtggcgttatatcgaagcccgtgtgctcccgctggctccagcgggcatcgatagagtcgtggtggaacgaaccgcgtttgacatgctggcgggtagccgtaaacagcgtcaaaaagttgcggacgactatttggaggaaatgtatcagcagggtccgcgttacggctttaaggacgatctggaaatgctgcgcaaggaattcaacggtttgtgcgcatactgcggtaagccgagcgagaccattgttgaacgcgaacacattctgccgaaagcgaacttcttcttcgacagctatctgaacatggttccggcgtgcccggactgtaaccgtatgtggaaagccaaggcgtccccgggtgcaagcggtttacgtatcaacgagagtgcataccaagcttacagcgattacctggctgacaagtttaagacccgtccaccgcatttgttccacacgattaaaaaaggaattctgaagctgatgacccagaaagatcgcgtgttggaagcggagaggttgctgggcatgatcgctgaaaacttgggccgtgttgtggagagccagcgtggcccgcgtccgctggctcgctacctgtgtgaaaagctccgtcaacgttacgatcgtgtgacccagatcgcatttcgttctggtagacacacggcgatctggcgtcaagcggcttacccggactttgataaagcgaaagagaaagtgtcgggtggaaccatcaatcatgcattggcggccatgattctggcctgcgatttgccagacgttaccgcccttgaggcgcgcaacctgccaccgtggatcatttctgcgtgggttgataaagttcgttccgcggcaccgcaggctggccaagagggcattccggccatcccgcatccggactttgcggtaccggaatttgaaaaagttttacaaggtaacttcatcgaaaccgacttggctaagctgaactggaaccgcaagcacagccgtgtgcagcgccaagatgcatacggctggtgtggtctggaggacgtgccggctaagcgcatcgccgcggcgaatctggcggcgtccctccgcaacgcagataaggagaaaactccggaggaccgccaaaaggaggtgaaaaaagaagtggttgtcgttgtgcacccgcgtctacgtgctgttttggaggcggcgaataccggtgagcgtccgggcgagtctagcgcggctgcgctcatcagctggctgagaaaggccgtaaaaggttctctcaaggatgcgcgcctgtcatctcacccggcagaccaggcgcgtgcaaaggccctcagagattttgccgacggtactggtgaggccataccggctgtgattggcgttcgtattcgctacccgtggctgaaagcgaacatggatttgcaccgcgtagagcgccaaaccggtcaaattgtgcaccgctacgtggctgatccggcgaaccaggccctcatcgtcgcgtacctgggcagcaatggtaaggtgcgtcgagataaacctatgactttggaactgaggcagtcgggtgccgtggtgcccggcttcaaggcgctcagcgaagttccggcgggccctctgcaaggtcgtgcattgggccagccgcgtccggacccgggcgagtggcaggcggcgctgcacgcgtatctgcaatccgcggaggtggccgagtatgcaatggtttcccagggttgcgtcgtgcactatgaggacggttcggaacgttatatccgtaatttctctagctcatatggttttaagaagagcctgttcaagggcatggtcggtgtgagacgctccccgctgtccggccgcgttattccgaatatccgcatc

SEQ ID NO: 37 - protein, 955 aa, synthetic construct
MSSTHTLAIDFGSKNIGIALVRNQDSLNTPLFAGTALYDRFTLSKKVEPRVQMRRMRRTKKTRKARLAHLEAGLLSHGLEPDVVVWLVNFCRRRGFKSLFDEGREPRGEKKEEEAEVFRFSREEFFSALENELLERLPEEKLIPVLDLCEHGLNRDGDRFKEVRLIRIDNRGASRCAWDGCNSVTPRRDNALRDALAQFVFTVYAQKVRSDPDLYEKIQRMLDQLAQLGKRLRHVGGSDPKKERQVLLKRIREELKLLKFLSGLAQDLPEETIEPKEAWTHIRRNLMNLIEQSGGRNRFCRHHSAQYVYHLIEGKPIPFKRSLTESDMTSRREEILFQKLWRYIEARVLPLAPAGIDRVVVERTAFDMLAGSRKQRQKVADDYLEEMYQQGPRYGFKDDLEMLRKEFNGLCAYCGKPSETIVEREHILPKANFFFDSYLNMVPACPDCNRMWKAKASPGASGLRINESAYQAYSDYLADKFKTRPPHLFHTIKKGILKLMTQKDRVLEAERLLGMIAENLGRVVESQRGPRPLARYLCEKLRQRYDRVTQIAFRSGRHTAIWRQAAYPDFDKAKEKVSGGTINHALAAMILACDLPDVTALEARNLPPWIISAWVDKVRSAAPQAGQEGIPAIPHPDFAVPEFEKVLQGNFIETDLAKLNWNRKHSRVQRQDAYGWCGLEDVPAKRIAAANLAASLRNADKEKTPEDRQKEVKKEVVVVVHPRLRAVLEAANTGERPGESSAAALISWLRKAVKGSLKDARLSSHPADQARAKALRDFADGTGEAIPAVIGVRIRYPWLKANMDLHRVERQTGQIVHRYVADPANQALIVAYLGSNGKVRRDKPMTLELRQSGAVVPGFKALSEVPAGPLQGRALGQPRPDPGEWQAALHAYLQSAEVAEYAMVSQGCVVHYEDGSERYIRNFSSSYGFKKSLFKGMVGVRRSPLSGRVIPNIRI

SEQ ID NO: 38 - RNA, 20 nt, synthetic construct
  source 1..20: mol_type = other RNA
cccagtatgggcaaaggaaa

SEQ ID NO: 39 - RNA, 28 nt, synthetic construct
  source 1..28: mol_type = other RNA
ggctcaccttcgggtgggcctttctgcg

SEQ ID NO: 40 - RNA, 223 nt, synthetic construct
  source 1..223: mol_type = other RNA
gcccagtatgggcaaaggaaagtttcagctaccctgagtttctcaactccgttgagacttcctcaggttcaagctgaaaagagcatctgtccggaaggaccactccgggttagggcagacccggctcttgggcttccttggtctttgactctggaggaaaccttccggcgtgtcgcaagacaagatgtgagcctgggctcaccttcgggtgggcctttctgcg

SEQ ID NO: 41 - DNA, 10 bp, synthetic construct
  source 1..10: mol_type = other DNA
ttccttggtc

SEQ ID NO: 42 - DNA, 10 bp, synthetic construct
  source 1..10: mol_type = other DNA
gaggtacaga

SEQ ID NO: 43 - DNA, 100 bp, synthetic construct
  source 1..100: mol_type = other DNA
gctcaaagctttagactggaggtaccgtagtactatgagcttaatcgcgctgagggtgatttgggtgttcgtctcatatcttcgattatcggacagtagt

SEQ ID NO: 44 - DNA, 200 bp, synthetic construct
  source 1..200: mol_type = other DNA
agtagtatcggaccggttggcgaaccacagcgtataacgagacatggactcgcattccctaccttaagcgggcggttttggagctcggaattcatgtaaactcgcccgtttaacataatctagatggatcttgcgcgcaccgcccgaacaccactcgccacaaaaaaccgccggaacgtccaaaagtacgggttttgctg

SEQ ID NO: 45 - DNA, 340 bp, synthetic construct
  source 1..340: mol_type = other DNA
gctcaaagctttagactggaggtaccgtagtactatgagcttaatcgcgctgagggtgatttgggtgttcgtctcatatcttcgattatcggacagtagtttccttggtccccagtatgggcaaaggaaagaggtacagaagtagtatcggaccggttggcgaaccacagcgtataacgagacatggactcgcattccctaccttaagcgggcggttttggagctcggaattcatgtaaactcgcccgtttaacataatctagatggatcttgcgcgcaccgcccgaacaccactcgccacaaaaaaccgccggaacgtccaaaagtacgggttttgctg

SEQ ID NO: 46 - DNA, 27 bp, synthetic construct
  source 1..27: mol_type = other DNA
gctcaaagctttagactggaggtaccg

SEQ ID NO: 47 - DNA, 24 bp, synthetic construct
  source 1..24: mol_type = other DNA
cagcaaaacccgtacttttggacg

SEQ ID NO: 48 - DNA, 3066 bp, synthetic construct
  source 1..3066: mol_type = other DNA
atgaaacggacagccgacggaagcgagttcgagtcaccaaagaagaagcggaaagtcagctcaacacacacactggccatcgatttcggcagcaagaacatcggcatcgccctggtgcggaatcaggacagcctgaacacccctctgttcgctggcacagccctgtacgacagattcacactatccaagaaggtggaacctcgggtgcagatgcgacggatgagaagaaccaagaagacaagaaaggccaggctggcccacctggaggccggcctgctctctcacggcctggagcctgacgttgtggtgtggctggtgaacttctgcagaagaagaggcttcaaaagcctgtttgacgagggccgggaaccccggggcgaaaagaaggaggaagaggctgaggtttttcggtttagcagagaagaattcttctccgccctggagaacgagctgctggaaagactaccagaggaaaagctgatccctgtgctggacctgtgcgagcatggcctgaatagagatggcgaccgtttcaaggaggtgcgtcttatcagaatcgacaaccggggcgctagcagatgcgcctgggacggctgtaacagcgtgacccctagaagagataatgccctgagagacgcccttgcccagttcgtgttcaccgtgtacgcccagaaggtgagatctgatcccgacctgtacgagaaaatccagaggatgctggaccagctagcccagctgggcaagagactgagacatgtgggcggatctgatcctaagaaggaacgccaggtgctgctgaagcggatccgggaagagctgaaactcctgaagttcctgagcggcctggcccaagatctgccagaggagacaatcgagcctaaagaggcctggacccacatcagaagaaacctgatgaacctgatcgagcagtccggcggcagaaacagattttgcagacaccacagcgctcaatacgtgtaccacctcatcgaaggcaagcctatccccttcaagagatccctgaccgagagcgacatgaccagcagacgggaagaaatcctgttccagaaactgtggcgatatatcgaggccagagtgctgccactggcccctgccggaattgacagggtggtcgtggaaagaacagccttcgacatgctggccggatctcggaaacagaggcagaaagtggccgacgactacctggaagaaatgtaccagcagggcccccggtacggcttcaaggatgacctggagatgctgagaaaggaatttaacggcctttgcgcctactgcggtaaacctagcgaaaccatcgtggagagagagcacatcctgccgaaggccaacttcttcttcgacagctacctgaacatggtgcccgcgtgtcctgattgtaacagaatgtggaaggccaaggccagccctggagcctctgggctgcggattaacgagtctgcttaccaggcttacagcgattacctcgctgacaaattcaagaccagaccaccacacctgttccacaccatcaagaaaggcatcctgaagctgatgacacaaaaggacagggtgctggaggccgagagactgctgggcatgatcgccgaaaacctgggaagagtggtggaaagccagcggggacctagacctttggctcgctacctgtgcgagaaactgagacagcggtacgaccgggtgacccagatcgcctttagaagcggacggcacaccgccatctggagacaggctgcttatcctgatttcgacaaggccaaggaaaaggtcagcggcggcaccatcaatcacgccctcgacgctatgatcctggcctgtgatctgcccgacgtgaccgccctggaggccagaaacctgcctccttggatcatcagcgcgtgggtggacaaggtgagaagcgccgcacctcaggccggccaggagggaatcccagctatccctcatcctgatttcgccgtgcccgagttcgagaaggtcctgcagggcaactttatcgagacagacctggctaagctgaactggaacagaaagcactctagagtgcagcgacaggacgcctatggctggtgcggcctggaggacgtgcccgccaagagaatcgccgctgcaaatctggctgctagtctgagaaacgccgacaaggagaagacccccgaggacagacagaaagaggtgaagaaggaggtggtggtggtggttcaccctagactgcgggctgttctggaggccgccaataccggcgaaagacctggtgaaagctccgccgccgccctgattagctggctgagaaaggccgtgaagggaagcctgaaagatgctcgcctcagcagccaccccgccgaccaggccagagctaaagccctgagagacttcgccgacggcacgggcgaggccatccctgccgtgatcggcgtgagaatccggtacccctggctgaaggccaacatggacctgcaccgggtggaacggcagacaggccagatcgtgcacagatatgtggccgaccccgctaaccaggccctgatcgttgcctacctgggcagcaacggcaaggtgagaagagacaagcccatgaccctggagctgcgccaatctggagccgtggtgcctggcttcaaggcactgtctgaagtccccgccggccctctgcagggcagagccctgggccaacctcggcctgatcctggagagtggcaggccgctctgcacgcctacctgcagtccgccgaagtggccgagtacgccatggtttcccagggctgcgtggtgcactacgaggatggctctgagcggtacataagaaatttcagcagcagctacggctttaaaaagagcctgttcaagggcatggtcggagtgcggcggtctccactgagcgggagagtgatccccaacatcagaatttctggaggatctagcggaggatcctctggcagcgagacaccaggaacaagcgagtcagcaacaccagagagcagtggcggcagcagcggcggcagcagcaaaaggccggcggccacgaaaaaggccggccaggcaaaaaagaaaaag

SEQ ID NO: 49 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 50 - protein, 1059 aa, synthetic construct
MKRTADGSEFESPKKKRKVDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITKSGGSSGGSSGSETPGTSESATPESSGGSSGGSSKRPAATKKAGQAKKKK

SEQ ID NO: 51 - DNA, 20 bp, synthetic construct
  source 1..20: mol_type = other DNA
ggatccctagctgggccctg

SEQ ID NO: 52 - RNA, 229 nt, synthetic construct
  source 1..229: mol_type = other RNA
ggatccctagctgggccctggttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 53 - DNA, 20 bp, synthetic construct
  source 1..20: mol_type = other DNA
ggctataatccatgctgacc

SEQ ID NO: 54 - RNA, 229 nt, synthetic construct
  source 1..229: mol_type = other RNA
ggctataatccatgctgaccgttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 55 - DNA, 20 bp, synthetic construct
  source 1..20: mol_type = other DNA
atgactccaggcctccccaa

SEQ ID NO: 56 - RNA, 229 nt, synthetic construct
  source 1..229: mol_type = other RNA
atgactccaggcctccccaagttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 57 - DNA, 20 bp, synthetic construct
  source 1..20: mol_type = other DNA
tccaaagctttccttctttg

SEQ ID NO: 58 - RNA, 229 nt, synthetic construct
  source 1..229: mol_type = other RNA
tccaaagctttccttctttggttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 59 - DNA, 20 bp, synthetic construct
  source 1..20: mol_type = other DNA
cctccaccagtcatggtgac

SEQ ID NO: 60 - RNA, 229 nt, synthetic construct
  source 1..229: mol_type = other RNA
cctccaccagtcatggtgacgttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 61 - DNA, 20 bp, synthetic construct
  source 1..20: mol_type = other DNA
ctctacacaaatctctccag

SEQ ID NO: 62 - RNA, 229 nt, synthetic construct
  source 1..229: mol_type = other RNA
ctctacacaaatctctccaggttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 63 - RNA, 209 nt, synthetic construct
  source 1..209: mol_type = other RNA
gttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaa

SEQ ID NO: 64 - protein, 949 aa, synthetic construct
MITLGIDYGAAYVGIALVRNHENGHDEPLFAATMVLDARRLKEKVENRAALRRLRRTRKTKKRRLRELKDKLLRLGLAPETVSQIVNFCERRGYKSLFGQEGDPDDKSRDELTYRFTREEFFQTLEGHLSRLLLSPEAKTAALAICERVLNRAGDRRLEVRPLRIENRGASRCAWEGCDRVTPRRSNAIEDAITQQLVTYFQSTLKQHQDLLDTLKVATAEMGRISHQLRSAVEKGLAPEVKALRKRARDVLKRLRVEFTEPEAAEENDEKAWKYVKEGIMNILEKARGRNAYCREHSAEYVQTVLAGRAVPFKKTIAESDILSRREQIAFSKLWRYIEARVLPLAPEGIDRIVVERTAFDVLAGNRRKIRTTSEKRVEEMYQQGPRFGFKDDGAMLREEFGGLCAYCGKPSPDLIEREHVLPRRDFFFDSYLNILPACPDCNRQKGRSRPGSAALHISEEAYANYDRYLKRIAGQRPVHFLHYEKKGILNLMRDPKRAWDAERYLGLLANNFAAIAQSQRGPRPFARLLFSKLAARQNKPPTISFFSGRHTAMYRTIAYPGFEKHKDKAEGGTMNHALDAVLLACELPNASVLEARGINVQDLETWRRRVRSRAPVPGKDGVPDIPLYEWCVPGFEEVGLNGYVEVQMAAMNWNQKDSSTHKQDPYGWSEESNKPTKRSSAVELYEKLMNEKADEKVKNIVQTIHHPSLRSAMSQGLKARPPGPAVAEAMKEWLRKSVRNSLGKSSCTSHPGDLRRRTDLQNFAENHDAKIPRVIGVKRLDTGVQGKIDLERLDPMNGRTLHRYMSDPVNREVLLAYRVGSNGQPDFLKPFTVKVKQNLALKTDLRTFQPIPPDLACGVTWGSAASLGDGWSSKLETYLSERGFCSYARIKPCCVVCYQDGSQLFIRNFDDNKDFKPSKILKNVVGVKRTPFALRMVPLKVLTPQRPK

SEQ ID NO: 65 - protein, 1016 aa, synthetic construct
MKRTADGSEFESPKKKRKVITLGIDYGAAYVGIALVRNHENGHDEPLFAATMVLDARRLKEKVENRAALRRLRRTRKTKKRRLRELKDKLLRLGLAPETVSQIVNFCERRGYKSLFGQEGDPDDKSRDELTYRFTREEFFQTLEGHLSRLLLSPEAKTAALAICERVLNRAGDRRLEVRPLRIENRGASRCAWEGCDRVTPRRSNAIEDAITQQLVTYFQSTLKQHQDLLDTLKVATAEMGRISHQLRSAVEKGLAPEVKALRKRARDVLKRLRVEFTEPEAAEENDEKAWKYVKEGIMNILEKARGRNAYCREHSAEYVQTVLAGRAVPFKKTIAESDILSRREQIAFSKLWRYIEARVLPLAPEGIDRIVVERTAFDVLAGNRRKIRTTSEKRVEEMYQQGPRFGFKDDGAMLREEFGGLCAYCGKPSPDLIEREHVLPRRDFFFDSYLNILPACPDCNRQKGRSRPGSAALHISEEAYANYDRYLKRIAGQRPVHFLHYEKKGILNLMRDPKRAWDAERYLGLLANNFAAIAQSQRGPRPFARLLFSKLAARQNKPPTISFFSGRHTAMYRTIAYPGFEKHKDKAEGGTMNHALDAVLLACELPNASVLEARGINVQDLETWRRRVRSRAPVPGKDGVPDIPLYEWCVPGFEEVGLNGYVEVQMAAMNWNQKDSSTHKQDPYGWSEESNKPTKRSSAVELYEKLMNEKADEKVKNIVQTIHHPSLRSAMSQGLKARPPGPAVAEAMKEWLRKSVRNSLGKSSCTSHPGDLRRRTDLQNFAENHDAKIPRVIGVKRLDTGVQGKIDLERLDPMNGRTLHRYMSDPVNREVLLAYRVGSNGQPDFLKPFTVKVKQNLALKTDLRTFQPIPPDLACGVTWGSAASLGDGWSSKLETYLSERGFCSYARIKPCCVVCYQDGSQLFIRNFDDNKDFKPSKILKNVVGVKRTPFALRMVPLKVLTPQRPKSGGSSGGSSGSETPGTSESATPESSGGSSGGSSKRPAATKKAGQAKKKK

SEQ ID NO: 66 - DNA, 20 bp, synthetic construct
  source 1..20: mol_type = other DNA
acgacctggtgaacacctag

SEQ ID NO: 67 - RNA, 254 nt, synthetic construct
  source 1..254: mol_type = other RNA
acgacctggtgaacacctaggtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 68 - RNA, 254 nt, synthetic construct
  source 1..254: mol_type = other RNA
ggctataatccatgctgaccgtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 69 - DNA, 20 bp, synthetic construct
  source 1..20: mol_type = other DNA
aggtcagatgatagcatagg

SEQ ID NO: 70 - RNA, 254 nt, synthetic construct
  source 1..254: mol_type = other RNA
aggtcagatgatagcatagggtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 71 - DNA, 20 bp, synthetic construct
  source 1..20: mol_type = other DNA
gaggcctggagtcatggccc

SEQ ID NO: 72 - RNA, 254 nt, synthetic construct
  source 1..254: mol_type = other RNA
gaggcctggagtcatggcccgtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 73 - DNA, 20 bp, synthetic construct
  source 1..20: mol_type = other DNA
ttccaggtgtggacaatgtc

SEQ ID NO: 74 - RNA, 254 nt, synthetic construct
  source 1..254: mol_type = other RNA
ttccaggtgtggacaatgtcgtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 75 - DNA, 20 bp, synthetic construct
  source 1..20: mol_type = other DNA
gactcttcaaatcctccctt

SEQ ID NO: 76 - RNA, 254 nt, synthetic construct
  source 1..254: mol_type = other RNA
gactcttcaaatcctcccttgtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 77 - RNA, 234 nt, synthetic construct
  source 1..234: mol_type = other RNA
gtttcaatcaacctgatctctctgcttgcgctgcaggcttcataagatcagtaggttgaaaagagcatccgtccgacatgcactccgggatggggcagtcccggctcttgcggttcccgatgcagttatgtgtccgcggaacacggtgtcggacacgtcgcaagacaggatgtgagcccattatccacattaggatcaatgtacgcatgagcagtccgcaatgccggggacgct

SEQ ID NO: 78 - DNA, 2977 bp, synthetic construct
  source 1..2977: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagcacatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaagv

SEQ ID NO: 79 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgcgcacatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 80 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENAHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 81 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgaggcgatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 82 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEAILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 83 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagctgatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 84 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENELILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 85 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagcacatcctgccgcgtagtcggtttccgtatgatagctactttaatattctgccggcgtgtgagaagtgcgcgcggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 86 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCARRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 87 - DNA, 2976 bp, synthetic construct
  source 1..2976: mol_type = other DNA
atggatagggagtatctattagaaaaaatacgcgactgtaatccggatgaaatcttcactagcttccaaattgcgaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagcacatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 88 - protein, 992 aa, synthetic construct
MDREYLLEKIRDCNPDEIFTSFQIATGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 89 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgcgagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagcacatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgactctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 90 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVASASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIDSALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 91 - DNA, 2976 bp, synthetic construct
  source 1..2976: mol_type = other DNA
atggatagggagtatctattagaaaaaatacgcgactgtaatccggatgaaatcttcactagcttccaaattgacaccggcgcgaagtactatggttgcaccgtgttggttaaactaccgggggtacagaaaccgattatggtcatcgcggatgtgctgacggctattccaaagatcgaagacattgttgccgctcgttcgctgaatcgcggtacacgtcgcaccaacaagtcgaagaagcgtcgcaaccgcaagctgcgtgcggcgattggtcatatcgacggcgcagataaagttttggcgtattgtaaacgtcgtggcgtgtcctatgctttcgaccaagacgaagaagaaacctcagctgaagataagcgcacccgtaagtcgcgtgaggaggttctggacaaattgaaggtggttgttcgaaatgagatcccggatgagcagcaggacttcgtgtacaagacgtgcgagaaactgctgaacgcggaagagcggaaatgccgtatcaaaaaccgtaacccgtcgaaatgcgactgggaaggttgcaatcaaaacgtgccggcatcgcgaaacgcgatcttcgaaaccctggaacagaccttatatccgtggctgacgcagatttttcgcagctctaagtcctacgtacgtctggaaaacgagcttaacgagtgcgttaaatacttaaacagcctggattatcgtaataaatacagtatagagcagacggacgatgaggagaagcagctggataaacaattaaacaatgaactgaacaaaatccgcgacatcgtgaaatctcacgcatccaaagaacactaccgcgaattcagtcactactggaccaagtattaccgtaaacaactagctaatattttgaaatctagcggcaccggtcgtactacgttttgtcgtcaacattcctccatgtatgtagcgaaacttattgccggagaagtgataccagtgaaacccgaaatctatctgaaagatatcgagggtgcagcgcagaagttgacctatgcaaagttggagagttacctgctgcatagcgttctgccaaccctgaaaacgattggcatcaccaaaattgattccgtgtgtgttgaaagcgcgagcggtagctctccgagcaaagccaaaaaacagaagcgttcagacaaggcgaagaagcaaaaagtgagcaataaaaccaaaaaacagaagtgcagcgatgaggagttccaacaaggcccgatgtttaagttcgacagccgcaaagacatgctgctggcggaattcgataagctgtgtgcatattgcggtgaaccgctcaccggtagctacattgagaacgagcacatcctgccgcgtagtcggtttccgtatgatagctactttaatattctgccggcgtgtgagaagtgcaaccggcgaaagggcgcccgtaccccttatgaagcgggtatgaccattagcacgaaagcgctggatgcattcgatcgttatatcaagaagaaaaagttcgtgcacccgtccatctgggttaaacgtaacctgctgagcggtatgacccatcagacgaacggcgacgccgaacgcgagctgcgttttctgtgccgcaagctgttcatctccggcaaacacacccaaaccccgaaacgcctggctagactgatcgccaccagcctgagcaatgctaccggcatccgtccgcagatcaagtctatcccgcctaagtttgtgtatttttaccgcaacgccgtgctgcccgactttaacaaatacgaagacaaaaagaacggtggtgtggtgaaccacattattgcgtctgcgctgttaggctgcgattttccgtcgccgcactatctgttgtctggtcgttactccggttgtgagaagtaccgtgaattcgttgaccgcttgaaggattcctttgtcgcggtggacgacagcggtatgccgatttgcattccgattagaagcgaaacccaattttttcaagaacaaatcgacaagcgtttgccgggttactctaaaattgacatgagcaagatgaactggaatagcaaacgcaagatcgttagccactaccaaccgcagcgtaaagttggttataaactggtcgcggcgcgtgactctatcgagtctatcaaggaccgtttgctggacctaaacaagaaacaaggcacggatacggttattcgtgacgcgaccatcgagagcattcgtgatgaaaagctcaagtctcggatgctgaacaacccggaccgggctgcttactacttgattgagtatgtgcagcaaagcatcaagtacgggtccaacgaagaggtttaccatccgaccgcaattgagatgaaacagatgcgtgacgagttcgtaaatgatgatccagaactgtacgtcaccgaggacaaggagttgagaaagcaggttccgaacatttttggcatgcgtatccagactggccagagcgaaagccagatctctttccaccgtgtgtgcacccagggtgataccgttcatcaccaccattacatgcaggacatcggcattcgtcatattgcgattgttgttcacattgacaaaaaaggtaaaaagcagaaccaggcaatcactgtgacccgtcaaggtaaggtctacatcggcctgggtacggcgcgtaagagcgttgatcgctcctttggtatgaccctccagggccgtgctttcggtgaaaagggttcccgcaaaaagtgggatcgtcaatggcatcgggagttccgtgcgttctgcaaggctacctggaatgtcgagcgtattcatatcctgaccgcaggctgcgtgttcatcgacactgatggcaccacctacttcgttcgcaatatggatagccaggacaaaggcatccgcattgcgactgcgaaacgtaaattcgtcgaaatcgagcgtgtttttaaaagcccgcacgcctatcagaaatacctcagcaagatgaatgccatcaccaag

SEQ ID NO: 92 - protein, 992 aa, synthetic construct
MDREYLLEKIRDCNPDEIFTSFQIDTGAKYYGCTVLVKLPGVQKPIMVIADVLTAIPKIEDIVAARSLNRGTRRTNKSKKRRNRKLRAAIGHIDGADKVLAYCKRRGVSYAFDQDEEETSAEDKRTRKSREEVLDKLKVVVRNEIPDEQQDFVYKTCEKLLNAEERKCRIKNRNPSKCDWEGCNQNVPASRNAIFETLEQTLYPWLTQIFRSSKSYVRLENELNECVKYLNSLDYRNKYSIEQTDDEEKQLDKQLNNELNKIRDIVKSHASKEHYREFSHYWTKYYRKQLANILKSSGTGRTTFCRQHSSMYVAKLIAGEVIPVKPEIYLKDIEGAAQKLTYAKLESYLLHSVLPTLKTIGITKIDSVCVESASGSSPSKAKKQKRSDKAKKQKVSNKTKKQKCSDEEFQQGPMFKFDSRKDMLLAEFDKLCAYCGEPLTGSYIENEHILPRSRFPYDSYFNILPACEKCNRRKGARTPYEAGMTISTKALDAFDRYIKKKKFVHPSIWVKRNLLSGMTHQTNGDAERELRFLCRKLFISGKHTQTPKRLARLIATSLSNATGIRPQIKSIPPKFVYFYRNAVLPDFNKYEDKKNGGVVNHIIASALLGCDFPSPHYLLSGRYSGCEKYREFVDRLKDSFVAVDDSGMPICIPIRSETQFFQEQIDKRLPGYSKIDMSKMNWNSKRKIVSHYQPQRKVGYKLVAARDSIESIKDRLLDLNKKQGTDTVIRDATIESIRDEKLKSRMLNNPDRAAYYLIEYVQQSIKYGSNEEVYHPTAIEMKQMRDEFVNDDPELYVTEDKELRKQVPNIFGMRIQTGQSESQISFHRVCTQGDTVHHHHYMQDIGIRHIAIVVHIDKKGKKQNQAITVTRQGKVYIGLGTARKSVDRSFGMTLQGRAFGEKGSRKKWDRQWHREFRAFCKATWNVERIHILTAGCVFIDTDGTTYFVRNMDSQDKGIRIATAKRKFVEIERVFKSPHAYQKYLSKMNAITK

SEQ ID NO: 93 - RNA, 20 nt, synthetic construct
  source 1..20: mol_type = other RNA
atgactccaggcctccccaa

SEQ ID NO: 94 - RNA, 258 nt, synthetic construct
  source 1..258: mol_type = other RNA
gatgactccaggcctccccaagttgtattcggatgggagtaaaaggttgagtacacctggaaacaaaacgacataaaggtgttctcccatctgtgtacaaagagcagatatatgagttgataagtaatatgttgcacgtatggactttgacagtctgtacctctgcgtgaaagacatattgcgttattcatatatctgtgagtctgttctaaatttaaaggtttagggaaggctcaccttcgggtgggcctttctgcg

SEQ ID NO: 95 - DNA, 10 bp, synthetic construct
  source 1..10: mol_type = other DNA
ctgtggggcc

SEQ ID NO: 96 - DNA, 10 bp, synthetic construct
  source 1..10: mol_type = other DNA
agcctggcca

SEQ ID NO: 97 - DNA, 100 bp, synthetic construct; identical to SEQ ID NO: 43
  source 1..100: mol_type = other DNA

SEQ ID NO: 98 - DNA, 200 bp, synthetic construct; identical to SEQ ID NO: 44
  source 1..200: mol_type = other DNA

SEQ ID NO: 99 - DNA, 340 bp, synthetic construct
  source 1..340: mol_type = other DNA
gctcaaagctttagactggaggtaccgtagtactatgagcttaatcgcgctgagggtgatttgggtgttcgtctcatatcttcgattatcggacagtagtctgtggggccatgactccaggcctccccaaagcctggccaagtagtatcggaccggttggcgaaccacagcgtataacgagacatggactcgcattccctaccttaagcgggcggttttggagctcggaattcatgtaaactcgcccgtttaacataatctagatggatcttgcgcgcaccgcccgaacaccactcgccacaaaaaaccgccggaacgtccaaaagtacgggttttgctg

SEQ ID NO: 100 - DNA, 27 bp, synthetic construct
  source 1..27: mol_type = other DNA
gctcaaagctttagactggaggtaccg

SEQ ID NO: 101 - DNA, 24 bp, synthetic construct
  source 1..24: mol_type = other DNA
cagcaaaacccgtacttttggacg